(19) United States
(12) Patent Application Publication
(10) Pub. No.: US 2003/0228603 A1
(43) Pub. Date: Dec. 11, 2003

(54) Compositions selective for caffeine or aspartame and methods of using same

(76) Inventors: Sharon T. Cload, Cambridge, MA (US); Alicia Ferguson, Somerville, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/406,903

(22) Filed: Apr. 3, 2003

Related U.S. Application Data
(60) Provisional application No. 60/370,266, filed on Apr. 5, 2002.
(60) Provisional application No. 60/398,858, filed on Jul. 25, 2002.

(51) Int. Cl. C12Q 1/68; C07H 21/04

(52) U.S. Cl. 435/6; 536/23.1

(57) ABSTRACT

Compositions which recognize and report on the concentration of caffeine or aspartame target molecules. The invention further relates to methods of using the compositions to monitor the presence or concentration of such targets in a variety of samples, including those samples to be ingested, such as beverages, e.g., coffee or soft drinks.

### RELATED APPLICATIONS

This application claims priority to provisional patent application U.S. S. No. 60/370,266 filed on Apr. 5, 2002, which is incorporated herein by reference in its entirety.

### FIELD OF THE INVENTION

The invention relates to compositions which selectively recognize caffeine or artificial sweeteners as target molecules. The invention further relates to methods of using the compositions to monitor the presence or concentration of such targets in a variety of samples. Samples include those to be ingested or consumed, such as beverages, e.g., coffee or soft drinks.

### BACKGROUND OF THE INVENTION

Detection and measurement of components in a solution is an important aspect of production and quality control of substances to be used in foodstuffs and beverages.

There is a need in the art for expedient, accurate, efficient, cost-effective methods of monitoring the presence or concentration of analytes in a variety of samples, including those to be ingested or consumed.

### SUMMARY OF THE INVENTION

The nucleic acid compositions of the present invention are used to monitor the presence or concentration of analytes. The invention also provides for accurate, efficient, cost-effective methods for detecting the presence or concentration of analytes in compositions using the methods of the invention.

The present invention includes nucleic acid compositions, referred to as nucleic acid sensor molecules (“NASMs”), which have a target modulation domain, a linker domain and a catalytic domain. In one embodiment of the invention, the target modulation domain of the NASM recognizes caffeine. In another embodiment of the invention, the target modulation domain of the NASM recognizes aspartame. The NASMs of the present invention can be made from RNA, DNA, or a combination of RNA and DNA. NASMs according to the present invention can also include at least one modified nucleotide.

The catalytic domain of the NASMs according to the present invention can include a unit that generates an optical signal. In some embodiments, this unit can include a first optical signaling moiety, such as fluorescent donor, and a second signaling moiety, such as a fluorescent quencher. In embodiments having first and second optical signaling moieties, recognition of a target by the target modulation domain can change the proximity between the optical moieties. For example, in embodiments having a fluorescent donor and a fluorescent quencher, recognition of a target by the target modulation domain can result in an increase in the detectable fluorescence of the fluorescent donor. In other embodiments, recognition of a target by the target modulation domain can result in a conformational change in the optical signaling moiety, thereby resulting in a detectable optical signal.

In another embodiment, the catalytic domain of the NASMs of the present invention can include a ribozyme. For example, the catalytic domain can include an endonucleolytic ribozyme, such as a cis-endonucleolytic ribozyme or a trans-endonucleolytic ribozyme. In a preferred embodiment, the endonucleolytic ribozyme of the catalytic domain is a hammerhead ribozyme. In other embodiments, the catalytic domain of the NASMs can include a self-ligating ribozyme, such as for example, a cis-ligase ribozyme, a trans-ligase, a 1-piece ligase, a 2-piece ligase, a 3-piece ligase or any combination thereof.

NASMs of the present invention can also include an additional label. In one embodiment, the NASM can include a detectable label. For example, the detectable label can include at least one radioactive moiety, or a fluorescent label, such as for example, fluorescein, DABCYL, or a green fluorescent protein (GFP) moiety. In another embodiment, the NASM of the present invention can include an affinity capture tag label.

NASMs according to the present invention can be used to form compositions. In some embodiments, these compositions can also include an RNase inhibitor, such as for example, Va-riboside, vanadyl, tRNA, polyu, RNaseln or RNaseOut. In these embodiments, the compositions can be substantially RNase-free.

In another embodiment, at least one NASM in the compositions according to the present invention can be affixed to a substrate, such as for example, glass, gold or other metal(s), silicon or other semiconductor material(s), nylon or plastic. These compositions can be attached to the substrate either covalently or non-covalently. In one embodiment, one or more NASM according to the present invention can be immobilized to the substrate by hybridization of an end portion of the NASM to an oligonucleotide that is attached to the surface of the substrate. In this embodiment, virtually any number of NASMs can be immobilized via hybridization, but in a preferred embodiment, at least 50 NASMs are attached to the substrate, and more preferably, at least 250 NASMs are attached to the substrate.

The present invention also provides systems, diagnostic systems and methods for identifying or detecting a target molecule in a samples composition having a target modulation domain which recognizes the target molecule by using a NASM composition according to the present invention and a detector that is in communication with the composition. In this embodiment, the detector is capable of detecting a signal that is generated by the composition when the NASM recognizes a target molecule. In one embodiment, the systems, diagnostic systems and methods are used to detect caffeine, and in another embodiment, the systems, diagnostic systems and methods are used to detect aspartame. In some embodiments, the systems and methods can include a light source that is in optical communication with the composition containing the target molecule. In some embodiments, the systems and methods can also include a processor for processing the optical signals detected by the detector. The change in signal generated by the NASM composition can be used to quantify the amount of the target molecule, such as for example caffeine or aspartame, in a sample. Suitable samples for use in conjunction with these systems and methods for detecting target molecules such as caffeine and aspartame include, but are not limited to, a process solution or a beverage, such as for example, coffee or a soft drink.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1A is a schematic representation of secondary structure representation of 3-piece NASM construct. FIG. 1B is a schematic representation of a 1-piece NASM construct which is a slightly modified version of 3-piece system where the effector and substrate regions are replaced by a stable GNRA tetraloop.

FIG. 2 is a schematic representation of a secondary structure representation of two 2-piece NASMs with their oligonucleotide substrate.

FIG. 3 is a flow diagram showing a gel-based method for selecting nucleic acid sensor molecules having a target molecule activatable endonuclease activity.

FIG. 4 is a flow diagram showing a method for selecting nucleic acid sensor molecules having a target molecule activatable ligase activity.

FIG. 5 is a flow diagram showing a method for selecting nucleic acid sensor molecules having a target molecule activatable self-cleavage activity.

FIG. 6 is a schematic representation of various FRET formats in hammerhead ribozymes.

FIG. 7A is a schematic representation of an example of a self-cleaving nucleic acid sensor molecule bound to a solid support when used in an epi-illuminated FRET detection scheme. FIG. 7B is a schematic representation of the same sensor in an epi-illuminated beacon configuration, with the acceptor fluorophore replaced by a quencher group. FIG. 7C is a schematic representation of the same sensor in an TIR-illuminated beacon configuration.

FIG. 8 is a schematic representation of the conversion of a core hammerhead NASM into optical NASMs useful for FRET.

FIG. 9 is a schematic representation of stem I-modified NASMs useful for FRET.

FIG. 10 is a schematic representation of immobilized hammerhead NASMs useful for FRET.

FIG. 11A is a graph depicting fluorescence intensity vs. time for cleavage in optical hammerhead NASM as measured by FRET. FIG. 11B is a line graph of first order kinetic analysis of cleavage rate as measured by FRET.

FIG. 12 is a schematic representation of the use of beads in a homogeneous assay format utilizing a self-ligating nucleic acid sensor. FIG. 12A is a schematic representation of the beads prior to target binding and ligation (no emission from acceptor). FIG. 12B is a schematic representation of the beads after target binding and ligation (emission from acceptor detected).

FIG. 13A is a schematic representation of an example of a self-ligating nucleic acid sensor molecule bound to a solid support when used in a TIR-illuminated detection scheme where there is a signal increase upon target binding. FIG. 13B is a schematic representation of the same sensor in an epi-illuminated configuration, where target binding is detected by monitoring changes of the fluorophore bound to the substrate at the surface of the array. FIG. 13C is a schematic representation of the same epi-illuminated configuration, where target binding is detected by monitoring changes in the fluorescence polarization.

FIG. 14 is a schematic representation of a NASM of a ligase ribozyme tethered to a chip by a capture oligonucleotide.

FIG. 15 is a schematic representation of a solid phase self-ligating NASM-ECD chip used for electrochemical detection.

FIG. 16 is a schematic representation of a solid-phase self-cleaving NASM-ECD ship used for electrochemical detection.

FIG. 17 is a schematic representation of a peak in the faradaic current, centered at the redox potential of the electron donor species (specified for a given reference electrode) and superimposed on top of the capacitive current baseline which is observed in the absence of surface-immobilized signaling probes.

FIG. 18A is a schematic representation of the HH₃₃WT pool and FIG. 18B is a schematic representation of the HH₃₃AG pool.

FIG. 19 is a bar graph depicting the early rounds of strategy 2 selections for caffeine dependent NASMs. The % cleavage in both the negative (− target) and positive (+ target) steps of each round of selection are plotted.

FIG. 20 is a bar graph depicting the progress of the activity-based caffeine selection. In each round, beginning with Round 8, the activity of the pool in the presence and in the absence of 5 mM caffeine was measured. Ratios >1 indicate increased activity in the presence of 5 mM caffeine.

FIG. 21 is a bar graph depicting the early rounds of strategy 2 selections for aspartame dependent NASMs. The % cleavage in both the negative (− target) and positive (+ target) steps of each round of selection are plotted. After 16 rounds, the aspartame selection showed minimal target-dependent activity.

FIG. 22 is a bar graph depicting the elution of RNA in the presence of 5 mM caffeine.

FIG. 23 is a bar graph depicting the elution of RNA in the presence of 5 mM aspartame.

FIG. 24 is a schematic representation of the origin of the 8 pools carried forward into activity-based selection.

FIG. 25 is a bar graph depicting the activity-based phase of the strategy 3 selections for caffeine.

FIG. 26 is a bar graph depicting the activity-based phase of the strategy 3 selections for aspartame.

FIG. 27 is a bar graph depicting the cleavage activity of various caffeine sensors.

FIG. 28 is a bar graph depicting the cleavage activity of various aspartame sensors.

FIG. 29 depicts the structure of the cGMP-dependent hammerhead construct used for the FRET assay.

FIG. 30 is a line graph depicting the FRET signal with the Alexa 594/Cy5 dye (combination A) upon incubation, with and without cGMP target. The signal was recorded real-time on a Packard Fusion plate reader, using a 570 nm (20 nm bandpass) exitation and a 620 nm (10) emission filter set.

FIG. 31A is a line graph depicting the analysis of a FRET real-time measurement with curve-fitting to a 1^{st} order kinetic model, allowing calculation of the rate constant for cleavage; FIG. 31B is a line graph depicting the rates and cGMP concentrations on a double logarithmic scale, showing a linear correlation.

FIG. 32A is a generic representation of the hammerhead ribozyme constructs used for selection and FIG. 32B is a generic representation of the hammerhead ribozyme structures used for FRET experiments.

FIG. 33A is a line graph indicating the caffeine-dependent FRET of clone S2.caf.D11 in assay buffer pH 7.0, 10 mM MgCl₂; FIG. 33B is a line graph indicating the log of the rate of cleavage in the presence of caffeine.

FIG. 34A is a line graph indicating the caffeine-dependent FRET of clone S2.caf.D11 in assay buffer pH 7.0, 25 mM MgCl₂; FIG. 34B is a line graph indicating the log of the rate of cleavage in the presence of caffeine.

FIG. 35A is a line graph indicating the caffeine-dependent FRET of clone S2.caf.D11 in assay buffer pH 7.0, 25 mM MgCl₂, with a read time of 5 minutes; FIG. 35B is a line graph indicating the log of the rate of cleavage in the presence of caffeine.

FIG. 36 is a line graph showing the pH-dependence of the caffeine sensor S2.caf.D11 under standard conditions in assay buffer (10 mM MgCl₂) at variable pH.

FIG. 37 is a graph of aspartame-dependent FRET experiment using S3.asp.A2 in selection buffer pH 7.0, 10 mM MgCl₂. The analyses based on read-times of 5 min (37A), 10 min (37B), and 70 min (37° C.) show considerable variations in rates, as indicated in 37D.

FIG. 38 is a line graph of a FRET assay using S3.asp.A2 in assay buffer pH 7.5, 25 mM MgCl₂ containing either 1 mM aspartame, phenylalanine, or no target.

FIG. 39A is a line graph indicating the aspartame-dependent FRET experiment of clone S3.asp.E4 in assay buffer pH 7.5, 40 mM MgCl₂ with a read time of 30 minutes; FIG. 39B is a line graph indicating the log of the rate of cleavage in the presence of aspartame.

FIG. 40A is a line graph indicating the aspartame-dependent FRET experiment of clone S3.asp.E4 in assay buffer pH 7.5, 40 mM MgCl₂ with a read time of 5:45 min.; FIG. 40B is a line graph indicating the log of the rate of cleavage in the presence of aspartame.

FIG. 41 is a line graph indicating the dose-response of FRET assay using S3.asp.E4 in assay buffer pH 7.5, 25 mM MgCl₂ the rates were derived with data generated over 20 minutes.

FIG. 42 is a line graph of a FRET assay using S3.asp.E4 in assay buffer pH 7.5, 25 mM MgCl₂ containing either 1 mM aspartame, phenylalanine, or no target.

FIG. 43 shows a graph of the cGMP SPReeta Assay, FIG. 43A shows the immobilization of the cGMP-dependent NASM to a neutravidin coated surface, and FIG. 43B shows initiation of cleavage by the addition of cGMP.

FIG. 44 is a schematic representation of the immobilized, modified cGMP NASM reaction with cGMP.

FIG. 45 is a line graph of the immobilized, modified cGMP NASM reaction with 0, 100 μM, and 600 μM cGMP over time.

FIG. 46A is a line graph of the rate of cleavage of the immobilized, modified cGMP NASM reactions at various concentrations of cGMP; FIG. 46B is a series of line graphs depicting raw rate data.

### DETAILED DESCRIPTION OF THE INVENTION

The invention is drawn to aptamer nucleic acid molecules (“aptamers”) which selectively recognize target molecules such as caffeine or artificial sweeteners, such as aspartame. The invention also relates to catalytic nucleic acid sensor molecules (also known as allosteric ribozymes, aptazymes, and the like) and to optical nucleic acid sensor molecules which selectively recognize these targets.

Catalytic nucleic acid sensor molecules (NASMs) can be generated in a number of ways, including use of an aptamer derived target modulation domain joined to a catalytic domain by a linker region. Optical NASMs are generated from catalytic NASMs by addition of an optical signal generating unit. In general, optical NASMs generate a detectable optical signal upon recognition of a target molecule.

The invention also includes methods by which a change in the conformation of a nucleic acid composition of the invention upon recognition of a specific target molecule can be coupled to a quantifiable, measurable signal.

The invention also includes methods which allow one to assay the presence or concentration of a target in a sample. Assays can be carried out in a variety of formats, including assays on chips or other substrates or in solution. These assays have applications in detection or quantitation of the target in a sample, such as a bodily fluid, or a food or beverage product to be consumed by a subject. The methods described herein also have application in quality control of sample production, including the production of samples to be ingested by a subject.

High throughput screening methods are also provided. A plurality of nucleic acid molecules of the invention are immobilized at discrete sites on a substrate, e.g., on a 96- or 384-well plate. Such a plurality of immobilized nucleic acid compositions can be used to detect many different targets simultaneously, or can be used to monitor different solution or reaction conditions (e.g., different buffers, or the presence of different target concentrations).

One aspect of the invention concerns the detection of components of beverages, such as soft drinks. For example, the compositions and methods of the invention can be used to detect the presence or concentration of caffeine in beverages, e.g., coffee or cola soft drinks. The compositions and methods of the invention can also be used to detect the presence or concentration of aspartame in sugar-free beverages, such as diet soft drinks. Preferably, the compositions of the invention are used to detect the target molecules in a concentration range of 0.5 μM to 5 mM in a beverage sample. In preferred aspects of the invention, the presence or concentration of the target (e.g., caffeine or aspartame) is measured in 5 minutes or less, preferably in 3 minutes or less. The presence or concentration of the target (e.g., caffeine or aspartame) is preferably measured without a dilution step to adjust the pH or salt concentration of the sample.

Nucleic acid compositions of the invention (aptamers and nucleic acid sensor molecules) are RNAs, DNAs, RNA/DNA hybrids, or derivatives or analogs of nucleic acids that catalyze a chemical reaction and/or undergo a conformational change upon the recognition of a specific target molecule.

Nucleic acid compositions of the invention can be generated or selected by a variety of methods both disclosed herein and known in the art. For examples, see WO98/27104, WOO 1/96559, and WO 00/26226, each of which is incorporated herein by reference. Three separate strategies were employed to generate NASM detection systems of the present invention. In brief, strategy one involved first identifying aptamers (based on target binding affinity) to the target molecules using a standard pool (N₄₀APT), followed by using those aptamer sequences to design NASM molecules. In strategy two, pool molecules comprised of a hammerhead ribozyme core appended with a randomized target binding domain (pool designations HH₃₃WT and HH₃₃AG) were subjected to selection on the basis of target-dependent cleavage activity. In strategy 3 (two phase selection), hammerhead based pools were first enriched for binding to target, then subjected to selection on the basis of target-dependent activity.

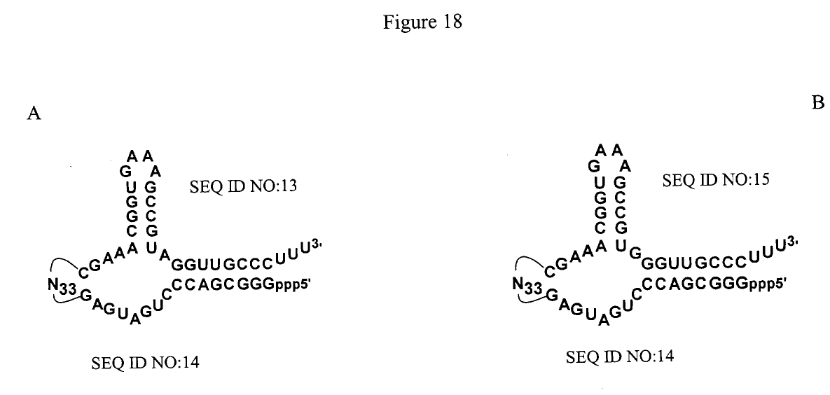

Synthesis of three different RNA pools, N₄₀APT, HH₃₃WT, and HH₃₃AG (Eckstein et al., RNA Structure and Function (1998) Cold Spring Harbor Laboratory Press, pg. 341) was performed, as described below. The N₄₀APT pool contained sequences with a 5′ oligonucleotide linked to a randomized region of 40 nucleotides which is linked to a 3′ oligonucleotide. The HH₃₃WT, and HH₃₃AG. pools contained sequences with a 5′ oligonucleotide linked to a randomized region of 33 nucleotides which is linked to a 3′ oligonucleotide, and are shown schematically in FIG. 18. Transcription conditions for each pool were optimized and sufficient quantities of RNA to carry all of the selections were prepared. Each selection was initiated with approximately 4×10¹⁵ RNA molecules (6.6 nmoles).

Definitions

In order to more clearly and concisely describe and point out the subject matter of the claimed invention, the following definitions are provided for specific terms which are used in the following written description and the appended claims.

As defined herein, “nucleic acid” means either DNA, RNA, single-stranded or double-stranded, and any chemical modifications thereof. Modifications include, but are not limited to, those which provide other chemical groups that incorporate additional charge, polarizability, hydrogen bonding, electrostatic interaction, and fluxionality to the nucleic acid ligand bases or to the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping.

As defined herein, a “oligonucleotide” is used interchangeably with the term “nucleic acid” and includes RNA or DNA (or RNA/DNA) sequences of more than one nucleotide in either single strand or double-stranded form. A “modified oligonucleotide”. includes at least one nucleotide residue with any of: an altered internucleotide linkage(s), altered sugar(s), altered base(s), or combinations thereof.

As defined herein, “target” means any compound or molecule of interest for which a diagnostic test is desired and where a nucleic acid ligand is known or can be identified. A “target” is any molecule to be detected, and is any molecule for which a nucleic acid ligand exists or can be generated. A target molecule can be naturally occurring or artificially created, including a protein, peptide, carbohydrate, polysaccharide, glycoprotein, hormone, receptor, antigen, antibody, virus, substrate, metabolite, transition state analog, cofactor, inhibitor, drug, dye, nutrient, growth factor, etc. without limitation.

As defined herein, a molecule which “naturally binds to DNA or RNA”. is one which is found within a cell in an organism found in nature.

As defined herein, a “random sequence” or a “randomized sequence” is a segment of a nucleic acid having one or more regions of fully or partially random sequences. A fully random sequence is a sequence in which there is an approximately equal probability of each base (A, T, C, and G) being present at each position in the sequence. In a partially random sequence, instead of a 25% chance that an A, T, C, or G base is present at each position, there are unequal probabilities.

As defined herein, a “fixed region” is a nucleic acid sequence which is known.

As defined herein, a “signal”. is a detectable physical quantity, impulse or object.

As defined herein, an “optical signal” is a signal the optical properties of which can be detected.

As defined herein, “test mixture” refers to any sample that contains a plurality of molecules. This includes, but is not limited to, samples from process solutions used in the production of various food stuffs and beverages, bodily fluids, and any sample for environmental and toxicology testing such as contaminated water and industrial effluent.

As defined herein, “fluorescent group” refers to a molecule that, when excited with light having a selected wavelength, emits light of a different wavelength. Fluorescent groups include, but are not limited to, fluorescein, tetramethylrhodamine, Texas Red, BODIPY, 5-[(2-aminoethyl)amino]napthalene-1-sulfonic acid (EDANS), and Lucifer yellow. Fluorescent groups may also be referred to as “fluorophores”.

As defined herein, “fluorescence-modifying group” refers to a molecule that can alter in any way the fluorescence emission from a fluorescent group. A fluorescence-modifying group generally accomplishes this through an energy transfer mechanism. Depending on the identity of the fluorescence-modifying group, the fluorescence emission can undergo a number of alterations, including, but not limited to, attenuation, complete quenching, enhancement, a shift in wavelength, a shift in polarity, a change in fluorescence lifetime. One example of a fluorescence-modifying group is a quenching group.

As defined herein, “energy transfer” refers to the process by which the fluorescence emission of a fluorescent group is altered by a fluorescence-modifying group. If the fluorescence-modifying group is a quenching group, then the fluorescence emission from the fluorescent group is attenuated (quenched). Energy transfer can occur through fluorescence resonance energy transfer, or through direct energy transfer. The exact energy transfer mechanisms in these two cases are different. It is to be understood that any reference to energy transfer in the instant application encompasses all of these mechanistically-distinct phenomena.

As defined herein, “energy transfer pair” refers to any two molecules that participate in energy transfer. Typically, one of the molecules acts as a fluorescent group, and the other acts as a fluorescence-modifying group. The preferred energy transfer pair of the instant invention comprises a fluorescent group and a quenching group. In some cases, the distinction between the fluorescent group and the fluorescence-modifying group may be blurred. For example, under certain circumstances, two adjacent fluorescein groups can quench one another's fluorescence emission via direct energy transfer. For this reason, there is no limitation on the identity of the individual members of the energy transfer pair in this application. All that is required is that the spectroscopic properties of the energy transfer pair as a whole change in some measurable way if the distance between the individual members is altered by some critical amount.

“Energy transfer pair” is used to refer to a group of molecules that form a single complex within which energy transfer occurs. Such complexes may comprise, for example, two fluorescent groups which may be different from one another and one quenching group, two quenching groups and one fluorescent group, or multiple fluorescent groups and multiple quenching groups. In cases where there are multiple fluorescent groups and/or multiple quenching groups, the individual groups may be different from one another e.g., one complex contemplated herein comprises fluorescein and EDANS as fluorescent groups, and DABCYL as a quenching agent.

As defined herein, “quenching group” refers to any fluorescence-modifying group that can attenuate at least partly the light emitted by a fluorescent group. We refer herein to this attenuation as “quenching”. Hence, illumination of the fluorescent group in the presence of the quenching group leads to an emission signal that is less intense than expected, or even completely absent. Quenching occurs through energy transfer between the fluorescent group and the quenching group. The preferred quenching group of the invention is (4-dimethylamino-phenylazo)benzoic acid (DABCYL).

As defined herein, “fluorescence resonance energy transfer” or “FRET” refers to an energy transfer phenomenon in which the light emitted by the excited fluorescent group is absorbed at least partially by a fluorescence-modifying group. If the fluorescence-modifying group is a quenching group, then that group can either radiate the absorbed light as light of a different wavelength, or it can dissipate it as heat. FRET depends on an overlap between the emission spectrum of the fluorescent group and the absorption spectrum of the quenching group. FRET also depends on the distance between the quenching group and the fluorescent group. Above a certain critical distance, the quenching group is unable to absorb the light emitted by the fluorescent group, or can do so only poorly.

As defined herein, “direct energy transfer” refers to an energy transfer mechanism in which passage of a photon between the fluorescent group and the fluorescence-modifying group does not occur. Without being bound by a single mechanism, it is believed that in direct energy transfer, the fluorescent group and the fluorescence-modifying group interfere with each others electronic structure. If the fluorescence-modifying group is a quenching group, this will result in the quenching group preventing the fluorescent group from even emitting light.

In general, quenching by direct energy transfer is more efficient than quenching by FRET. Indeed, some quenching groups that do not quench particular fluorescent groups by FRET (because they do not have the necessary spectral overlap with the fluorescent group) can do so efficiently by direct energy transfer. Furthermore, some fluorescent groups can act as quenching groups themselves if they are close enough to other fluorescent groups to cause direct energy transfer. For example, under these conditions, two adjacent fluorescein groups can quench one another's fluorescence effectively. For these reasons, there is no limitation on the nature of the fluorescent groups and quenching groups useful for the practice of this invention.

As defined herein, an “aptamer” is a nucleic acid which binds to a non-nucleic acid target molecule or a nucleic acid target through non-Watson-Crick base pairing.

As defined herein, an aptamer nucleic acid molecule which “recognizes a target molecule” is a nucleic acid molecule which specifically binds to a target molecule.

As defined herein, a “nucleic acid sensor molecule” or “NASM” refers to either or both of a catalytic nucleic acid sensor molecule and an optical nucleic acid sensor molecule.

As defined herein, a “catalytic nucleic acid sensor molecule” is a nucleic acid sensor molecule comprising a target modulation domain, a linker region, and a catalytic domain.

As defined herein, an “optical nucleic acid sensor molecule” is a catalytic nucleic acid sensor molecule wherein the catalytic domain has been modified to emit an optical signal as a result of and/or in lieu of catalysis by the inclusion of an optical signal generating unit.

As defined herein, a “nucleic acid ligand” refers to either or both an aptamer or a NASM.

As defined herein, a “target modulation domain” (TMD) is the portion of a nucleic acid sensor molecule which recognizes a target molecule. The target modulation domain is also sometimes referred to herein as the “target activation site” or “effector modulation domain”.

As defined herein, a “catalytic domain” is the portion of a nucleic acid sensor molecule possessing catalytic activity which is modulated in response to binding of a target molecule to the target modulation domain.

As defined herein, a “linker region” or “linker domain” is the portion of a nucleic acid sensor molecule by or at which the “target modulation domain” and “catalytic domain” are joined. Linker regions include, but are not limited to, oligonucleotides of varying length, base pairing phosphodiester, phosphothiolate, and other covalent bonds, chemical moieties (e.g., PEG), PNA, formacetal, bismaleimide, disulfide, and other bifunctional linker reagents. The linker domain is also sometimes referred to herein as a “connector” or “stem”.

As defined herein, an “optical signal generating unit” is a portion of a nucleic acid sensor molecule comprising one or more nucleic acid sequences and/or non-nucleic acid molecular entities, which change optical or electrochemical properties or which change the optical or electrochemical properties of molecules in close proximity to them in response to a change in the conformation or the activity of the nucleic acid sensor molecule following recognition of a target molecule by the target modulation domain.

As defined herein, a nucleic acid sensor molecule which “recognizes a target molecule” is a nucleic acid molecule whose activity is modulated upon binding of a target molecule to the target modulation domain to a greater extent than it is by the binding of any non-target molecule or in the absence of the target molecule. The recognition event between the nucleic acid sensor molecule and the target molecule need not be permanent during the time in which the resulting allosteric modulation occurs. Thus, the recognition event can be transient with respect to the ensuing allosteric modulation (e.g., conformational change) of the nucleic acid sensor molecule.

As defined herein, a “cleavage substrate” is an oligonucleotide or portion of an oligonucleotide cleaved upon target molecule recognition by a target modulation domain of an endonucleolytic nucleic acid sensor molecule.

As defined herein, an “oligonucleotide substrate” is an oligonucleotide that is acted upon by the catalytic domain of a nucleic acid sensor molecule with ligase activity.

As defined herein, an “effector oligonucleotide” is an oligonucleotide that base pairs with the effector oligonucleotide binding domain of a nucleic acid sensor molecule with ligase activity.

As defined herein, an “effector oligonucleotide binding domain” is the portion of the nucleic acid sensor molecule with ligase activity which is complementary to the effector oligonucleotide.

As defined herein, a “capture oligonucleotide” is an oligonucleotide that is used to attach a nucleic acid sensor molecule to a substrate by complementarity and/or hybridization.

As defined herein, an “oligonucleotide substrate binding domain” is the portion on the nucleic acid sensor molecule with ligase activity that is complementary to and can base pair with an oligonucleotide substrate.

As defined herein, a “oligonucleotide supersubstrate”. is an oligonucleotide substrate that is complementary to and can base pair with the oligonucleotide substrate binding domain and to the effector oligonucleotide binding domain of a nucleic acid sensor molecule with ligase activity. The oligonucleotide supersubstrate may or may not carry an affinity tag.

As defined herein, a “oligonucleotide supersubstrate binding domain”. is the region of a nucleic acid sensor molecule with ligase activity that is complementary to and can base pair with the oligonucleotide supersubstrate.

As defined herein, “switch factor” is the enhancement observed in the catalytic activity and/or catalytic initial rate of a nucleic acid sensor molecule upon recognition of a target molecule by the target modulation domain.

As defined herein, an “amplicon” is the sequence of a nucleic acid sensor molecule with ligase activity covalently ligated to an oligonucleotide substrate.

As defined herein, “amplicon dependent nucleic acid amplification” refers to a technique by which one can amplify the signal of a nucleic acid sensor molecule by use of standard RT/PCR or Real-Time RT-PCR methods.”

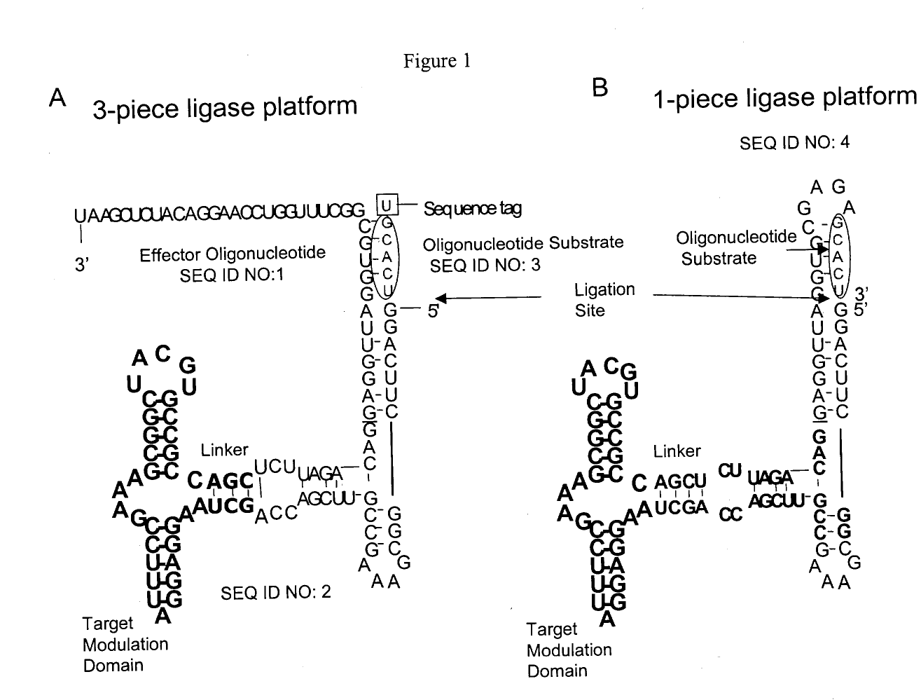

As defined herein, a “3-piece ligase” is a 3-component trans-ligase ribozyme. The first component consists of the catalytic domain, the linker, the target modulation domain, the substrate binding domain and the effector oligonucleotide binding domain. The second component is the effector oligonucleotide that is complementary to the effector oligonucleotide binding domain. The third component is the oligonucleotide substrate that is complementary to the substrate binding domain. This system follows the format of the 3-piece ligase platform shown in FIG. 1A.

As defined herein, a “cis-ligase ribozyme” is a ligase ribozyme that ligates its 3′ end to its 5′ end. The cis-ligase ribozyme is also referred herein as “l-piece ligase” and is a 1-component system where oligonucleotide substrate, oligonucleotide substrate binding domain, catalytic domain, effector oligonucleotide and effector oligonucleotide binding domains are fused in the format shown in FIG. 1B.

As defined herein, a “trans-ligase ribozyme”. is a ligase ribozyme that ligates its 5′ end to the 3′ end of an oligonucleotide substrate.

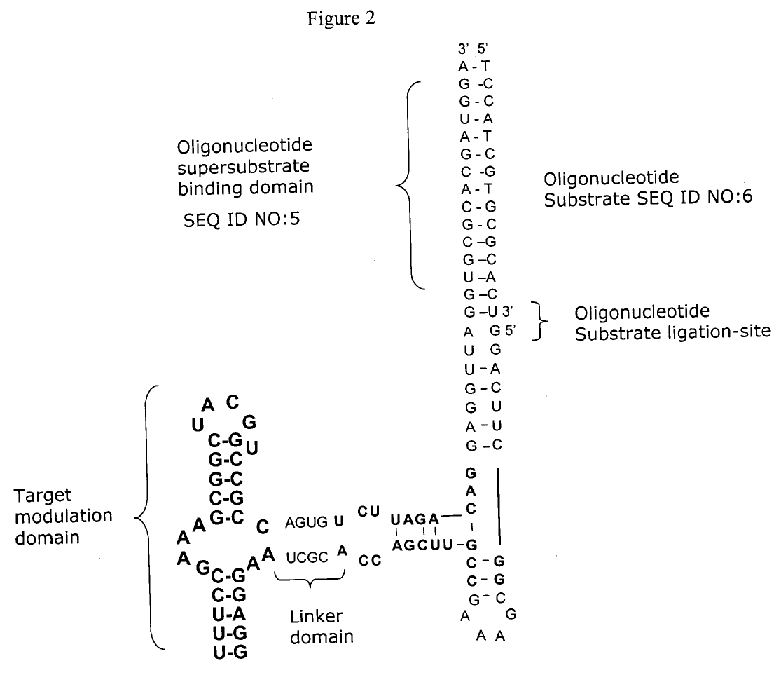

As defined herein, a “2-piece ligase” is a 2-component trans-ligase ribozyme. The first component consists of the catalytic domain, the linker region, the target modulation domain, the substrate binding domain and the effector oligonucleotide binding domain. The second component is the oligonucleotide substrate that is complementary to the substrate binding domain and the effector oligonucleotide binding domain. This system follows the format shown in FIG. 2.

As defined herein, “stem selection” refers to a process performed on a pool of nucleic molecules comprising a target modulation domain, a catalytic domain and an oligonucleotide linker region wherein the linker region is fully or partially randomized.

As defined herein, “rational design/engineering” refers to a technique used to construct nucleic acid sensor molecules in which a non-conserved region of a ribozyme is replaced with a target modulation domain and joined to the catalytic domain of the ribozyme by an oligonucleotide linker region.

As defined herein, a “biosensor” comprises a plurality of nucleic acid ligands.

As defined herein, “substrate” means any physical supporting surface, whether rigid, flexible, solid, porous, gel-based, or of any other material or composition. A substrate includes a microfabricated solid surface to which molecules may be attached through either covalent or non-covalent bonds. This includes, but is not limited to, Langmuir-Bodgett films, functionalized glass, membranes, charged paper, nylon, germanium, silicon, PTFE, polystyrene, gallium arsenide, gold, and silver. Any other material known in the art that is capable of having functional groups such as amino, carboxyl, thiol or hydroxyl incorporated on its surface, is contemplated. This includes surfaces with any topology, such spherical surfaces and grooved surfaces.

As defined herein, an “array” or “microarray” refers to a biosensor comprising a plurality of nucleic acid sensor molecules immobilized on a substrate.

As defined herein, “specificity” refers to the ability of a nucleic acid of the present invention to recognize and discriminate among competing or closely-related targets or ligands. The degree of specificity of a given nucleic acid is not necessarily limited to, or directly correlated with, the binding affinity of a given molecule. For example, hydrophobic interaction between molecule A and molecule B has a high binding affinity, but a low degree of specificity. A nucleic acid that is 100 times more specific for target A relative to target B will preferentially recognize and discriminate for target A 100 times better than it recognizes and discriminates for target B.

As defined herein, “selective” refers to a molecule that has a high degree of specificity for a target molecule.

I. Nucleic Acid Compositions

In addition to carrying genetic information, nucleic acids can adopt complex three-dimensional structures. These three-dimensional structures are capable of specific recognition of target molecules and, furthermore, of catalyzing chemical reactions. Nucleic acids will thus provide candidate detection molecules for diverse target molecules, including those which do not naturally recognize or bind to DNA or RNA.

In aptamer selection, combinatorial libraries of oligonucleotides are screened in vitro to identify oligonucleotides which bind with high affinity to pre-selected targets. In NASM selection, on the other hand, combinational libraries of oligonucleotides are screened in vitro to identify oligonucleotides which exhibit increased catalytic activity in the presence of targets. Possible target molecules for both aptamers and NASMs include natural and synthetic polymers, including proteins, polysaccharides, glycoproteins, hormones, receptors, and cell surfaces, and small molecules such as drugs, metabolites, transition state analogs, specific phosphorylation states, and toxins. Small biomolecules, e.g., amino acids, nucleotides, NAD, S-adenosyl methionine, chloramphenicol, and large biomolecules, e.g., thrombin, Ku, DNA polymerases, are effective targets for aptamers, catalytic RNAs (ribozymes) discussed herein (e.g., hammerhead RNAs, hairpin RNAs) as well as NASMs.

In preferred embodiments, the aptamers and NASMs of the invention specifically recognize components of ingestible solutions. The nucleic acids of the invention are therefore useful in the detection of targets such as caffeine and aspartame present in coffee and regular and diet soft drinks.

While the aptamer selection processes described identifies aptamers through affinity-based (binding) selections, the selection processes as described for NASMs identifies nucleic acid sensor molecules through target modulation of the catalytic core of a ribozyme. In NASM selection, selective pressure on the starting population of NASMs (starting pool size is as high as 10¹⁴ to 10¹⁷ molecules) results in nucleic acid sensor molecules with enhanced catalytic properties, but not necessarily in enhanced binding properties. Specifically, the NASM selection procedures place selective pressure on catalytic effectiveness of potential NASMS by modulating both target concentration and reaction time-dependence. Either parameter, when optimized throughout the selection, can lead to nucleic acid molecular sensor molecules which have custom-designed catalytic properties, e.g., NASMs that have high switch factors, and or NASMs that have high specificity.

II. Selection and Generation of a Target Specific Nucleic Acid Aptamer

Systematic Evolution of Ligands by Exponential Enrichment, “SELEX™,” is a method for making a nucleic acid ligand for any desired target, as described, e.g., in U.S. Pat. Nos. 5,475,096 and 5,270,163, and PCT/US91/04078, each of which is specifically incorporated herein by reference.

SELEX™ technology is based on the fact that nucleic acids have sufficient capacity for forming a variety of two- and three-dimensional structures and sufficient chemical versatility available within their monomers to act as ligands (i.e., form specific binding pairs) with virtually any chemical compound, whether large or small in size.

The method involves selection from a mixture of candidates and step-wise iterations of structural improvement, using the same general selection theme, to achieve virtually any desired criterion of binding affinity and selectivity. Starting from a mixture of nucleic acids, preferably comprising a segment of randomized sequence, the SELEX™ method includes steps of contacting the mixture with the target under conditions favorable for binding, partitioning unbound nucleic acids from those nucleic acids which have bound to target molecules, dissociating the nucleic acid-target pairs, amplifying the nucleic acids dissociated from the nucleic acid-target pairs to yield a ligand-enriched mixture of nucleic acids, then reiterating the steps of binding, partitioning, dissociating and amplifying through as many cycles as desired.

Within a nucleic acid mixture containing a large number of possible sequences and structures, there is a wide range of binding affinities for a given target. A nucleic acid mixture comprising, for example a 20 nucleotide randomized segment can have 420 candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands.

Cycles of selection and amplification are repeated until a desired goal is achieved. In the most general case, selection/amplification is continued until no significant improvement in binding strength is achieved on repetition of the cycle. The method may be used to sample as many as about 10¹⁸ different nucleic acid species. The nucleic acids of the test mixture preferably include a randomized sequence portion as well as conserved sequences necessary for efficient amplification. Nucleic acid sequence variants can be produced in a number of ways including synthesis of randomized nucleic acid sequences and size selection from randomly cleaved cellular nucleic acids. The variable sequence portion may contain fully or partially random sequence; it may also contain subportions of conserved sequence incorporated with randomized sequence. Sequence variation in test nucleic acids can be introduced or increased by mutagenesis before or during the selection/amplification iterations.

In one embodiment of SELEX™, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEX™ until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX™ process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX procedures with contiguous randomized segments be initiated with nucleic acid sequences containing a randomized segment of between about 20-50 nucleotides.

The basic SELEX™ method has been modified to achieve a number of specific objectives. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes a SELEX™ based methods for selecting nucleic acid ligands containing photoreactive groups capable of binding and/or photocrosslinking to and/or photoinactivating a target molecule. U.S. Pat. No. 5,567,588 and U.S. application Ser. No. 08/792,075, filed Jan. 31, 1997, entitled “Flow Cell SELEX”, describe SELEX™ based methods which achieve highly efficient partitioning between oligonucleotides having high and low affinity for a target molecule. U.S. Pat. No. 5,496,938 describes methods for obtaining improved nucleic acid ligands after the SELEX™ process has been performed. U.S. Pat. No. 5,705,337 describes methods for covalently linking a ligand to its target. Each of these patents and applications is specifically incorporated herein by reference.

SELEX™ can also be used to obtain nucleic acid ligands that bind to more than one site on the target molecule, and to nucleic acid ligands that include non-nucleic acid species that bind to specific sites on the target. SELEX™ provides means for isolating and identifying nucleic acid ligands which bind to any envisionable target, including large and small biomolecules including proteins (including both nucleic acid-binding proteins and proteins not known to bind nucleic acids as part of their biological function) cofactors and other small molecules. See U.S. Pat. No. 5,580,737 for a discussion of nucleic acid sequences identified through SELEX™ which are capable of binding with high affinity to caffeine and the closely related analog, theophylline.

Counter-SELEX™ is a method for improving the specificity of nucleic acid ligands to a target molecule by eliminating nucleic acid ligand sequences with cross-reactivity to one or more non-target molecules. Counter-SELEX™ is comprised of the steps of a) preparing a candidate mixture of nucleic acids; b) contacting the candidate mixture with the target, wherein nucleic acids having an increased affinity to the target relative to the candidate mixture may be partitioned from the remainder of the candidate mixture; c) partitioning the increased affinity nucleic acids from the remainder of the candidate mixture; d) contacting the increased affinity nucleic acids with one or more non-target molecules such that nucleic acid ligands with specific affinity for the non-target molecule(s) are removed; and e) amplifying the nucleic acids with specific affinity to the target molecule to yield a mixture of nucleic acids enriched for nucleic acid sequences with a relatively higher affinity and specificity for binding to the target molecule.

For example, a heterogeneous population of oligonucleotide molecules comprising randomized sequences is generated and selected to identify a nucleic acid molecule having a binding affinity which is selective for a target molecule. (U.S. Pat. Nos. 5,475,096; 5,476,766; and 5,496,938) each of is incorporated herein by reference. In some examples, a population of 100% random oligonucleotides is screened. In others, each oligonucleotide in the population comprises a random sequence and at least one fixed sequence at its 5′ and/or 3′ end. The oligonucleotide can be RNA, DNA, or mixed RNA/DNA, and can include modified or normatural nucleotides or nucleotide analogs. (U.S. Pat. Nos. 5,958,691; 5,660,985; 5,958,691; 5,698,687; 5,817,635; and 5,672,695, PCT publication WO 92/07065).

The random sequence portion of the oligonucleotide is flanked by at least one fixed sequence which comprises a sequence shared by all the molecules of the oligonucleotide population. Fixed sequences include sequences such as hybridization sites for PCR primers, promoter sequences for RNA polymerases (e.g., T3, T4, T7, SP6, and the like), restriction sites, or homopolymeric sequences, such as poly A or poly T tracts, catalytic cores (described further below), sites for selective binding to affinity columns, and other sequences to facilitate cloning and/or sequencing of an oligonucleotide of interest.

In one embodiment, the random sequence portion of the oligonucleotide is about 15-70 (e.g., about 30-40) nucleotides in length and can comprise ribonucleotides and/or deoxyribonucleotides. Random oligonucleotides can be synthesized from phosphodiester-linked nucleotides using solid phase oligonucleotide synthesis techniques well known in the art (Froehler et al., Nucl. Acid Res. 14:5399-5467 (1986); Froehler et al., Tet. Lett. 27:5575-5578 (1986)). Oligonucleotides can also be synthesized using solution phase methods such as triester synthesis methods (Sood et al., Nucl. Acid Res. 4:2557 (1977); Hirose et al., Tet. Lett., 28:2449 (1978)). Typical syntheses carried out on automated DNA synthesis equipment yield 10¹⁵-10¹⁷ molecules. Sufficiently large regions of random sequence in the sequence design increases the likelihood that each synthesized molecule is likely to represent a unique sequence.

To synthesize randomized sequences, mixtures of all four nucleotides are added at each nucleotide addition step during the synthesis process, allowing for random incorporation of nucleotides. In one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

The SELEX method encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. SELEX-identified nucleic acid ligands containing modified nucleotides are described in U.S. Pat. No. 5,660,985, which describes oligonucleotides containing nucleotide derivatives chemically modified at the 5′ and 2′ positions of pyrimidines. U.S. Pat. No. 5,756,703 describes oligonucleotides containing various 2′-modified pyrimidines. U.S. Pat. No. 5,580,737 describes highly specific nucleic acid ligands containing one or more nucleotides modified with 2′-amino (2′-NH₂), 2′-fluoro (2′-F), and/or 2′-O-methyl (2′-OMe) substituents.

The SELEX method encompasses combining selected oligonucleotides with other selected oligonucleotides and non-oligonucleotide functional units as described in U.S. Pat. No. 5,637,459 and U.S. Pat. No. 683,867. The SELEX method further encompasses combining selected nucleic acid ligands with lipophilic or non-immunogenic high molecular weight compounds in a diagnostic or therapeutic complex, as described in U.S. Pat. No. 6,011,020. VEGF nucleic acid ligands that are associated with a lipophilic compound, such as diacyl glycerol or dialkyl glycerol, in a diagnostic or therapeutic complex are described in U.S. Pat. No. 5,859,228.

VEGF nucleic acid ligands that are associated with a lipophilic compound, such as a glycerol lipid, or a non-immunogenic high molecular weight compound, such as polyalkylene glycol are further described in U.S. Pat. No. 6,051,698. VEGF nucleic acid ligands that are associated with a non-immunogenic, high molecular weight compound or a lipophilic compound are further described in PCT Publication No. WO 98/18480. These patents and applications allow the combination of a broad array of shapes and other properties, and the efficient amplification and replication properties, of oligonucleotides with the desirable properties of other molecules. Each of the above references, which describe modifications of the basic SELEX procedure are specifically incorporated by reference in its entirety.

The identification of nucleic acid ligands to small, flexible peptides via the SELEX method has been explored. Small peptides have flexible structures and usually exist in solution in an equilibrium of multiple conformers, and thus it was initially thought that binding affinities may be limited by the conformational entropy lost upon binding a flexible peptide. However, the feasibility of identifying nucleic acid ligands to small peptides in solution was demonstrated in U.S. Pat. No. 5,648,214. In this patent, high affinity RNA nucleic acid ligands to substance P, an 11 amino acid peptide, were identified. This reference is specifically incorporated by reference in its entirety.

To generate oligonucleotide populations which are resistant to nucleases and hydrolysis, modified oligonucleotides can be used and can include one or more substitute internucleotide linkages, altered sugars, altered bases, or combinations thereof. In one embodiment, oligonucleotides are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotide through an —O—, —N—, or —S— linkage. Not all linkages in the oligonucleotide are required to be identical.

In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). The use of 2-fluoro-ribonucleotide oligomer molecules can increase the sensitivity of a nucleic acid sensor molecule for a target molecule by ten-to-one hundred-fold over those generated using unsubstituted ribo- or deoxyribooligonucleotides (Pagratis, et al., Nat. Biotechnol. 15:68-73 (1997)), providing additional binding interactions with a target molecule and increasing the stability of the secondary structure(s) of the nucleic acid sensor molecule (Kraus, et al., Journal of Immunology 160:5209-5212 (1998); Pieken, et al., Science 253:314-317 (1991); Lin, et al., Nucl. Acids Res. 22:5529-5234 (1994); Jellinek, et al. Biochemistry 34:11363-11372 (1995); Pagratis, et al., Nat. Biotechnol 15:68-73 (1997)).

Nucleic acid aptamer molecules are generally selected in a 5 to 20 cycle procedure. In one embodiment, heterogeneity is introduced only in the initial selection stages and does not occur throughout the replicating process.

The starting library of DNA sequences is generated by automated chemical synthesis on a DNA synthesizer. This library of sequences is transcribed in vitro into RNA using T7 RNA polymerase and purified. In one example, the 5′-fixed:random:3′-fixed sequence is separated by a random sequence having 30 to 50 nucleotides.

1) Aptamers

Sorting among the billions of aptamer candidates to find the desired molecules starts from the complex sequence pool, whereby desired aptamers are isolated through an iterative in vitro selection process. The selection process removes both non-specific and non-binding types of contaminants. In a following amplification stage, thousands of copies of the surviving sequences are generated to enable the next round of selection. During amplification, random mutations can be introduced into the copied molecules-this ‘genetic noise’ allows functional nucleic acid aptamer molecules to continuously evolve and become even better adapted. The entire experiment reduces the pool complexity from 10¹⁷ molecules down to around 100 aptamer candidates that require detailed characterization.

Aptamer selection is accomplished by passing a solution of oligonucleotides through a column containing the target molecule (e.g., caffeine or aspartame). The flow-through, containing molecules which are incapable of binding target, is discarded. The column is washed, and the wash solution is discarded. Oligonucleotides which bound to the column are then specifically eluted, reverse transcribed, amplified by PCR (or other suitable amplification techniques), transcribed into RNA, and then reapplied to the selection column. Successive rounds of column application are performed until a pool of aptamers enriched in target binders is obtained.

Negative selection steps can also be performed during the selection process. Addition of such selection steps is useful to remove aptamers which bind to a target in addition to the desired target. Additionally, where the target column is known to contain an impurity, negative selection steps can be performed to remove from the binding pool those aptamers which bind selectively to the impurity, or to both the impurity and the desired target. For example, where the desired target is caffeine, care must be taken so as to remove aptamers which bind to closely related molecules such as theophylline. Examples of negative selection steps include, for example, incorporating column washing steps with theophylline in the buffer, or the addition of a theophylline column before the caffeine selection column (e.g., the flow through from the theophylline column will contain aptamers which do not bind theophylline).

After the completion of selection, the target-specific aptamers were reverse transcribed into DNA, cloned and amplified.

2) Uses of Aptamers

The typical process by which compounds present in a test mixture are identified is a high throughput screen. A high throughput screen is typically an assay configured to produce a detectible signal that is correlated to the presence or concentration of a component of the mixture. Samples whose detectible signal is unchanged relative to control samples without target do not contain the assayed compound and are called “misses”. Samples whose detectible signal is significantly changed relative to control samples without target, contain the assayed compound and are called “hits”.

Because the process of high throughput screening requires thousands to millions of assays, each assay will ideally be very reliable to prevent both false hits and false misses. The assay should also require minimal manipulation and additional reagents to keep the cost per assay as low as possible.

To facilitate use of the aptamers in high throughput screening assays, an aptamer can be generated with a 3′ sequence tag which specifically hybridizes with a biotinylated capture oligo. Such a capture oligo then can be used to immobilize the aptamer on a streptavidin coated substrate through the biotin-streptavidin binding. When such a streptavidin coated substrate is a flash plate (e.g., a plate containing a scintallant imbedded therein), surface immobilized aptamer RNA that binds to ³H-target will concentrate the tritiated nucleotide on the surface of the flash plate and generate a detectable scintillation proximity signal.

Using this methodology, aptamers can be analyzed for the ability to yield target-mediated signal in the SPA. Additionally, the aptamers can be analyzed for the ability to discriminate between target and closely related structural analogs.

III. Selection and Generation of a Target Specific-Nucleic Acid Sensor Molecule

1) Generation and selection of NASMs

Nucleic acid-based detection schemes have exploited the ligand-sensitive catalytic properties of some nucleic acids, e.g., such as ribozymes. Ribozyme-based nucleic acid sensor molecules have been designed both by engineering and by in vitro selection methods. Some engineering methods exploit the modular nature of nucleic acid structures by coupling molecular recognition to signaling by simply joining individual target-modulation and catalytic domains using, e.g., a double-stranded or partially double-stranded linker. ATP sensors, for example, have been created by appending the previously-selected, ATP-selective TMD sequences (see, e.g., Sassanfar et al., Nature 363:550-553 (1993)) to either the self-cleaving hammerhead ribozyme (see, e.g., Tang et al., Chem. Biol. 4:453-459 (1997)) as a hammerhead-derived sensor, or the L1 self-ligating ribozyme (see, e.g., Robertson et al., Nucleic Acids Res. 28:1751-1759 (2000)) as a ligase-derived sensor. Hairpin-derived sensors are also contemplated. In general, the target modulation domain is defined by the minimum number of nucleotides sufficient to create a three-dimensional structure which recognizes a target molecule.

Catalytic nucleic acid sensor molecules (NASMs) are selected which have a target molecule-sensitive catalytic activity (e.g., self-cleavage) from a pool of randomized or partially randomized oligonucleotides. The catalytic NASMs have a target modulation domain which recognizes the target molecule and a catalytic domain for mediating a catalytic reaction induced by the target modulation domain's recognition of the target molecule. Recognition of a target molecule by the target modulation domain triggers a conformational change and/or change in catalytic activity in the nucleic acid sensor molecule. In one embodiment, by modifying (e.g., removing) at least a portion of the catalytic domain and coupling it to an optical signal generating unit, an optical nucleic acid sensor molecule is generated whose optical properties change upon recognition of the target molecule by the target modulation domain. In one embodiment, the pool of randomized oligonucleotides comprises the catalytic site of a ribozyme.

A heterogeneous population of oligonucleotide molecules comprising randomized sequences is screened to identify a nucleic acid sensor molecule having a catalytic activity which is modified (e.g., activated) upon interaction with a target molecule. As with the aptamer nucleic acids, the oligonucleotide can be RNA, DNA, or mixed RNA/DNA, and can include modified or normatural nucleotides or nucleotide analogs.

Each oligonucleotide in the population comprises a random sequence and at least one fixed sequence at its 5′ and/or 3′ end. In one embodiment, the population comprises oligonucleotides which include as fixed sequences an aptamer known to specifically bind a particular target and a catalytic ribozyme or the catalytic site of a ribozyme, linked by a randomized oligonucleotide sequence. In a preferred embodiment, the fixed sequence comprises at least a portion of a catalytic site of an oligonucleotide molecule (e.g., a ribozyme) capable of catalyzing a chemical reaction.

Catalytic sites are well known in the art and include, e.g., the catalytic core of a hammerhead ribozyme (see, e.g., U.S. Pat. No. 5,767,263; U.S. Pat. No. 5,700,923) or a hairpin ribozyme (see, e.g., U.S. Pat. No. 5,631,359). Other catalytic sites are disclosed in U.S. Pat. No. 6,063,566; Koizumi et al., FEBS Lett. 239: 285-288 (1988); Haseloff and Gerlach, Nature 334: 585-59 (1988); Hampel and Tritz, Biochemistry 28: 4929-4933 (1989); Uhlenbeck, Nature 328: 596-600 (1987); and Fedor and Ublenbeck, Proc. Natl. Acad. Sci. USA 87: 1668-1672 (1990).

In some embodiments, a population of partially randomized oligonucleotides is generated from known aptamer and ribozyme sequences joined by the randomized oligonucleotides. Most molecules in this pool are non-functional, but a handful will respond to a given target and be useful as nucleic acid sensor molecules. Catalytic NASMs are isolated by the iterative process described above. In all embodiments, during amplification, random mutations can be introduced into the copied molecules this ‘genetic noise’ allows functional NASMs to continuously evolve and become even better adapted as target-activated molecules.

In another embodiment, the population comprises oligonucleotides which include a randomized oligonucleotide linked to a fixed sequence which is a catalytic ribozyme, the catalytic site of a ribozyme or at least a portion of a catalytic site of an oligonucleotide molecule (e.g., a ribozyme) capable of catalyzing a chemical reaction. The starting population of oligonucleotides is then screened in multiple rounds (or cycles) of selection for those molecules exhibiting catalytic activity or enhanced catalytic activity upon recognition of the target molecule as compared to the activity in the presence of other molecules, or in the absence of the target.

The nucleic acid sensor molecules identified through in vitro selection, e.g., as described above, comprise a catalytic domain (i.e., a signal generating moiety), coupled to a target modulation domain, (i.e., a domain which recognizes a target molecule and which transduces that molecular recognition event into the generation of a detectable signal). In addition, the nucleic acid sensor molecules of the present invention use the energy of molecular recognition to modulate the catalytic or confoniational properties of the nucleic acid sensor molecule.

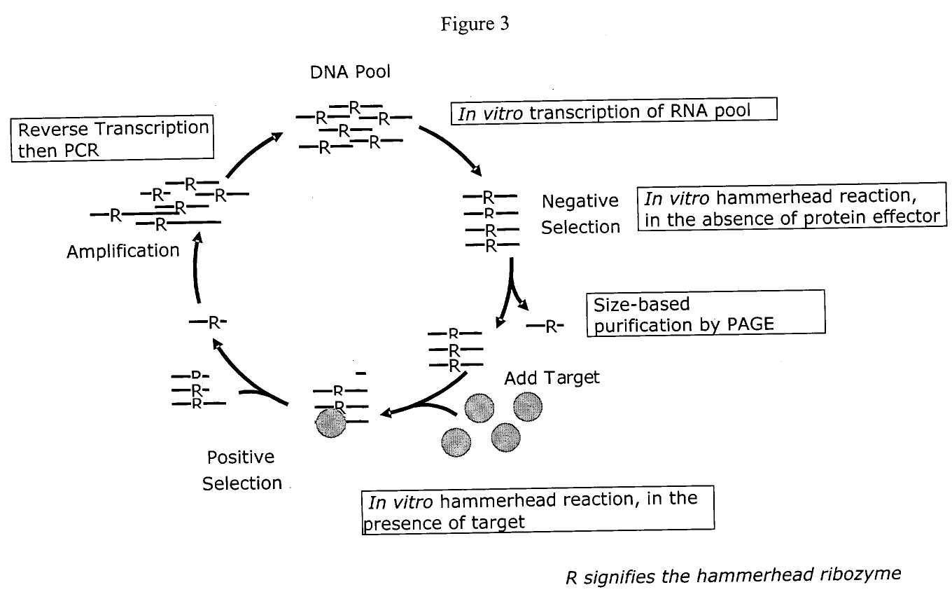

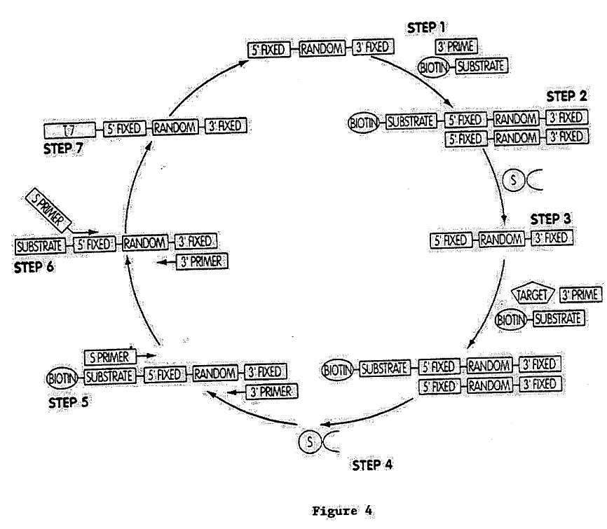

Nucleic acid sensor molecules are generally selected in a 5 to 20 cycle procedure. In one embodiment, heterogeneity is introduced only in the initial selection stages and does not occur throughout the replicating process. FIG. 4 shows a schematic diagram in which the oligonucleofide population is screened for a nucleic acid sensor molecule which comprises a target molecule activatable ligase activity. FIG. 3 shows the hammerhead nucleic acid sensor molecule selection methodology. Each of these methods are readily modified for the selection of NASMs with other catalytic activities.

Additional procedures may be incorporated in the various selection schemes, including: pre-screening, negative selection, etc. For example, individual clones isolated from selection experiments are tested early for allosteric activation in the presence of target-depleted extracts as a pre-screen, and molecules that respond to endogenous non-specific activators are eliminated from further consideration as target-modulated NASMs; to the extent that all isolated NASMs are activated by target-depleted extracts, depleted extracts are included in a negative selection step of the selection process; commercially available RNase inhibitors and competing RNase substrates (e.g. tRNA) may added to test samples to inhibit nucleases; or by carrying out selection in the presence of nucleases (e.g. by including depleted extracts during a negative selection step) the experiment intrinsically favors those molecules that are resistant to degradation; covalent modifications to RNA that can render it highly nuclease-resistant can be performed (e.g., 2′-O-methylation) to minimize non-specific cleavage in the presence of biological samples (see, e.g., Usman et al. Clin. Invest. 106:1197-202 (2000)).

In one embodiment, nucleic acid sensor molecules are selected which are activated by target molecules comprising molecules having an identified biological activity (e.g., a known enzymatic activity, receptor activity, or a known structural role); however, in another embodiment, the biological activity of at least one of the target molecules is unknown (e.g., the target molecule is a polypeptide expressed from the open reading frame of an EST sequence, or is an uncharacterized polypeptide synthesized based on a predicted open reading frame, or is a purified or semi-purified protein whose function is unknown).

Although in one embodiment the target molecule does not naturally bind to nucleic acids, in another embodiment, the target molecule does bind in a sequence specific or non-specific manner to a nucleic acid sensor molecule. In a further embodiment, a plurality of target molecules binds to the nucleic acid sensor molecule. Selection for NASMs specifically responsive to a plurality of target molecules (i.e. not activated by single targets within the plurality) may be achieved by including at least two negative selection steps in which subsets of the target molecules are provided. Nucleic acid sensor molecules can be selected which bind specifically to a modified target molecule but which do not bind to closely related target molecules. Stereochemically distinct species of a molecules can also be targeted.

A Target Modulation Domain with Endonucleolytic Activity

FIG. 3 shows the hammerhead nucleic acid sensor molecule selection methodology. As shown in FIG. 3, selection of an endonucleolytic nucleic acid sensor molecule (e.g., a hammerhead-derived NASM) begins with the synthesis of a ribozyme sequence on a DNA synthesizer. Random nucleotides are incorporated generating pools of roughly 10¹⁶ molecules. Most molecules in this pool are non-functional, but a handful will respond to a given target and be useful as nucleic acid sensor molecules. Sorting among the billions of species to find the desired molecules starts from the complex sequence pool. Nucleic acid sensor molecule are isolated by an iterative process: in addition to the target-activated ribozymes that one desires, the starting pool is usually dominated by either constitutively active or completely inactive ribozymes. The selection process removes both types of contaminants by incorporating both negative and positive selection incubation steps. In the following amplification stage, thousands of copies of the surviving sequences are generated to enable the next round of selection. During amplification, random mutations can be introduced into the copied molecules this ‘genetic noise’ allows functional NASMs to continuously evolve and become even better adapted as target-activated molecules. The entire experiment reduces the pool complexity from 10¹⁶ down to <100.

The starting library of DNA sequences (the “Pool”) is generated by automated chemical synthesis on a DNA synthesizer. This library of sequences is transcribed in vitro into RNA using T7 RNA polymerase and subsequently purified. In the absence of the desired target molecule of interest, the RNA library is incubated together with the binding buffer alone as a negative selection incubation. During this incubation, non-allosteric (or non-target activated) ribozymes are expected to undergo a catalytic reaction, in this case, cleavage. Undesired members of the hammerhead pool, those that are constitutively active in the absence of the target molecule, are removed from the unreacted members by size-based purification, e.g., by PAGE-chromatography; 7 M Urea, 8-10% acrylamide, 1×TBE. Higher molecular weight species are eluted as a single broad band from the gel matrix into TBE buffer, then purified for subsequent steps in the selection cycle. The remaining RNA pool is then incubated under identical conditions but now in the presence of the target molecule of interest in binding buffer, as a positive selection incubation. In another size-based purification, desired members of the hammerhead pool, those that are only active in the presence of the target molecule, are removed from the remaining unreacted members by PAGE-chromatography; 7 M Urea, 8-10% acrylamide, 1×TBE. In this step, lower molecular weight species are eluted as a single broad band from the gel matrix into TBE buffer, then purified for subsequent steps in the selection cycle. RT-PCR amplified DNA is then purified and transcribed to yield an enriched pool for a subsequent round of reselection. Rounds of selection and amplification are repeated until functional members sufficiently dominate the resultant library.

B. Target Modulation Domain with Ligase Activity

FIG. 4 shows a schematic diagram in which the oligonucleotide population is screened for a nucleic acid sensor molecule which comprises a target molecule activatable ligase activity. In the embodiment shown in FIG. 4, the ligation reaction involves covalent attachment of an oligonucleotide substrate to the 5′-end of the NASM through formation of a phosphodiester linkage. Other ligation chemistries can form the basis for selection of NASMs (e.g., oligonucleotide ligation to the 3′-end, alkylations (see, e.g., Wilson et al., Nature 374(6525):777-782 (1995)), peptide bond formation (see, e.g., Zhang et al., Nature 390(6655):96-100 (1997)), Diels-Alder reactions to couple alkenes and dienes (see, e.g., Seelig et al., Chemistry and Biology 3:167-176 (1999)). For some chemistries, the chemical functional groups that constitute the reactants in the ligation reaction may not naturally appear within nucleic acids. Thus, it may be necessary to synthesize an RNA pool in which one of the ligation reactants is covalently attached to each member of the pool (e.g., attaching a primary amine to the 5′-end of an RNA to enable selection for peptide bond formation).

In this embodiment, the oligonucleotide population from which the NASMs are selected is initially screened in a negative selection procedure to eliminate any molecules which have ligase activity even in the absence of target molecule binding. A solution of oligonucleotides (e.g., 100 pM) comprising a 5′ and 3′ fixed sequence (“5”-fixed: random: 3“-fixed”) is denatured with a 3′ primer sequence (“3′ prime”) (e.g., 200 pM) which binds to at least a portion of the 3′ fixed sequence. Ligation buffer (e.g., 30 mM Tris HCl, pH 7.4, 600 mM NaCl, 1 mM EDTA, 1% NP-40, 60 mM MgCl₂) and a tagged oligonucleotide substrate sequence (“tag-substrate”) (e.g., Tag-UGCCACU) are added and the mixture is incubated for about 16 to about 24 hours at 25° C. in the absence of target molecule (STEP 1). Tags encompassed within the scope include, e.g., radioactive labels, fluorescent labels, a chemically reactive species such as thiophosphate, the first member of a binding pair comprising a first and second binding member, each member bindable to the other (e.g., biotin, an antigen recognized by an antibody, or a tag nucleic acid sequence). The reaction is stopped by the addition of EDTA. Alternatively, the reaction can be terminated by removal of the substrate or addition of denaturants (e.g., urea or formamide).

Ligated molecules are removed from pool of selectable molecules (STEP 2), generating a population of oligonucleotides substantially free of ligated molecules (as measured by absence of the tag sequence in the solution). In the embodiment shown in FIG. 4, the tag is the first member of a binding pair (e.g., biotin) and the ligated molecules (“biotin-oligonucleotide substrate:5′-fixed:random:3′-fixed”) are physically removed from the solution by contacting the sample to a solid support to which the second member of the binding pair is bound (“S”) (e.g., streptavidin). The eluant collected comprises a population of oligonucleotides enriched for non-ligated molecules (5′-fixed:random:3′-fixed). This step can be repeated multiple times until the oligonucleotide population is substantially free of molecules having target-insensitive ligase activity.

This step allows for suppression of the ability of constitutively active molecules to be carried through to the next cycle of selection. Physical separation of ligated and unligated molecules is one mechanism by which this can be achieved. Alternatively, the negative selection step can be configured such that catalysis converts active molecules to a form that blocks their ability to be either retained during the subsequent positive selection step or to be amplified for the next cycle of selection. For example, the oligonucleotide substrate used for ligation in the negative selection step can be synthesized without a capture tag. Target-independent ligases covalently self-attach the untagged oligonucleotide substrate during the negative selection step and are then unable to accept a tagged form of the oligonucleotide substrate provided during the positive selection step that follows. In another embodiment, the oligonucleotide substrate provided during the negative selection step has a different sequence from that provided during the positive selection step. When PCR is carried out using a primer complementary to the positive selection oligonucleotide substrate, only target-activated ligases will be capable of amplification.

A positive selection phase follows. In this phase, more 3′ primer and tagged oligonucleotide substrate are added to the pool resulting from the negative selection step. Target molecules are then added to form a reacted solution and the reacted solution is incubated at 25° C. for about 2 hours (STEP 3). Target molecules encompassed within the scope include, e.g., proteins or portions thereof (e.g., receptors, antigen, antibodies, enzymes, growth factors), peptides, enzyme inhibitors, hormones, carbohydrates, polysaccharides, glycoproteins, lipids, phospholipids, metabolites, metal ions, cofactors, inhibitors, drugs, dyes, vitamins, nucleic acids, membrane structures, receptors, organelles, and viruses. Target molecules can be free in solution or can be part of a larger cellular structure (e.g., such as a receptor embedded in a cell membrane). In one embodiment, a target molecule is one which does not naturally bind to nucleic acids.

The reacted solution is enriched for ligated molecules (biotin-oligonucleotide substrate: 5′-fixed:random:3′-fixed) by removing non-tagged molecules (5′-fixed:random:3′-fixed) from the solution. For example, in one embodiment, the tagged oligonucleotide substrate comprises a biotin tag and ligated molecules are isolated by passing the reacted solution over a solid support to which streptavidin (S) is bound (STEP 4). Eluant containing non-bound, non-ligated molecules (5′-fixed:random:3′-fixed) is discarded and bound, ligated molecules (biotin-oligonucleotide substrate: 5′-fixed:random:3′-fixed) are identified as nucleic acid sensor molecules and released from the support by disrupting the binding pair interaction which enabled capture of the catalytically active molecules. For example, heating to 95° C. in the presence of 10 mM biotin allows release of biotin-tagged catalysts from an immobilized streptavidin support. In another embodiment, the captured catalysts remain attached to a solid support and are directly amplified (described below) while immobilized. Multiple positive selection phases can be performed (STEPS 3 and 4). In one embodiment, the stringency of each positive selection phase is increased by decreasing the incubation time by one half.

Physically removing inactive species from the pool adds stringency to the selection process. However, to the extent that the ligation reaction increases the amplification potential of the NASMs, this step may be omitted. In the illustrated embodiment, for example, ligation of an oligonucleotide to the active species provides a primer binding site that enables subsequent PCR amplification using an oligonucleotide substrate complementary to the original oligonucleotide substrate. Unligated species do not necessarily need to be physically separated from other species because they are less likely to amplify in the absence of a covalently tethered primer binding site. Selected nucleic acid sensor molecules are amplified (or in the case of RNA molecules, first reverse transcribed, then amplified) using an oligonucleotide substrate primer (“S primer”) which specifically binds to the ligated oligonucleotide substrate sequence (STEP 5). In one embodiment, amplified molecules are further amplified with a nested PCR primer that regenerates a T7 promoter (“T7 Primer”) from the 5′ fixed and the litigated oligonucleotide substrate sequence (STEP 6). Following transcription with T7 RNA polymerase (STEP 7), the oligonucleotide pool may be further selected and amplified to eliminate any remaining unligated sequences (5′-fixed:random:3′-fixed) by repeating STEPS 3-7. It should be obvious to those of skill in the art that in addition to PCR, and RT-PCR, any number of amplification methods can be used (either enzymatic, chemical, or replication-based, e.g., such as by cloning), either singly, or in combination. Exemplary amplification methods are disclosed in Saiki, et al., Science 230:1350-1354 (1985); Saiki, et al., Science 239:481-491 (1988); Kwoh, et al., Proc. Natl. Acad. Sci. 86:1173 (1989); Joyce, Molecular Biology of RNA: UCLA Symposia on Molecular and Cellular Biology, T. R. Cech (ed.) pp. 361-371 (1989); and Guatelli, et al., Proc. Natl. Acad. Sci. 87:1874 (1990).

Because the 3′ primer (3′ prime) (see STEP 3 in FIG. 4) is included in the ligation mixture, selected nucleic acid sensor molecules may require this sequence for activation. In cases where this is undesirable, the 3′ primer may be omitted from the mix. Alternatively, the final nucleic acid sensor molecule can be modified by attaching the 3′ primer via a short sequence loop or a chemical linker to the 3′ end of the nucleic acid sensor molecule, thereby eliminating the requirement for added primer, allowing 3′ primer sequence to self-prime the molecule.

C. Target Modulation Domain with Self-Cleaving Activity

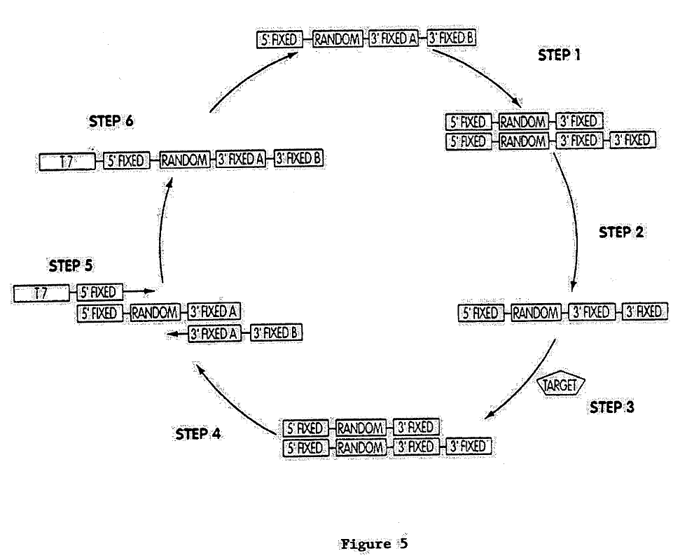

In another embodiment, as shown in FIG. 5, an oligonucleotide population is screened for a nucleic acid sensor molecule which comprises a target molecule having activatable self-cleaving activity. In this embodiment, the starting population of oligonucleotide molecules comprises 5′ and 3′ fixed regions (“5”-fixed and 3′ fixed A-3“fixed B”) and at least one of the fixed regions, in this example, the 3′ fixed region, comprises a ribozyme catalytic core including a self cleavage site (the junction between 3′ fixed A-3 ′fixed B). The population of oligonucleotide molecules comprising random oligonucleotides flanked by fixed 5′ and 3′ sequences (5′-fixed:random:3′-fixed A: 3′ fixed B) are negatively selected to remove oligonucleotides which self-cleave (i.e., 5′-fixed:random:3′-fixed-A molecules) even in the absence of target molecules. The oligonucleotide pool is incubated in reaction buffer (e.g., 50 mM Tris HCl, pH 7.5, 20 mM MgCl₂) for 5 hours at 25° C., punctuated at one hour intervals by incubation at 60° C. for one minute (STEP 1). In one embodiment, the uncleaved fraction of the oligonucleotide population (containing 5′-fixed and 3′ fixed A-3′-fixed B molecules) is purified by denaturing 10% polyacrylamide gel electrophoresis (PAGE) (STEP 2). Target molecule dependent cleavage activity is then selected in the presence of target molecules in the presence of reaction buffer by incubation at 23° C. for about 30 seconds to about five minutes (STEP 3). Cleaved molecules (5′-fixed:random:3′fixed-A molecules) are identified as nucleic acid sensor molecules and are purified by PAGE (STEP 4).

Amplification of the cleaved molecule is performed using primers which specifically bind the 5′-fixed and the 3′-fixed A sequences, regenerating the T7 promoter and the 3′-fixed B site (STEP 5), and the molecule is further amplified further by RNA transcription using T7 polymerase (STEP 6). In one embodiment, the process (STEPS 1-6) is repeated until the starting population is reduced to about one to five unique sequences.

Alternative methods for separating cleaved from uncleaved RNAs can be used. Tags can be attached to the 3′-fixed B sequence and separation can be based upon separating tagged sequences from non-tagged sequences at STEP 4. Chromatographic procedures that separate molecules on the basis of size (e.g., gel filtration) can be used in place of electrophoresis. One end of each molecule in the RNA pool can be attached to a solid support and catalytically active molecules isolated upon release from the support as a result of cleavage. Alternate catalytic cores may be used. These alternate catalytic cores and methods using these cores are also are encompassed within the scope of the invention.

D. Other Target Modulation Domains

Nucleic acid sensor molecules which utilize other catalytic activities or which combine both cleavage and ligase activities in a single molecule can be isolated by using one or a combination of both of the selection strategies outlined independently above for ligases and endonucleases. For example, the hairpin ribozyme is known to catalyze cleavage followed by ligation of a second oligonucleotide substrate (Berzal-Herranz et al., Genes and Development 1:129-134 (1992)). Target activated sensor molecules based on the hairpin activity can be isolated from a pool of randomized sequence RNAs. Hairpin-based NASMs can be isolated on the basis of target molecule dependent release of the fragment in the same way that hammerhead-based NASMs are isolated (e.g., target molecule dependent increase in electrophoretic mobility or target molecule dependent release from a solid support). Alternatively, nucleic acid sensor molecules can be selected on the basis of their ability to substitute the 3′-sequence released upon cleavage for another sequence as described in an target molecule independent manner by Berzal-Herranz et al., Genes and Development 1:129-134 (1992). In this scheme, the original 3′-end of the NASM is released in an initial cleavage event and an exogenously provided oligonucleotide substrate with a free 5′-hydroxyl is ligated back on. The newly attached 3′-end provides a primer binding site that can form the basis for preferential amplification of catalytically active molecules. Constitutively active molecules that are not activated by a provided target molecule can be removed from the pool by (1) separating away molecules that exhibit increased electrophoretic mobility in the absence of an exogenous oligonucleotide substrate or in the absence of target molecule, or (2) capturing molecules that acquire an exogenous oligonucleotide substrate (e.g., using a 3′-biotinylated substrate and captured re-ligated species on an avidin column).

Like the hairpin ribozyme, the group I intron self-splicing ribozymes combine cleavage and ligation activities to promote ligation of the exons that flank it. In the first step of group I intron-catalyzed splicing, an exogenous guanosine cofactor attacks the 5′-splice site. As a result of an intron-mediated phosphodiester exchange reaction, the 5′-exon is released coincident with attachment of the guanosine cofactor to the ribozyme. In a second chemical step, the 3′-hydroxyl at the end of the 5′-exon attacks the phosphodiester linkage between the intron and the 3′-exon, leading to ligation of the two exons and release of the intron. Group I intron-derived NASMs can be isolated from degenerate sequence pools by selecting molecules on the basis of either one or both chemical steps, operating in either a forward or reverse direction. NASMs can be isolated by specifically enriching those molecules that fail to promote catalysis in the absence of target molecule but which are catalytically active in its presence. Specific examples of selection schemes follow. In each case, a pool of RNAs related in sequence to a representative group I intron (e.g., the Tetrahymena thermophila pre-rRNA intron or the phage T4 td intron) serves as the starting point for selection. Random sequence regions can be embedded within the intron at sites known to be important for proper folding and activity (e.g., substituting the P5abc domain of the Tetrahymena intron, Williams et al., Nucl. Acid Res. 22(11):2003-2009 (1994)). Intron nucleic acid sensor molecules, in this case, sensitive to thio-GMP can be generated as follows.

In the first step, forward direction, the intron is synthesized with a short 5′-exon. In the negative selection step, a guanosine cofactor is provided and constitutively active molecules undergo splicing. In the positive selection step, the target molecule is provided together with thio-GMP. Molecules responsive to the target undergo activated splicing and as a result acquire a unique thiophosphate at their 5′-termini. Thio-tagged NASMs can be separated from untagged ribozymes by their specific retention on mercury gels or activated thiol agarose columns.

The first step, reverse direction method is performed as described in Green & Szostak. An intron is synthesized with a 5′-guanosine and no 5′-exon. An oligonucleotide substrate complementary to the 5′-internal guide sequence is provided during the negative selection step and constitutively active molecules ligate the substrate to their 5′-ends, releasing the original terminal guanosine. A second oligonucleotide substrate with a different 5′-sequence is provided together with target in the positive selection step. NASMs specifically activated by the target molecule ligate the second oligonucleotide substrate to their 5′-ends. PCR amplification using a primer corresponding to the second substrate can be carried out to preferentially amplify target molecule sensitive nucleic acid sensor molecules.

The second step, reverse direction method is performed as described in Nature 344:467-468 (1990). The intron is synthesized with no flanking exons. During the negative selection step, pool RNAs are incubated together with a short oligonucleotide substrate under conditions which allow catalysis to proceed. During the positive selection step, a second oligonucleotide substrate with a different 3′-sequence is provided together with the sensor target. NASMs are activated and catalyze ligation of the 3′-end of the second substrate. Reverse transcription carried out using a primer complementary to the 3′-end of the second substrate specifically selects NASMs for subsequent amplification.

2) Characterization of NASMs

Once particular aptamers or nucleic acid sensor molecules have been selected, they can be isolated, cloned, sequenced, and/or resynthesized using natural or modified nucleotides. Accordingly, synthesis intermediates of nucleic acid compositions are also encompassed within the scope of the invention, as are replicatable sequences (e.g., plasmids) comprising the nucleic acid compositions of the invention.

The pool of NASMs is cloned into various plasmids transformed, e.g., into E. coli. Individual NASM encoded DNA clones are isolated, PCR amplified and to generate NASM RNA. The NASM RNAs are then tested in target modulation assays which determine the rate or extent of ribozyme modulation. For hammerhead NASMs, the extent of target dependent and independent reaction is determined by quantifying the extent of endonucleolytic cleavage of an oligonucleotide substrate. The extent of reaction can be followed by electrophoresing the reaction products on a denaturing PAGE gel, and subsequently analyzed by standard radiometric methods. For ligase NASMs, the extent of target dependent and independent reaction is determined by quantifying the extent of ligation of an oligonucleotide substrate, resulting in an increase in NASM molecular weight, as determined in denaturing PAGE gel electrophoresis.

Individual NASM clones which display high target dependent switch factor values, or high k_{act} rate values are subsequently chosen for further modification and evaluation.

Hammerhead-derived NASM clones are then further modified to render them suitable for the optical detection applications that are described in detail below. These NASMs are used as fluorescent biosensors affixed to solid supports, as fluorescent biosensors in homogeneous (solution) FRET-based assays, and as biosensors in SPA applications.

Ligase and intron-derived NASM clones are further modified to render them suitable for a number of detection platforms and applications, including, but not limited to, PCR and nucleotide amplification detection methods; fluorescent-based biosensors detectable in solution and chip formats; and as in vivo, intracellular detection biosensors.

An important kinetic consideration in NASM characterization is the fact that RNAse-mediated degradation of the nucleic acid sensor molecule proceeds at a rate in competition with the rate of nucleic acid sensor molecule catalysis. As such, nucleic acid sensor molecules with fast turnover rates can be assayed for shorter times and are thus less susceptible to RNAse problems. Nucleic acid sensor molecules with fast turnover can be obtained by (1) reducing the length of the incubation during the positive selection step, and/or (2) choosing fast nucleic acid sensor molecules (potentially with less favorable allosteric activation ratios) when screening individual clones emerging from the selection experiment.

The relative stabilities of the activated and unactivated forms of the nucleic acid sensor molecules can be optimized to achieve the highest sensitivity of detection of target molecule. In one embodiment, the nucleic acid sensor molecule is further engineered to enhance the stability of one form over another, such as favoring the formation of the target molecule activated form. As in the case where certain bases do not form base pairs when the nucleic acid sensor molecule is unactivated, the unactivated form is not stabilized.

A number of methods can be used to evaluate the relative stability of different conformations of the nucleic acid sensor molecule. In one embodiment, the free energy of the structures formed by the nucleic acid sensor molecule is determined using software programs such as mfold®, which can be found on the Rensselaer Polytechnic Institute (RPI) web site.

In another embodiment, a gel assay is performed which permits detection of different conformations of the nucleic acid sensor molecule. In this embodiment, the nucleic acid sensor molecule is allowed to come to equilibrium at room temperature or the temperature at which the nucleic acid sensor molecule will be used. The molecule is then cooled to 4° C. and electrophoresed on a native (non-denaturing) gel at 4° C. Each of the conformations formed by the nucleic acid sensor molecule will run at a different position on the gel, allowing visualization of the relative concentration of each conformation. Similarly, the conformation of nucleic acid sensor molecules which form in the presence of target molecule is then determined by a method such as circular dichroism (CD). By comparing the conformation of the nucleic acid sensor molecule formed in the presence of target molecule with the conformations formed in the absence of target molecule, the conformation which corresponds to the activated conformation can be identified in a sample in which there is no target molecule. The nucleic acid sensor molecule can then be engineered to minimize the formation of the activated conformation in the absence of target molecule. The sensitivity and specificity of nucleic acid sensor molecule can be further tested using target molecule modulation assays with known amounts of target molecules.

Modifications to stabilize one conformation of the nucleic sensor molecule over another may be identified using the mfold program or native gel assays discussed above. A labeled nucleic acid sensor molecule is generated by coupling a first signaling moiety (F) to a first nucleotide and a second signaling moiety (D) to a second nucleotide as discussed above. As above, the sensitivity and specificity of the nucleic acid sensor molecule can be further assayed by using target molecule modulation assays with known amounts of target molecules.

3) Converting a Catalytic NASM to an Optical NASM

During or after synthesis of the NASM, an optical signal generating unit is either added or inserted into the oligonucleotide sequence comprising the derived nucleic acid sensor molecule. In one embodiment, in order to convert a catalytic nucleic acid sensor molecule into an optical nucleic acid sensor molecule, at least a portion of the catalytic domain is modified (e.g., deleted). In one embodiment, the deletion enhances the conformational stability of the optical nucleic acid sensor molecule in either the bound or unbound forms. In one embodiment, deletion of the entire catalytic domain of the catalytic NASM stabilizes the unbound form of the nucleic acid sensor molecule. In another embodiment, the deletion may be chosen so as to take advantage of the inherent fluorescence-quenching properties of unpaired guanosine (G) residues (Walter and Burke, RNA 3:392 (1997)).

In another embodiment, the target modulation domain from a previously identified nucleic acid sensor molecule is incorporated into an oligonucleotide sequence that changes conformation upon target recognition. Nucleic acid sensor molecules of this type can be derived from allosteric ribozymes, such as those derived from the hammerhead, hairpin, L1 ligase, or group 1 intron ribozymes and the like, all of which transduce molecular recognition into a detectable signal. For example, 3′,5′-cyclic nucleotide monophosphate (cNMP)-dependent hammerhead ribozymes were reengineered into (RNA) sensor molecules which specifically bound to cNMP (Soukup et al., RNA 7:524 (2001)). The catalytic cores for hammerhead ribozymes were removed and replaced with 5-base duplex forming sequences. The binding of these reengineered RNA sensor molecules to cNMP was then confirmed experimentally. By adjusting the duplex length, sensor molecules can be redesigned to undergo significant conformational changes. The conformational changes can then be coupled to detection via FRET or simply changes in fluorescence intensity (as in the case of a molecular beacon). For example, by adding an appropriate probe on each end of the duplex, the stabilization of duplex by target binding can be monitored with the change in fluorescence.

While the above experimental example is performed in solution and utilizes a cuvette-based fluorescence spectrometer, in alternative embodiments the methods are performed in microwell multiplate readers (e.g., the Packard Fusion, or the Tecan Ultra) for high-throughput solution phase measurements.

In one embodiment, after deletion of at least a portion of the catalytic site from a catalytic nucleic acid sensor molecule, an optical signaling unit is either added to, or inserted within, the nucleic sensor molecule, generating a sensor molecule whose optical properties change in response to binding of the target molecule to the target modulation domain. In one embodiment, the optical signaling unit is added by exposing at least a 5′ or 3′ nucleotide that was not previously exposed. The 5′ nucleotide or a 5′ subterminal nucleotide (e.g., an internal nucleotide) of the molecule is couplable to a first signaling moiety while the 3′ nucleotide or 3′ subterminal nucleotide is couplable to a second signaling moiety. Target molecule recognition by the optical nucleic acid sensor molecule alters the proximity of the 5′ and 3′ nucleotide (or subterminal nucleotides) with respect to each other, and when the first and second signaling moieties are coupled to their respective nucleotides, this change in proximity results in a target sensitive change in the optical properties of the nucleic acid sensor molecule. Detection of changes in the optical properties of the nucleic acid sensor molecule can therefore be correlated with the presence and/or quantity of a target molecule in a sample.

In another embodiment, optical NASMs are generated by adding first and second signaling moieties, that are coupled to the 5′ terminal or subterminal sequences, and 3′-l terminal and subterminal sequences respectively, of the catalytic NASM. Signaling molecules can be coupled to nucleotides which are already part of the nucleic acid sensor molecule or may be coupled to nucleotides which are inserted into the nucleic acid sensor molecule, or can be added to a nucleic acid sensor molecule as it is synthesized. Coupling chemistries to attach signaling molecules are well known in the art (see, e.g., The Molecular Probes Handbook, R. Haughland). Suitable chemistries include, e.g., derivatization of the 5-position of pyrimidine bases (e.g., using 5′-amino allyl precursors), derivatization of the 5′-end (e.g., phosphoroamidites that add a primary amine to the 5′-end of chemically-synthesized oligonucleotide) or the 3′-end (e.g., periodate treatment of RNA to convert the 3′-ribose into a dialdehyde which can subsequently react with hydrazide-bearing signaling molecules).

In another embodiment, a single signaling moiety is either added to, or inserted within, the catalytic nucleic sensor molecule. In this embodiment, binding of the target molecule results in changes in both the conformation and physical aspect (e.g., molecular volume, and thus rotational diffusion rate, etc.) of the optical nucleic acid sensor molecule. Conformational changes in the optical nucleic acid sensor molecule upon target recognition will modify the chemical environment of the signaling moiety, while changes in the physical aspect of the nucleic acid sensor molecule will alter the kinetic properties of the signaling moiety. In both cases, the result will be a detectable change in the optical properties of the nucleic acid sensor molecule.

In one embodiment, the optical nucleic acid sensor molecule is prepared without a quencher group. Instead of a quencher group, a moiety with a free amine group can be added. This free amine group allows the sensor molecule to be attached to an aldehyde-derivatized glass surface via standard protocols for Schiff base formation and reduction. The nucleic acid sensor molecules can be bound in discrete regions or spots to form an array, or uniformly distributed to cover an extended area. In the absence of target, the optical nucleic acid sensor molecule will diffusionally rotate about its point of attachment to the surface at a rate characteristic of its molecular volume and mass. After target recognition and modulation of the structure of the NASM, the optical NASM-target complex will have a correspondingly larger volume and mass. This change in molecular volume (mass) will slow the rate of rotational diffusion, and result in a measurable change in the polarization state of the fluorescence emission from the fluorophore.

In one embodiment of the invention, a single signaling moiety is attached to a portion of a catalytic NASM that is released as a result of catalysis (e.g., either end of a self-cleaving ribozyme or the pyrophosphate at the 5′-end of a ligase). Target molecule-activated catalysis leads to release of the signaling moiety from the optical NASM to generate a signal correlated with the presence of the target. Release can be detected by either (1) changes in the intrinsic optical properties of the signaling moiety (e.g., decreased fluorescence polarization as the released moiety is able to tumble more freely in solution), or (2) changes in the partitioning of the signaling moiety (e.g., release of a fluorophore from a chip containing immobilized ribozymes such that the total fluorescence of the chip is reduced following washing).

In another embodiment of the invention, the catalytic nucleic acid sensor molecule is unmodified and the optical signaling unit is provided as a substrate for the NASM. One example of this embodiment includes a fluorescently tagged oligonucleotide substrate which can be joined to a NASM with ligase activity. In a heterogeneous assay using the ligase as a sensor molecule, analyte-containing samples are incubated with the fluorescent oligonucleotide substrate and the ligase under conditions that allow the ligase to function. Following an incubation period, the ligase is separated from free oligonucleotide substrate (e.g., by capturing ligases onto a solid support on the basis of hybridization to ligase-specific sequences or by pre-immobilizing the ligases on a solid support and washing extensively).

Quantitation of the captured fluorescence signal provides a means for inferring the concentration of analyte in the sample. In a second example of this embodiment, catalytic activity alters the fluorescence properties of a oligonucleotide substrate without leading to its own modification. Fluorophore pairs or fluorophore/quencher pairs can be attached to nucleotides flanking either side of the cleavage site of an oligonucleotide substrate for a trans-acting endonuclease ribozyme (Jenne et al., Nature Biotechnology 19(1):56-61 (2001)). Target activated cleavage of the substrate leads to separation of the pair and a change in its optical properties.

In another embodiment of the invention, the ligase catalytic NASM and its oligonucleotide substrates are unmodified and detection relies on catalytically-coupled changes in the ability of the NASM to be enzymatically amplified. In one example, a target-activated ligase is incubated together with oligonucleotide substrate and an analyte-containing sample under conditions which allow the ligase to function. Following an incubation period, the reaction is quenched and the mixture subjected to RT/PCR amplification using a primer pair that includes the oligo sequence corresponding to the ligation substrate. Amplification products can be detected by a variety of generally practiced methods (e.g. Taqman®). Only those ribozymes that have self-ligated an oligonucleotide substrate are capable of amplification under these conditions and will generate a signal that can be coupled to the concentration of the sensor target.

4) Detection of optical NASMs

i) Proximity Dependent Signaling Moieties

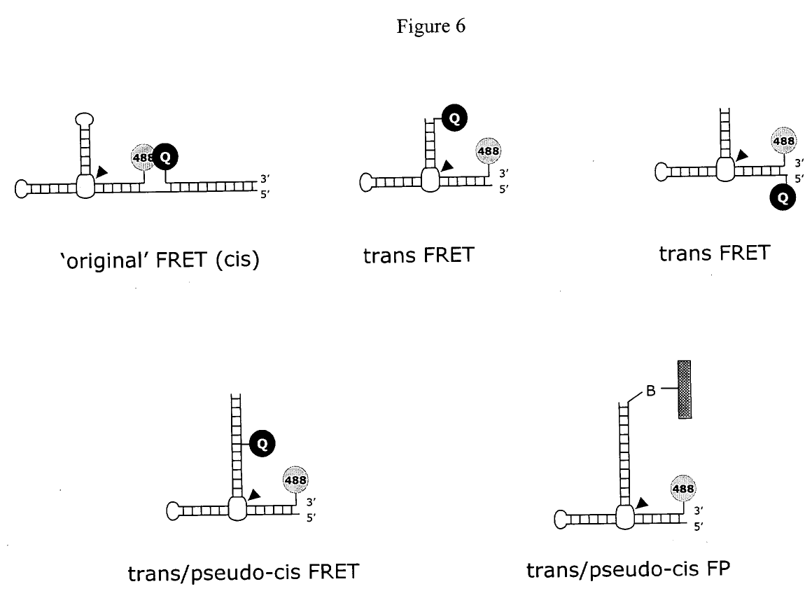

Many proximity dependent signaling moieties are known in the art and are encompassed within the scope of the present invention (Morrison, Nonisotopic DNA Probe Techniques, Kricka, ed., Academic Press, Inc., San Diego, Calif., chapter 13; Heller et al., Academic Press, Inc. pp. 245-256 (1985)). Systems using these signaling moieties rely on the change in fluorescence that occurs when the moieties are brought into close proximity. Such systems are described in the literature as fluorescence energy transfer (FET), fluorescence resonance energy transfer (FRET), nonradiative energy transfer, long-range energy transfer, dipole-coupled energy transfer, or Forster energy transfer (U.S. Pat. No. 5,491,063, Wu et al., Anal. Biochem. 218:1 (1994)). The arrangement of various fluorophore-quencher pairs is shown in FIG. 6. (See Jenne et al., Nature Biotechnology 1:56-61 (2001); Singh et al., RNA 5:1348 (1999); Frauendorf et al., Bioorg Med. Chem. 10:2521-2524 (2001); Perkins et al., Biochemistry 35(50):16370-16377 (1996)), and WO 99/47704 for discussion of various FRET formats.

Suitable fluorescent labels are known in the art and commercially available from, for example, Molecular Probes (Eugene, Oreg.). These include, e.g., donor/acceptor (i.e., first and second signaling moieties) molecules such as: fluorescein isothiocyanate (FITC)/tetramethylrhodamine isothiocyanate (TRITC), FITC/Texas Red), FITC/N-hydroxysuccinimidyl 1-pyrenebutyrate (PYB), FITC/eosin isothiocyanate (EITC), N-hydroxysuccinimidyl 1-pyrenesulfonate (PYS)/FITC, FITC/Rhodamine X (ROX), FITC/tetramethylrhodamine (TAMRA), and others. In addition to the organic fluorophores already mentioned, various types of nonorganic fluorescent labels are known in the art and are commercially available from, for example, Quantum Dot Corporation, Inc. (Hayward, Calif.). These include, e.g., donor/acceptor (i.e., first and second signaling moieties) semiconductor nanocrystals (i.e., ‘quantum dots’) whose absorption and emission spectra can be precisely controlled through the selection of nanoparticle material, size, and composition (see, e.g., Bruchez et al., Science 281:2013 (1998); Chan et al., J. Colloid and Interface Sci. 203:197 (1998), Han et al., Nature Biotechnol 19:631 (2001)).

The selection of a particular donor/acceptor pair is not critical to practicing the invention provided that energy can be transferred between the donor and the acceptor. P-(dimethyl aminophenylazo) benzoic acid (DABCYL) is one example of a non-fluorescent acceptor dye which effectively quenches fluorescence from an adjacent fluorophore, e.g., fluorescein or 5-(2′-aminoethyl) aminonaphthalene (EDANS).

The first and second signaling moieties can be attached to terminal or to non-terminal sequences. The position of the non-terminal sequences coupled to signaling moieties is limited to a maximal distance from the 5′ or 3′ nucleotide which still permits proximity dependent changes in the optical properties of the molecule. Coupling chemistries are routinely practiced in the art, and oligonucleotide synthesis services provided commercially (e.g., Integrated DNA Technologies, Coralville, Iowa) can also be used to generate labeled molecules. In a further embodiment, the nucleic acid sensor molecule is used, either tethered to a solid support or free in solution, to detect the presence and concentration of target molecules in a complex biological fluid.

For example, the first signaling moiety (F) can be fluorescein molecule coupled to the 5′ end and the second signaling molecule (D) can be a DABCYL molecule (a quenching group) coupled to the 3′ end. When the nucleic acid sensor molecule is not activated by target molecule, the fluorescent group and the quenching group are in close proximity and little fluorescence is detectable from the fluorescent group. Addition of target molecule causes a change in the conformation of the optical nucleic acid sensor molecule. When the molecule is activated by target recognition, and the first and second signaling moieties (F and D, respectively) are no longer in sufficient proximity for the quenching group to quench the fluorescence of the fluorescent group, the result is a detectable fluorescent signal being produced upon recognition of the target molecule.

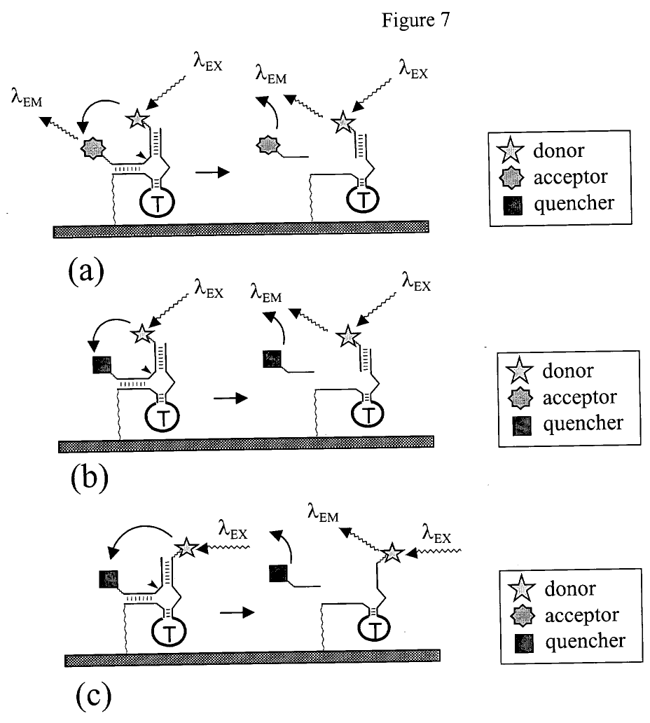

One general method for implementing a FRET-based (fluorescence resonance energy transfer) assay utilizing nucleic acid sensor molecules is described for a hammerhead nucleic acid sensor molecule, wherein the nucleic acid sensor molecule is immobilized on a solid substrate, e.g., within a microtiter plate well, on a membrane, on a glass or plastic microscope slide, etc. In the embodiment shown in FIGS. 7A, B, and C, a self-cleaving ribozyme such as the hammerhead (in this case attached to a solid support via a linker molecule is shown) is labeled with a fluorophore. In FIG. 7A, the labeled NASM in the unactivated state comprises two oligonucleotides including a transacting cleavage substrate which bears a first and second fluorescent label. In the unactivated state, i.e., in the absence of target molecule, the donor fluorophore and the acceptor fluorophore are in sufficiently close proximity for FRET to occur; thus, minimal fluorescent emission is detected from the donor fluorophore at wavelength 3, λ3, upon epi-illumination excitation at the excitation wavelength, λ_{EX}. Upon target molecule recognition, the cleavage fragment of the cleavage substrate bearing the acceptor fluorophore dissociates from the NASM-target complex. Once separated from the acceptor fluorophore, the donor fluorophore can no longer undergo de-excitation via FRET, resulting in a detectable increase in its fluorescent emission at wavelength, λ_{EM} (see, e.g., Singh. et al., RNA 5:1348 (1999); Wu et al., Anal. Biochem. 218:1 (1994); Walter et al., RNA 3:392 (1997); Walter et al., The EMBO Journal 17(8):2378 (1998)). In a further embodiment, the change in the polarization state of the fluorescent emission from the donor fluorophore (due to the increased diffusional rotation rate of the smaller cleavage fragment) can be detected/monitored in addition to changes in fluorescent emission intensity (see, e.g., Singh et al., Biotechniques 29:344 (2000)). In a further embodiment, the NASMs are free in solution.

In another embodiment, shown in FIG. 7B, the acceptor fluorophore attached to the cleavage substrate is replaced by a quencher group. This replacement will also result in minimal fluorescent donor emission at wavelength λ_{EX} when the NASM is in the unbound state under epi-illumination excitation at wavelength λ_{EX}. Upon target molecule recognition, the cleavage fragments of the cleavage substrate bearing the donor and quencher groups dissociate from the NASM-target molecule complex. Once separated from the quencher, the donor fluorophore will exhibit a detectable increase in its fluorescent emission at wavelength λ_{EM}. In a further embodiment, the change in the polarization state of the fluorescent emission from the donor fluorophore (due to the increased diffusional rotation rate of the smaller cleavage fragment) can be detected/monitored in addition to changes in fluorescent emission intensity. In a further embodiment, NASMs are free in solution.

In a different embodiment, the optical configuration is designed to provide excitation via total internal reflection (TIR)-illumination, as shown in FIG. 7C. Also, the donor fluorophore is attached to the NASM body while the quencher is attached to the cleavage substrate. In this configuration, with the surface-immobilized NASM in the unbound state, the fluorescent donor emission at wavelength λ_{EM} will be minimal. Upon target module recognition, the cleavage fragment of the cleavage substrate bearing the quencher group dissociates from the NASM-target module complex. Once separated from the quencher, the donor fluorophore will exhibit a detectable increase in its fluorescent emission at wavelength λ_{EM}. In an alternative embodiment to that shown in shown in FIG. 7C, the quencher group can be replaced with an acceptor fluorophore. In yet another alternative embodiment to those shown in FIGS. 7A, B, and C, the donor fluorophore is coupled to the cleavage fragment of the cleavage substrate and the acceptor fluorophore or quencher group is deleted. Upon target molecule recognition and dissociation of the cleavage fragment, the polarization state of the fluorescent emission from the donor fluorophore will undergo a detectable change due to the difference in the diffusional rotation rates of the surface-bound NASM target complex and the free cleavage fragment.

In one embodiment, a universal FRET trans-substrate is synthesized for all NASMs derived from self-cleaving allosteric ribozymes. This substrate would have complementary optical signaling units (i.e., donor and acceptor groups) coupled to opposite ends of the synthetic oligonucleotide sequence. Such a universal substrate would obviate the need for coupling optical signaling units to the sensor (i.e., ribozyme) molecule itself.

In addition to the herein described methods, any additional proximity dependent signaling system known in the art can be used to practice the method according to the invention, and are encompassed within the scope.

In one specific embodiment described here, a first oligonucleotide of the nucleotide sensor molecule is 3′-labeled with an acceptor or quencher fluorophore, such as TAMRA, AlexaFluor 568, or DABCYL, via specific periodate oxidation. A second oligonucleotide of the nucleic acid sensor molecule, complementary to at least part of the first oligo portion of the NASM, is labeled with a 3′ biotin and a 5′ donor fluorophore, such as fluorescein (FAM, FITC, etc.). These two nucleic oligonucleotides are heat-denatured in solution and allowed to anneal/hybridize during cooling to room temperature. After hybridization, the NASM solution is applied to a surface which has been coated with some type of avidin (streptavidin, neutravidin, avidin, etc.). This surface could include a microtiter plate well, a streptavidin-impregnated membrane, a glass or plastic microscope slide, etc. In any case, the ribozyme-oligo complex is specifically immobilized via the 3′ biotin on the donor oligo, leaving the binding domain free to interact with the target effector molecule.

The donor and acceptor fluorophores form an efficient FRET-pair; that is, upon excitation of the donor fluorophore near its spectral absorption maxima, the incident electromagnetic energy is efficiently transferred (nonradiatively) via resonant electric dipole coupling from the donor fluorophore to the acceptor fluorophore. The efficiency of this resonant energy transfer is strongly dependent on the separation between the donor and acceptor fluorophores, the transfer rate being proportional to 1R⁶, where R is the intermolecular separation. Therefore, when the donor and acceptor are in close proximity, i.e., a few bond-lengths or roughly 10-50 Angstroms, the fluorescent emission from donor species will be reduced relative to its output in an isolated configuration, while the emission from the acceptor species, through indirect excitation by the donor, will be detectable. Upon separation of the donor and acceptor, the donor fluorescence emission signal will increase strongly, while the acceptor emission signal will show a commensurate decrease in intensity. After effector-mediated cleavage at room temperature, the cleavage fragment will rapidly dissociate from the ribozyme body and diffuse away into solution.

This target-activated nucleic acid sensor molecule system constitutes a highly sensitive real-time sensor for detecting and quantitating the concentration of the target molecule present in an unknown sample solution. The ultimate limit of detection (LOD) for this system is determined by the switch factor, defined as the ratio of the catalytic rate (in this example, the rate of cleavage) of the ribozyme sensor in the presence of its target to that of the ribozyme in the absence of its target. The dynamic range of the ribozyme sensor will be determined by the switch factor and the dissociation constant, K_{d}, for the interaction of the ribozyme binding domain with the target molecule. In theory, the effective dynamic range over which the rate-response of the NASM is linear in the target concentration has K_{d} as an upper bound.

In practice, concentration measurements up to 1 mM are possible with this sensor in solution-phase measurements. The absolute precision of measurements made with this NASM will depend on the amount of background catalytic activity (i.e., in the absence of target) and baseline drift of the fluorescence signals from both sample and controls due to physical factors, such as liquid handling errors, reagent adhesion, evaporation, or mixing. After some optimization, run-to-run CVs of a few percent are possible with FRET-based NASMs measured in solution. Immobilization of the NASM does not degrade its catalytic activity, although it may limit the effective availability of the target-binding domain for interaction with target molecules. The locally high concentration of surface-immobilized NASM will tend to offset this effect by driving the equilibrium for the association (and subsequent catalytic) reactions toward formation of ribozyme-target complex. Detection of the fluorescent signals can be accomplished by a microplate fluorescence reader equipped with the appropriate lamps, optics, filters, and optical detectors (PMT) manufactured by Packard Instrument Co.

Such a sensor array could be used to detect and quantify the presence of an arbitrary target molecule in a complex solution, e.g., crude cell extract or biological fluid, in real time. In addition, this general NASM strategy could be extended to accomplish multiplexed detection of multiple analytes in a sample simultaneously, by using NASMs labeled with fluorophores having different emission wavelengths. In all of these scenarios, optical detection of the FRET signals could be accomplished using a commercially available microarray imager or scanning fluorescence microscope.

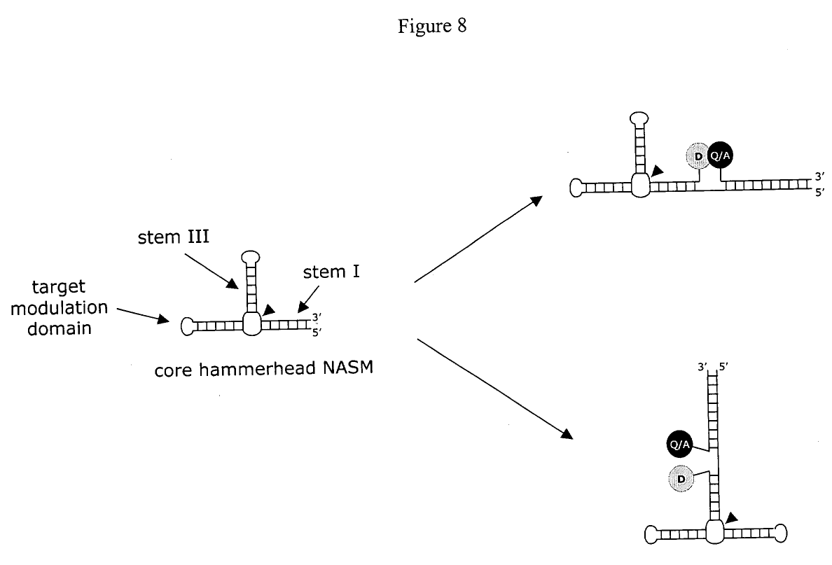

For example, fluorescence energy resonance transfer (FRET) can be used as a general detection method for hammerhead ribozyme or effector-dependent hammerhead ribozyme activity. Hammerhead NASMs typically consist of a catalytic domain responsible for RNA phoshodiester cleavage activity, plus a target modulation domain which, upon binding of an analyte molecule, triggers a structural change within the NASM and leads to the cleavage reaction. In one specific embodiment, described herein, such core hammerhead NASMs are modified to contain a donor fluorophore (D) covalently attached to the 3′-end of the NASM. In addition, a sequence domain to which a fluorescence quencher/acceptor dye (Q/A) containing auxiliary oligonucleotide can be hybridized is attached adjacent to either stem I or stem III (FIG. 8). The fluorophores are chosen to form an efficient FRET-pair; that is, upon excitation of the first, or donor fluorophore near its spectral absorption maxima, the incident electromagnetic energy is efficiently transferred (nonradiatively) via resonant electric dipole coupling from the donor fluorophore to the second, or acceptor fluorophore. The efficiency of this resonant energy transfer is strongly dependent on the separation between the donor and acceptor fluorophores, the transfer rate being proportional to 1/R⁶, where R is the intermolecular separation. Therefore, when the donor and acceptor are in close proximity, i.e., a few bond-lengths or roughly 10-50 Angstroms, the fluorescent emission from donor species will be reduced relative to its output in an isolated configuration, while the emission from the acceptor species, through indirect excitation by the donor, will be-detectable. Therefore the relative positioning of the fluorescence-labeled NASM 3′-terminus and the second fluorophore should be in close proximity to allow for such an energy transfer.

One example of FRET pairs are fluorescein as donor and TAMRA as acceptor. Alternatively, the acceptor can be replaced by a so-called dark quencher, such as DABCYL or QSY-7. Either relative orientation of the fluorophores (donor/acceptor and NASM/auxiliary oligo) can be chosen. The exact distance is governed by the number of unpaired nucleotides connecting stem I or III and the hybridization domain for the second oligo, and preferably is between 2 and 4 nucleotides long. The stem involving the 3′-terminus must be long enough to ensure proper folding into a hammerhead structure, but not too long to prevent rapid dissociation after hammerhead cleavage, and is preferably between 5 and 8 nucleotides. The attachment of the first fluorophore to the NASM 3′-terminus can be done by a variety of methods such as enzymatic ligation of a fluorescent nucleotide using terminal transferase or RNA ligase, or by oxidizing the terminal ribonucleotide with sodium periodate, followed by reaction with a fluorophore amine in the presence of sodium borohydride/cyanoborohydride, or a fluorophore hydrazide, semicarbazide or thiocarbazide (Agrawal in Protocols for Oligonucleotide Conjugates, Humana Press, Totowa, 1994, 26, 93; Wu et al., Nucleic Acids Research 24(17):3472 (1996)). Notably, apart from the 3′-modifications, the NASMs can be synthesized entirely through in simple vitro transcription reactions and do not have to contain any other internal or 5′ chemical modifications that are potentially difficult to introduce. The auxiliary oligonucleotide can be of any nucleotide sequence or composition (e.g., DNA, RNA, 2′-OMe-RNA, 2′-F—RNA or combination thereof), with a length ensuring tight hybridization to the complementary NASM domain, preferably between 20 to 30 nucleotides. Conversely the length and sequence of the corresponding NASM domain can be freely chosen to accommodate the auxiliary oligonucleotide.

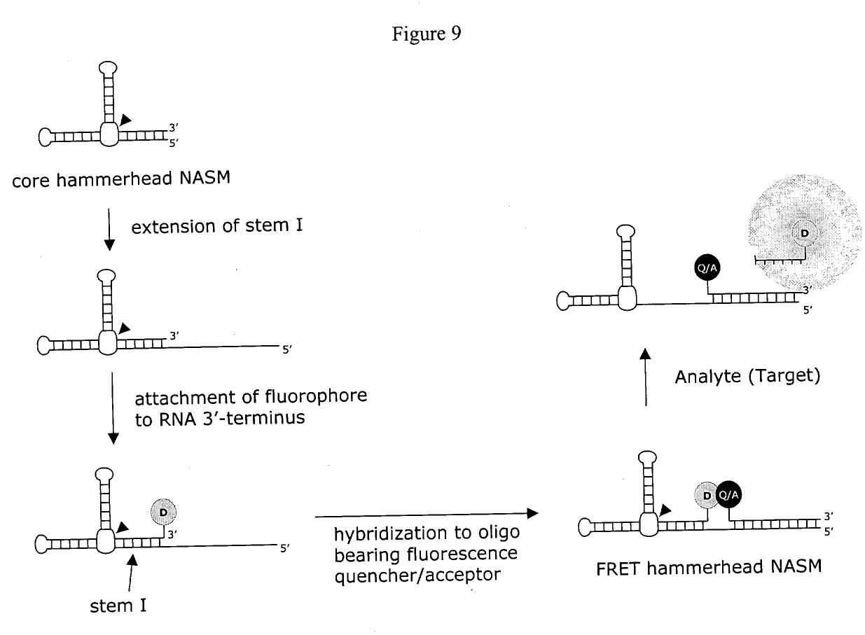

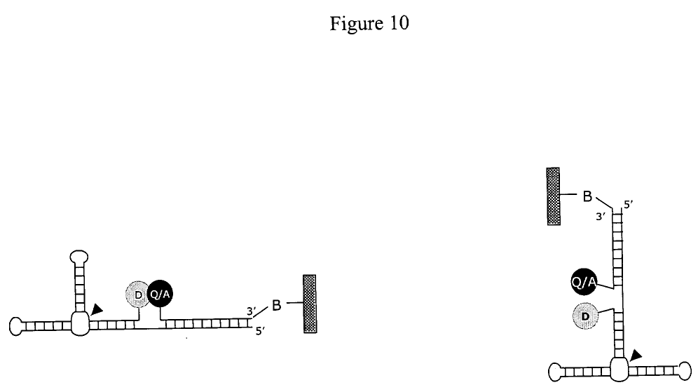

An example of a stem I-modified FRET hammerhead NASM is illustrated in FIG. 9. In addition, the NASM can be immobilized on a solid support via its auxiliary oligonucleotide, for example through incorporation of a biotin and capture on a streptavidin surface (FIG. 10). This surface could include a microfilter plate well, a streptavidin-impregnated membrane, a glass or plastic microscope slide, etc. Preferably immobilization takes place though the remote end of the auxiliary oligo, exposing the NASM core to the solution and not restricting it's accessibility or activity. The generalization of this application of surface-immobilized ribozyme sensors with FRET detection to a micro- or macro-arrayed format on an extended substrate such as glass or plastic is easily envisioned. Such a sensor array could be used to detect and quantify the presence of an arbitrary target molecule in a complex solution, e.g., crude cell extract or biological fluid, in real time. In this scenario, optical detection of the FRET signals could be accomplished using a commercially available microarray imager or scanning fluorescence microscope.

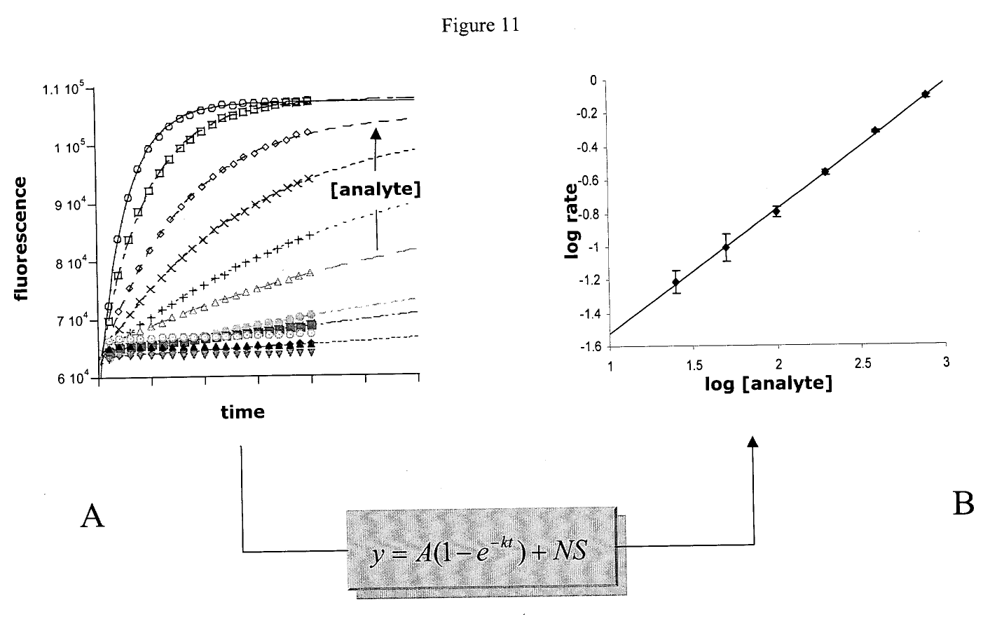

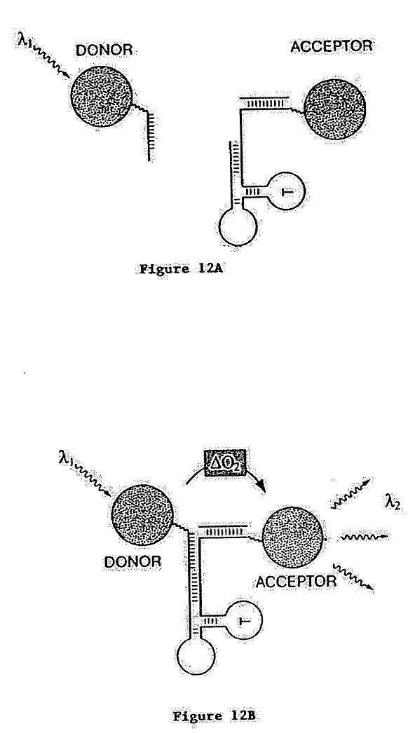

Upon effector-mediated cleavage of the hammerhead NASM, the 3′-terminus that contains one of the dye modifications is separated and dissociates away from the core NASM (FIG. 9). Thereby the donor and acceptor fluorophores are separated, leading to a strong increase in the donor fluorescence emission signal, while the acceptor emission signal will show a commensurate decrease in intensity. The increase or decrease in fluorescence can be recorded as a function of reaction time. Since the hammerhead NASM construct described herein exerts cis-cleavage activity, they follow a first-order cleavage kinetic model which allows the calculation of reaction rates after analysis of the resulting fluorescence vs. time curves (FIGS. 11A and 11B). Typically, within a certain range, the catalytic rate is a function of the effector concentration and can therefore be used to calculate an unknown effector concentration based on a measured rate value. This type of 1st order kinetic analysis in completely independent on the absolute fluorescent signal values, but relies only on their relative change over time. This makes this system particularly robust against signal fluctuations due to pipetting errors etc. compared to other, trans-reacting systems (i.e., hammerhead ribozymes acting on a separate substrate molecule).

To perform fluorescence resonance energy transfer (FRET) measurements, fluorescein-labeled RNA and quencher oligo are mixed to form the nucleic acid sensor cleavage solution. Cleavage reactions are performed in black 96-well microplates, and are started by mixing the nucleic acid sensor solution with target molecule in assay buffer. The fluorescence signals are monitored in a Fusion™ a-FP plate reader and the obtained fluorescence (rfu) values are plotted against time. The apparent reaction rates can be calculated assuming the 1st order kinetic model equation y=A(1−e^{-kt})+NS (A: signal amplitude; k: observed catalytic rate; NS: nonspecific background signal) using a curve fit algorithm (KaleidaGraph, Synergy Software, Reading, Pa.), as shown in FIG. 11. Dose-response curves are generated by plotting the calculated rates vs. the corresponding target concentrations.

ii) Indirect Energy Transfer

Other proximity-dependent signaling systems that do not rely on direct energy transfer between signaling moieties are also known in the art and can be used in the methods described herein. These include, e.g., systems in which a signaling moiety is stimulated to fluoresce or luminesce upon activation by the target molecule. This activation may be direct (e.g., as in the case of scintillation proximity assays (SPA), via a photon or radionucleide decay product emitted by the bound target), or indirect (e.g., as in the case of AlphaScreen™ assays, via reaction with singlet oxygen released from a photosensitized donor bead upon illumination). In both scenarios, the activation of detected signaling moiety is dependent on close proximity of the signaling moiety and the activating species. In general, for both fluorescence, fluorescence polarization, and scintillation-proximity-type assays, the nucleic acid sensor molecule may be utilized in either solution-phase or solid-phase formats. That is, in functional form, the nucleic acid sensor molecule may be tethered (directly, or via a linker) to a solid support or free in solution.

In one embodiment of an SPA assay, nucleic acid sensor molecules which ligate an oligonucleotide substrate in the presence of a target molecule, are bound to a scintillant-impregnated microwell plate (e.g., FlashPlates, NEN Life Sciences Products, Boston, Mass.) coated with, for example, streptavidin via a (biotin) linker attached to the 5′ end of a capture oligonucleotide sequence. The various plate-sensor coupling chemistries are determined by the type and manufacturer of the plates, and are well-known in the art. Upon the addition of a solution containing target molecule and excess radiolabeled (e.g., S) oligonucleotide substrate in ligation buffer, the NASMs hybridize and ligate the substrate oligonucleotide. Some fraction of the radiolabeled oligonucleotide substrate will be ligated to surface-immobilized NASMs on the plate, while unligated oligonucleotide substrate will be free in solution. Only those oligonucleotide substrates ligated to surface-immobilized NASMs on the plate will be in close enough proximity to the scintillant molecules embedded in the plate to excite them, thereby stimulating luminescence which can be easily detected using a luminometer (e.g., the TopCount luminescence plate reader, Packard Biosciences, Meriden, Conn.). This type of homogeneous assay format provides straightforward, real-time detection, quantification, and kinetic properties of target molecule binding.

In another embodiment, a similar SPA assay format is performed using scintillant-impregnated beads (e.g., Amersham Pharmacia Biotech, Inc., Piscataway, N.J.). In this embodiment, NASMs which ligate on an oligonucleotide substrate in the presence of a target molecule are coupled to scintillant-impregnated beads which are suspended in solution in, for example, a microwell plate. The various bead-sensor coupling chemistries are determined by the type and manufacturer of the beads, and are well-known in the art. Upon the addition of a solution containing target molecule and excess radiolabeled (e.g., S) oligonucleotide substrate in ligation buffer, the NASMs hybridize and ligate the oligonucleotide substrate. Some fraction of the radiolabeled substrate will be ligated to surface-immobilized NASMs on the beads, while unligated substrate will be free in solution. Only those substrates ligated to surface-immobilized NASMs on the beads will be in close enough proximity to the scintillant molecules embedded in the beads to excite them, thereby stimulating luminescence which can be easily detected using a luminometer (e.g., the TopCount luminescence plate reader, Packard Biosciences, Meriden, Conn.). In addition to enabling real-time target detection and quantification, this type of homogeneous assay format can be used to investigate cellular processes in situ in real time. This could be done by culturing cells directly onto a microwell plate and allowing uptake of scintillant beads and radioisotope by cells. Biosynthesis, proliferation, drug uptake, cell motility, etc. can then be monitored via the luminescence signal generated by beads in presence of selected target molecules (see, e.g., Cook et al., Pharmaceutical Manufacturing International pp. 49-53 (1992) or Heath et al., Cell Signaling: Experimental Strategies pp. 193-194 (1992)).

FIGS. 12A and 12B show an exemplary embodiment of a non-isotopic proximity assay based on nucleic acid sensor molecules used in conjunction with AlphaScreen™ beads (Packard Biosciences, Meriden, Conn.). In this embodiment, the nucleic acid sensor molecules, which ligate an oligonucleotide substrate in the presence of a target molecule, are bound to a chemiluminescent compound-impregnated acceptor bead coated with, for example, streptavidin, via a (biotin) linker attached to the 5′ end of the effector oligonucleotide sequence. The various bead-sensor coupling chemistries are determined by the type and manufacturer of the beads, and are well-known in the art. The oligonucleotide substrate is coupled to a photosensitizer-impregnated donor bead coated with, for example, streptavidin, via a (biotin) linker attached to the 3′ end of the substrate. The donor (substrate) and acceptor (ribozyme) beads and target molecules are then combined in solution in a microwell plate, some of the NASMs hybridize and ligate the oligonucleotide substrate, bringing the donor and acceptor beads into close proximity (<200 nm). Upon illumination at 680 nm, the photosensitizer in the donor bead converts ambient oxygen into the singlet state at a rate of approximately 60,000/second per bead. The singlet oxygen will diffuse a maximum distance of approximately 200 nm in solution; if an acceptor bead containing a chemiluminescent compound is within this range, i.e., if ligation has occurred in the presence of the target molecule, chemiluminescence at 370 nm is generated. This radiation is immediately converted within the acceptor bead to visible luminescence at 520-620 nm with a decay half-life of 0.3 sec. The visible luminescence at 520-620 nm is detected using a time-resolved fluorescence/luminescence plate reader (e.g., the Fusion multifunction plate reader, Packard Biosciences, Meriden, Conn.). This type of nonisotopic homogeneous proximity assay format provides highly sensitive detection and quantification of target molecule concentrations in volumes <25 microliters for high throughput screening (see, e.g., Beaudet et al., Genome Res. 11:600 (2001)). SPA assays can be performed with any type of NASM (i.e., endonucleases as well as ligases). This type of assay can also be used with the aptamers of the invention to monitor the presence or concentration of target in a solution.

iii) Optical Signal Generating Units with Single Signaling Moieties

In one embodiment, the optical nucleic acid sensor molecule comprises an optical signaling unit with a single signaling moiety introduced at either an internal or terminal position within the nucleic acid sensor molecule. In this embodiment, binding of the target molecule results in changes in both the conformation and physical aspect (e.g., molecular volume or mass, rotational diffusion rate, etc.) of the nucleic acid sensor molecule. Conformational changes in the nucleic acid sensor molecule upon target recognition will modify the chemical environment of the signaling moiety. Such a change in chemical environment will in general change the optical properties of the signaling moiety. Suitable signaling moieties are described in Jhaveri et al., Am. Chem. Soc. 122:2469-2473 (2000), and include, e.g., fluorescein, acridine, and other organic and nonorganic fluorophores.

In one embodiment, a signaling moiety is introduced at a position in the catalytic nucleic acid molecule near the target activation site (identifiable by footprinting studies, for example). Binding of the target molecule will (via a change in conformation of the nucleic acid molecule) alter the chemical environment and thus affect the optical properties of the signaling moiety in a detectable manner.

Recognition of the target molecule by the NASM will result in changes in the conformation and physical aspect of the nucleic acid sensor molecule, and will thus alter the kinetic properties of the signaling moiety. In particular, the changes in conformation and mass of the sensor-target complex will reduce the rotational diffusion rate for the sensor-target complex, resulting in a detectable change in the observed steady state fluorescence polarization (FP) from the signaling moiety. The expected change in FP signal with target concentration can be derived using a modified form of the well-known Michaelis-Menten model for ligand binding kinetics (see, e.g., Lakowicz, J. R., Principles of Fluorescence Spectroscopy, Second Edition, 1999, Kluwer Academic/Plenum Publishers, New York). FP is therefore a highly sensitive means of detecting and quantitatively determining the concentration of target molecules in a sample solution (Jameson et al., Methods in Enzymology 246:283 (1995); Jameson et al., METHODS 19:222 (1999); Jolley, Comb. Chem. High Throughput Screen 2(4):177 (1999); Singh, et al., BioTechniques 29:344 (2000); Owicki et al., Genetic Engineering News 17(19) (1997)). FP methods are capable of functioning in both solution- and solid-phase implementations.

Numerous additional methods can be used that, e.g., make use of a single fluorescent label and an unpaired guanosine residue (instead of a quencher group), to enable the use of FRET in target detection and quantitation as described in the embodiments above (see, e.g., Walter et al., RNA 3:392 (1997)).

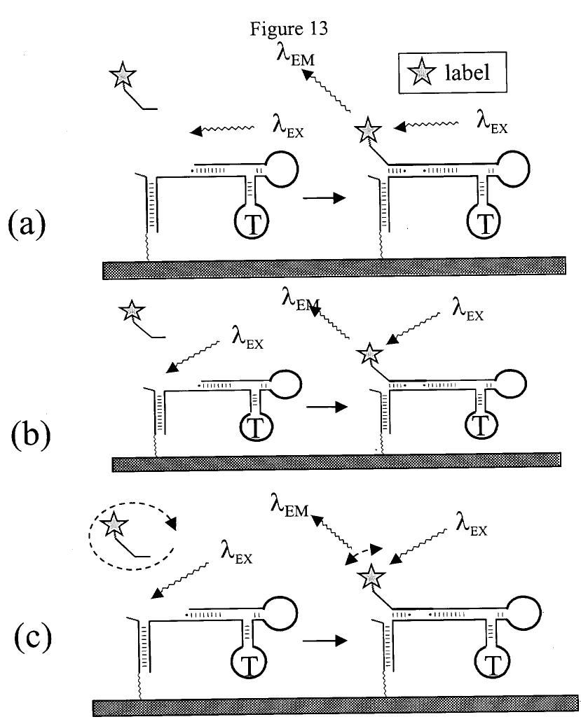

In a further embodiment, shown in FIG. 13A, B, and C, an unlabeled ligating NASM such as the lysozyme-dependent L1 ligase is shown (see, e.g., Robertson et al., Nucleic Acids Res. 28:1751-1759 (2000)). In the unactivated state, i.e., in the absence of target, no fluorescent emission is detected from the surface-bound NASMs under total internal reflection (TIR)-illumination (see FIG. 13A), or epi-illumination (see FIG. 13B). Upon recognition of target molecules in the presence of an oligonucleotide substrate with a tag (where the tag is capable of binding to a subsequently added fluorescent label via interactions including, but not limited to, biotin/streptavidin, amine/aldehyde, hydrazide, thiol, or other reactive groups) those oligonucleotide substrates hybridized to NASMs will undergo ligation and become covalently bonded to the thereto. In order to maximize the probability of hybridization for a given NASM, oligonucleotide substrate can be added in excess relative to NASM, the temperature of the ambient solution in which the reaction takes place can be kept below room temperature (e.g., 4° C.), and agitation of the reaction vessel can be employed to overcome the kinetic limitation of diffusion-limited transport of species in solution. Given the above conditions, as well as sufficient time for maximal hybridization and subsequent ligation to occur, fluorescent label with the appropriate reactive group to bind the substrate tag is added to the reaction mixture. Again, the degree of substrate-label binding can be maximized through control of label concentration, solution temperature, and agitation. Once the fluorescent label has bound to all available ligated substrate-NASM target complex, the solution temperature can be raised to drive off all of the hybridized but unligated substrate. With TIR-illumination, the spatial extent of the excitation region above the solid substrate surface to which the ribozymes are bound is only on the order of 100 nm. Therefore, the bulk solution above the substrate surface is not illuminated and the detected fluorescent emission will be primarily due to fluorophores which are bound to ligated oligonucleotide substrate-NASM-target molecule complexes tethered to the substrate surface. The fluorescence emission from surface-bound NASM-target molecule complexes in this homogeneous solid phase assay format represents an easily detectable optical signal. In another embodiment, the fluorescence polarization (FP) of the labeled substrate can be monitored, as shown in FIG. 13C. Upon ligation, the steady state fluorescence polarization signal from the substrate-NASM complex will increase detectably relative to the FP signal from the free labeled oligonucleotide substrate in solution, due to the difference in the diffusional rotation rates between the free and ligated forms.

In another embodiment, an unlabeled ligating NASM such as the lysozyme-dependent L1 ligase (see, e.g., Robertson et al., Nucleic Acids Res. 28:1751-1759 (2000)) is bound to a solid surface. In this embodiment, the oligonucleotide substrate is coupled to an enzyme-linked luminescent moiety, such as horseradish peroxidase (HRP) by a tag (where the tag is capable of binding to a subsequently added label via interactions including, but not limited to, biotin/streptavidin, amine/aldehyde, hydrazide, thiol, or other reactive groups). In the absence of target molecule, no luminescent emission is detected from the surface-bound NASMs. Upon recognition of target molecules in the presence of labeled oligonucleotide substrate, those oligonucleotide substrates hybridized to NASMs will undergo ligation and become covalently bonded to the NASMs. After removal of excess, unbound oligonucleotide substrate, the substrate for activation of the enzyme-linked luminescent label is added to the reaction volume. The resulting luminescent signal (e.g., from HRP, luciferase, etc.) is easily detectable using standard luminometers (e.g., the Fusion multifunction plate reader, Packard Bioscience). In a further embodiment, the activated solution can be precipitated, followed by colorimetric detection. In a particular embodiment, the enzyme linked signal amplification, TSA, (sometimes referred to as CARD-catalyzed reporter deposition) is an ultrasensitive detection method. The technology uses turnover of multiple tyramide substrates per horseradish peroxidase (HRP) enzyme to generate high-density labeling of a target protein or nucleic acid probe in situ. Tyramide signal amplification is a combination of three elementary processes: (1) Ligation (or not) of a biotinylated ligase oligonucleotide substrate oligo, followed by binding (or not) of a streptavidin-HRP to the probe; (2) HRP-mediated conversion of multiple copies of a fluorescent tyramide derivative to a highly reactive radical; and (3) Covalent binding of the reactive, short lived tyramide radicals to nearby nucleophilic residues, greatly reducing diffusion-related signal loss.

5) Generating Biosensors

Optical nucleic acid sensor molecules for the detection of a target molecule of interest are generated by first selecting catalytic nucleic acid molecules with catalytic activity modifiable (e.g., activatable) by a selected target molecule. In one embodiment, at least a portion of the catalytic site of the catalytic NASM is then removed and an optical signal generating unit is either added or inserted. Recognition of the target molecule by the nucleic acid sensor molecule activates a change in the properties of the optical signaling unit.

The nucleic acid sensor molecules can be, e.g., those which possess either ligating or cleaving activity in the presence of a target molecule.

One advantage of using nucleic acid sensor molecule arrays as opposed to protein arrays is the relative ease with which nucleic acid sensor molecules can be attached to chip surfaces. Immobilization of nucleic acid sensor molecules on a substrate provides a straightforward mechanism for carrying out multiple arrays in parallel. Initially, the optimal attachment chemistries are determined for use in immobilizing these molecules on a solid substrate. These molecules are further configured such that their activity and allosteric behavior is maintained following immobilization. Generally, the chip is configured such that it may be placed at the bottom of a sample holder and overlaid with sample solution, target and substrate oligonucleofide. Following an incubation to allow target present within the sample to activate catalysis, the sample is washed away and the extent of ribozyme catalysis quantified.

For example, endonuclease nucleic acid sensor molecules are generated by transcription in the presence of γ-thio-GTP (introducing a unique thiol at their 5′-end) and subsequently attached to a thiol-reactive surface (e.g. gold-coated polystyrene as described by Seetharaman et al., Nature Biotech 19:336 (2001)). Attachment methodologies are evaluated on the basis of the following criteria: efficiency, e.g., what is the yield of nucleic acid sensor molecule capture; capacity, e.g., what is the maximum concentration of nucleic acid sensor molecules that can be localized in a given spot size; stability, e.g., are ribozymes efficiently retained under a variety of solution conditions and during long-term storage; detection, e.g., do immobilization chemistries interfere with the ability to generate a detectable signal

To the extent that activity for immobilized nucleic acid sensor molecules is diminished, three different strategies for reconfiguring ribozymes for activity in solid phase applications are available: 1) immobilization chemistries, a variety of different immobilization chemistries are compared on the basis of their ability to maintain allosteric behavior. To the extent that they leave different surfaces available for protein effectors to interact with, that they tether different ends of the nucleic acid sensor molecules, and that they position the NASM either directly at the surface or displaced from the surface (in the case of streptavidin capture), different behaviors are observed depending upon the immobilization method. Protein-target activated NASMs have been shown to function in both direct and indirect attachment scenarios; 2) blocking chemistries, blocking agents (e.g., carrier proteins) are tested to determine whether losses in allosteric responsiveness are due to non-specific interactions between the allosteric activators and the chip surface; 3) tethers, steric effects may cause decreased catalytic activity upon direct end attachment to a solid support. Arbitrary sequence tethers are added as needed to increase the spacing between the attachment end and the core of the ribozyme.

Immobilized nucleic acid sensor molecules for target are prepared and are assayed for activity by monitoring either retention of end-labeled oligonucleotide substrate (for L1 ligase-based ribozymes) or release of end-labeled ribozyme (for endonucleases as originally described by Seetherman et al., Nature Biotech 19:336 (2001)). Radioactive tracers are used for labeling RNAs and substrates.

In one embodiment, a biosensor is provided which comprises a plurality of optical nucleic acid sensor molecules labeled with first and second signaling moieties specific for a target molecule. In another embodiment, the optical NASMs are labeled with a single signaling moiety. In one embodiment, the labeled nucleic acid sensor molecules are provided in a solution (e.g., a buffer). In another embodiment, the labeled nucleic acid sensor molecules are attached directly or indirectly (e.g., through a linker molecule) to a substrate. In further embodiments, nucleic acid sensor molecules can be synthesized directly onto the substrate. Suitable substrates which are encompassed within the scope include, e.g., glass or quartz, silicon, encapsulated or unencapsulated semiconductor nanocrystal materials (e.g., CdSe), nitrocellulose, nylon, plastic, and other polymers. Substrates may assume a variety of configurations (including, e.g., planar, slide shaped, wafers, chips, tubular, disc-like, beads, containers, or plates, such as microtiter plates, and other shapes).

Different chemistries for attaching nucleic acid sensor molecules to solid supports include: 1) conventional DNA arrays using aldehyde coated slides and 5′-amino modified oligonucleotides. The attached oligonucleotide serves as a capture tag that specifically hybridizes to a 3′-end extension on the ribozyme. Nucleic acid sensor molecule RNA treated with periodate to specifically introduce an aldehyde modification at the 3′-end. Modified RNA can be used either in a subsequent reaction with biotin hydrazide enables RNA capture on commercially-available streptavidin coated slides or in a subsequent reaction with adipic acid dihydrazide enables RNA capture on commercially-available aldehyde coated slides.

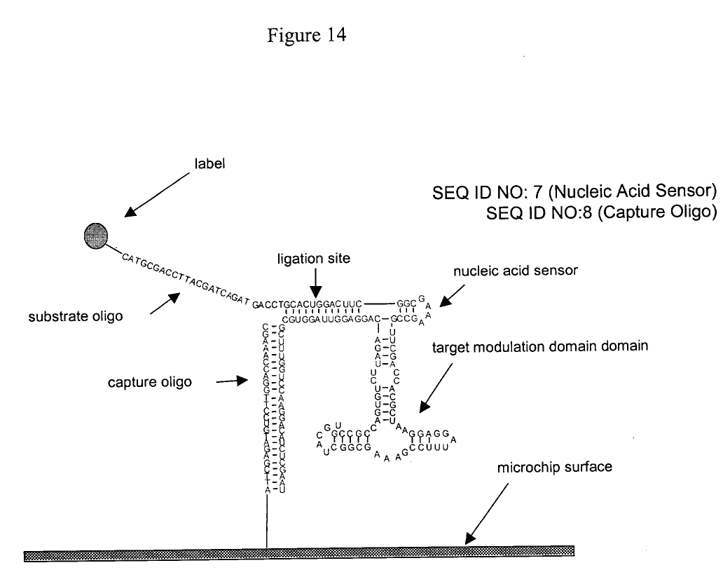

Numerous attachment chemistries, both direct and indirect, can be used to immobilize the sensor molecules on a solid support. These include, e.g., amine/aldehyde, biotin/streptavidin (avidin, neutravidin), ADH/oxidized 3′ RNA. In a particular embodiment, the nucleic acid sensor molecules ligate a substrate in the presence of a target molecule. In this embodiment the ribozymes are bound to a solid substrate via the effector oligonucleotide sequence as shown in FIG. 14.

In one embodiment, larger substrates can be generated by combining a plurality of smaller biosensors forming an array of biosensors. In a further embodiment, nucleic acid sensor molecules placed on the substrate are addressed (e.g., by specific linker or effector oligonucleotide sequences on the nucleic acid sensor molecule) and information relating to the location of each nucleic acid sensor molecule and its target molecule specificity is stored within a processor. This technique is known as spatial addressing or spatial multiplexing. Techniques for addressing nucleic acids on substrates are known in the art and are described in, for example, U.S. Pat. No. 6,060,252; U.S. Pat. No. 6,051,380; U.S. Pat. No. 5,763,263; U.S. Pat. No. 5,763,175; and U.S. Pat. No. 5,741,462.

In another embodiment, a manual or computer-controlled robotic microarrayer is used to generate arrays of nucleic acid sensor molecules immobilized on a solid substrate. In one embodiment, the arrayer utilizes contact-printing technology (i.e., it utilizes printing pins of metal, glass, etc., with or without quill-slots or other modifications). In a different embodiment, the arrayer utilizes non-contact printing technology (i.e., it utilizes ink jet or capillary-based technologies, or other means of dispensing a solution containing the material to be arrayed). Numerous methods for preparing, processing, and analyzing microarrays are known in the art (see Schena et al., Microarray Biochip Technology, ed. pp. 1-18 (2000); Mace et al., Microarray Biochip Technology, ed. pp. 39-64 (2000); Heller et al., Academic Press, Inc. pp. 245-256 (1999); Basararsky et al., Microarray Biochip Technology, ed. pp. 265-284 (2000); Schermer, DNA Microarrays a Practical Approach pp. 17-42 (1999)). Robotic and manual arrayers are commercially available including, for example, the SpotArray from Packard Biosciences, Meriden, Conn., and the RA-1 from GenomicSolutions, Ann Arbor, Mich.

In another embodiment, different nucleic acid sensor molecules are immobilized on a streptavidin-derivatized substrate via biotin linkers. The individual sensor spots can be manually arrayed. For example, NASM can hybridize to a biotin-linked capture oligo, which in turn will bind to a streptavidin coated surface.

Solution measurements of target molecule concentration can be made by bathing the surface of the biosensor array in a solution containing the targets (analytes) of interest. In practice this is accomplished either by incorporating the array within a microflowcell (with a flow rate of ˜25 microliters/min), or by placing a small volume (˜6-10 microliters) of the target solution on the array surface and covering it with a cover slip. Detection and quantification of target concentration is accomplished by monitoring changes in the fluorescence polarization (FP) signal emitted from the fluorescein label under illumination by 488 nm laser radiation. The rotational diffusion rate is inversely proportional to the molecular volume; thus the rotational correlation time for the roughly 20-nucleotide unbound sensor (i.e., in the absence of target molecule) will be significantly less than that for the target-NASM complex. The fluorescence emission from the target-NASM complex will therefore experience greater residual polarization due to the smaller angle through which the emission dipole axis of the sensor fluorophore can rotate within its radiative lifetime. In another embodiment, different surface attachment chemistries are used to immobilize the NASMs on a solid substrate. As previously noted, these include, e.g., interactions involving biotin/streptavidin, amine/aldehyde, hydrazide, thiol, or other reactive groups.

One type of array includes immobilized effector oligonucleotides with terminal amine groups attached to a solid substrate derivatized with aldehyde groups. This array can be used to spatially address (i.e., the sequence of nucleotides for each effector oligonucleotide can be synthesized as a cognate to the effector oligonucleotide binding domain of a nucleic acid sensor molecule specific for a particular target molecule) and immobilize the nucleic acid sensor molecules prior to their use in a solid-phase assay (see, e.g., Zammatteo et al., Anal Biochem 280:143 (2000)).

For example, to attach effector oligonucleotides to aldehyde derivatized substrate, discrete spots of solution containing effector oligonucleotides with amine-reactive terminal groups or linkers with terminal amine groups using microarraying pins, pipette, etc are printed and then allowed to dry to dry for 12 hrs. at room temperature and <30% relative humidity. The substrate is then rinsed twice with dH₂O containing 0.2% SDS for 2 min. with vigorous agitation at room temperature. The substrate is then rinsed once in dH₂O for 2 min. with vigorous agitation at room temperature and transferred to boiling (100° C.) dH₂O for 3 min. to denature DNA. The denatured substrate is then dried by centrifuging at 500×g for 1 min. and then treated with 0.1 M NaBH₄ in phosphate buffered saline (PBS, pH 7) for 5 min. with mild agitation at room temperature. Following NaBH₄ treatment, the substrate is rinsed twice in dH₂O containing 0.2% SDS for 1 min. with vigorous agitation at room temperature and then washed once with dH₂O for 2 min. with vigorous agitation at room temperature. The substrate is again boiled in dH₂O (100° C.) for 10 sec. to denature DNA. The substrate is dried by centrifugation as described above and stored at 4° C. prior to hybridization.

In the case where it is desirable to immobilize an array of NASMs by direct attachment to a solid surface, the nucleic acid sensor molecules are bound to a solid substrate directly via their 3′ termini. The attachment is accomplished by oxidation (using, e.g., sodium periodate) of the 3′ vicinal diol of the nucleic acid sensor molecule to an aldehyde group. This aldehyde group will react with a hydrazide group to form a hydrazone bond. The hydrazone bond is quite stable to hydrolysis, etc., but can be further reduced (for example, by treatment with NaBH₄ or NaCNBH₃). The use of adipic acid dihydrazide (ADH, a bifunctional linker) to derivatize an aldehyde surface results in a hydrazide-derivatized surface which provides a linker of approximately 10 atoms between the substrate surface and point of biomolecular attachment (see Ruhn et al., J. Chromatography A 669:9. (1994); O'Shaughnessy, J. Chromatography 510:13 (1990); Roberston et al., Biochemistry 11 (4):533 (1972); Schluep et al., Bioseparation 7:317 (1999); Chan et al., J. Colloid and Interface Sci. 203:197 (1998)).

A hydrazide-terminated surface can be prepared by ADH treatment of the aldehyde substrate. Briefly, to 50 mL of 0.1 M phosphate buffer (pH 5) 100-fold excess of adipic acid dihydrazide (ADH) relative to concentration of aldehyde groups is added on substrate surface. The substrate is then placed in a 50 mL tube containing the ADH in phosphate buffer and shaken mixture for 2 h. Following incubation, the substrate is washed 4-times with 0.1 M phosphate buffer (pH 7). The free aldehyde groups on the substrate surface are then reduced by treatment with a 25-fold excess of NaBH₄ or NaCNBH₃ in 0.1 M phosphate buffer in a 50 ml conical tube with shaking for 90 min. The substrate is then washed 4-times with 0.1 M phosphate buffer (pH 7) and stored 0.1 M phosphate buffer (pH 7) at 4° C. until use.

Nucleic acid molecules for specific coupling to the ADH-terminated surface via their 3′ termini are prepared by periodate oxidation of the RNA, see, e.g., Proudnikov et al., Nucleic Acid Res. 24(22):4535 (1996); Wu et al., Nucleic Acids Res. 24(17):3472 (1996). Briefly, up to 20 μg RNA in 5 μl of H₂O at 20° C. is treated with 1 ml 0.1 M NaIO₄ (˜20-fold excess relative to RNA). The RNA is incubated with the NaIO₄ for 30 min. in a light-tight tube prior to the addition of 1 ml 0.2 M Na sulphite (˜2-fold excess relative to NaIO₄) to stop the reaction (30 min.; room temperature). The oxidized RNA is then recovered by ethanol precipitation and a spin-separation column.

The specificity of the biosensors and NASMs according to the invention is determined by the specificity of the target modulation domain of the nucleic acid sensor molecule. In one embodiment, a biosensor is provided in which all of the nucleic acid sensor molecules recognize the same molecule. In another embodiment, a biosensor is provided which can recognize at least two different target molecules allowing for multi-analyte detection. Multiple analytes can be distinguished by using different combinations of first and second signaling molecules. In addition to the wavelength/color and spatial multiplexing techniques previously described, biosensors may be used to detect multiple analytes using intensity multiplexing. This is accomplished by varying the number of fluorescent label molecules on each biosensor in a controlled fashion. Since a single fluorescent label is the smallest integral labeling unit possible, the number of fluorophores (i.e., the intensity from) a given biosensor molecule provides a multiplexing index. Using the combination of 6-wavelength (color) and 10-level intensity multiplexing, implemented in the context of semiconductor nanocrystals derivatized as bioconjugates, would theoretically allow the encoding of million different analyte-specific biosensors (Han et al., Nature Biotechnol. 19:631 (2001)).

In one embodiment, multiple single target biosensors can be combined to form a multianalyte detection system which is either solution-based or substrate-based according to the needs of the user. In this embodiment, individual biosensors can be later removed from the system, if the user desires to return to a single analyte detection system (e.g., using target molecules bound to supports, or, for example, manually removing a selected biosensor(s) in the case of substrate-based biosensors). In a further embodiment, nucleic acid sensor molecules binding to multiple analytes are distinguished from each other by referring to the address of the nucleic acid sensor molecule on a substrate and correlating its location with the appropriate target molecule to which it binds (previously described as spatial addressing or multiplexing).

In one embodiment, subsections of a biosensor array can be individually subjected to separate analyte solutions by use of substrate partitions or enclosures that prevent fluid flow between subarrays, and microfluidic pathways and injectors to introduce the different analyte solutions to the appropriate sensor subarray.

6) Nucleic Acid Sensor Molecule and Biosensor Systems

In one embodiment, a nucleic acid sensor molecule or biosensor system is provided comprising a nucleic acid sensor molecule in communication with a detector system. In a further embodiment, a processor is provided to process optical signals detected by the detector system. In still a further embodiment, the processor is connectable to a server which is also connectable to other processors. In this embodiment, optical data obtained at a site where the NASM or biosensor system resides can be transmitted through the server and data is obtained, and a report displayed on the display of the off-site processor within seconds of the transmission of the optical data. In one embodiment, data from patients is stored in a database which can be accessed by a user of the system.

Data obtainable from the biosensors according to the invention include diagnostic data, data relating to lead compound development, and nucleic acid sensor molecule modeling data (e.g., information correlating the sequence of individual sensor molecules with specificity for a particular target molecule). In one embodiment, these data are stored in a computer database. In a further embodiment, the database includes, along with diagnostic data obtained from a sample by the biosensor, information relating to a particular patient, such as medical history and billing information. Although, in one embodiment, the database is part of the nucleic acid sensor molecule system, the database can be used separately with other detection assay methods and drug development methods.

Detectors used with the nucleic acid sensor molecule systems according to the invention, can vary, and include any suitable detectors for detecting optical changes in nucleic acid molecules. These include, e.g., photomultiplier tubes (PMTs), charge coupled devices (CCDs), intensified CCDs, and avalanche photodiodes (APDs). In one embodiment, an optical nucleic acid sensor molecule is excited by a light source in communication with the biosensor. In a further embodiment, when the optical signaling unit comprises first and second signal moieties that are donor/acceptor pairs (i.e., signal generation relies on the fluorescence of a donor molecule when it is removed from the proximity of a quencher acceptor molecule), recognition of a target molecule will cause a large increase in fluorescence emission intensity over a low background signal level. The high signal-to-noise ratio permits small signals to be measured using high-gain detectors, such as PMTs or APDs. Using intensified CCDs, and PMTs, single molecule fluorescence measurements have been made by monitoring the fluorescence emission, and changes in fluorescence lifetime, from donor/acceptor FRET pairs (see, e.g., Sako, et al., Nature Cell Bio. 2:168 (2000); Lakowicz et al, Rev. Sci. Instr. 62(7):1727 (1991)).

Light sources include, e.g., filtered, wide-spectrum light sources, (e.g., tungsten, or xenon arc), laser light sources, such as gas lasers, solid state crystal lasers, semiconductor diode lasers (including multiple quantum well, distributed feedback, and vertical cavity surface emitting lasers (VCSELs)), dye lasers, metallic vapor lasers, free electron lasers, and lasers using any other substance as a gain medium. Common gas lasers include Argon-ion, Krypton-ion, and mixed gas (e.g., Ar—Kr) ion lasers, emitting at 455, 458, 466, 476, 488, 496, 502, 514, and 528 nm (Ar ion); and 406, 413, 415, 468, 476, 482, 520, 531, 568, 647, and 676 nm (Kr ion). Also included in gas lasers are Helium Neon lasers emitting at 543, 594, 612, and 633 nm. Typical output lines from solid state crystal lasers include 532 nm (doubled Nd:YAG) and 408/816 nm (doubled/primary from Ti: Sapphire). Typical output lines from semiconductor diode lasers are 635, 650, 670, and 780 nm.

Excitation wavelengths and emission detection wavelengths will vary depending on the signaling moieties used. In one embodiment, where the first and second signaling moieties are fluorescein and DABCYL, the excitation wavelength is 488 nm and the emission wavelength is 514 nm. In the case of semiconductor nanocrystal-based fluorescent labels, a single excitation wavelength or broadband UV source may be used to excite several probes with widely spectrally separated emission wavelengths (see Bruchez et al., Science 281:2013 (1998); Chan et al., J. Colloid and Interface Sci. 203:197 (1998)).

In one embodiment, detection of changes in the optical properties of the nucleic acid sensor molecules is performed using any of a cooled CCD camera, a cooled intensified CCD camera, a single-photon-counting detector (e.g., PMT or APD), or other light sensitive sensor. In one embodiment, the detector is optically coupled to the nucleic acid sensor molecule through a lens system, such as in an optical microscope (e.g., a confocal microscope). In another embodiment, a fiber optic coupler is used, where the input to the optical fiber is placed in close proximity to the substrate surface of a biosensor, either above or below the substrate. In yet another embodiment, the optical fiber provides the substrate for the attachment of nucleic acid sensor molecules and the biosensor is an integral part of the optical fiber.

In one embodiment, the interior surface of a glass or plastic capillary tube provides the substrate for the attachment of nucleic acid sensor molecules. The capillary can be either circular or rectangular in cross-section, and of any dimension. The capillary section containing the biosensors can be integrated into a microfluidic liquid-handling system which can inject different wash, buffer, and analyte-containing solutions through the sensor tube. Spatial encoding of the sensors can be accomplished by patterning them longitudinally along the axis of the tube, as well as radially, around the circumference of the tube interior. Excitation can be accomplished by coupling a laser source (e.g., using a shaped output beam, such as from a VCSEL) into the glass or plastic layer forming the capillary tube. The coupled excitation light will undergo TIR at the interior surface/solution interface of the tube, thus selectively exciting fluorescently labeled biosensors attached to the tube walls, but not the bulk solution. In one embodiment, detection can be accomplished using a lens-coupled or proximity-coupled large area segmented (pixelated) detector, such as a CCD. In a particular embodiment, a scanning (i.e., longitudinal/axial and azimuthal) microscope objective lens/emission filter combination is used to image the biosensor substrate onto a CCD detector. In a different embodiment, a high resolution CCD detector with an emission filter in front of it is placed in extremely close proximity to the capillary to allow direct imaging of the biosensors. In a different embodiment, highly efficient detection is accomplished using a mirrored tubular cavity that is elliptical in cross-section. The sensor tube is placed along one focal axis of the cavity, while a side-window PMT is placed along the other focal axis with an emission filter in front of it. Any light emitted from the biosensor tube in any direction will be collected by the cavity and focused onto the window of the PMT.

In still another embodiment, the optical properties of a nucleic acid sensor molecule are analyzed using a spectrometer (e.g., such as a luminescence spectrometer) which is in communication with the biosensor. The spectrometer can perform wavelength discrimination for excitation and detection using either monochromators (i.e., diffraction gratings), or wavelength bandpass filters. In this embodiment, biosensor molecules are excited at absorption maxima appropriate to the signal labeling moieties being used (e.g., acridine at 450 nm, fluorescein at 495 nm) and fluorescence intensity is measured at emission wavelengths appropriate for the labeling moiety used (e.g., acridine at 495 mm; fluorescein at 515 nm). Achieving sufficient spectral separation (i.e., a large enough Stokes shift) between the excitation wavelength and the emission wavelength is critical to the ultimate limit of detection sensitivity. Given that the intensity of the excitation light is much greater than that of the emitted fluorescence, even a small fraction of the excitation light being detected or amplified by the detection system will obscure a weak biosensor fluorescence emission signal. In one embodiment, the biosensor molecules are in solution and are pipetted (either manually or robotically) into a cuvette or a well in a microtiter plate within the spectrometer. In a further embodiment, the spectrometer is a multifunction plate reader capable of detecting optical changes in fluorescence or luminescence intensity (at one or more wavelengths), time-resolved fluorescence, fluorescence polarization (FP), absorbance (epi and transmitted), etc., such as the Fusion multifunction plate reader system (Packard Biosciences, Meriden, Conn.). Such a system can be used to detect optical changes in biosensors either in solution, bound to the surface of microwells in plates, or immobilized on the surface of solid substrate (e.g., a biosensor microarray on a glass substrate). This type of multiplate/multisubstrate detection system, coupled with robotic liquid handling and sample manipulation, is particularly amenable to high-throughput, low-volume assay formats.

In embodiments where nucleic acid sensor molecules are attached to substrates, such as a glass slide or in microarray format, it is desirable to reject any stray or background light in order to permit the detection of very low intensity fluorescence signals. In one embodiment, a small sample volume (˜10 nL) is probed to obtain spatial discrimination by using an appropriate optical configuration, such as evanescent excitation or confocal imaging. Furthermore, background light can be minimized by the use of narrow-bandpass wavelength filters between the sample and the detector and by using opaque shielding to remove any ambient light from the measurement system.

In one embodiment, spatial discrimination of nucleic acid sensor molecules attached to a substrate in a direction normal to the interface of the substrate (i.e., excitation of only a small thickness of the solution layer directly above and surrounding the plane of attachment of the biosensor molecules to the substrate surface) is obtained by evanescent wave excitation. Evanescent wave excitation utilizes electromagnetic energy that propagates into the lower-index of refraction medium when an electromagnetic wave is totally internally reflected at the interface between higher and lower-refractive index materials. In this embodiment a collimated laser beam is incident on the substrate/solution interface (at which the biosensors are immobilized) at an angle greater than the critical angle for total internal reflection (TIR). This can be accomplished by directing light into a suitably shaped prism or an optical fiber. In the case of a prism, the substrate is optically coupled (via index-matching fluid) to the upper surface of the prism, such that TIR occurs at the substrate/solution interface on which the biosensors are immobilized. Using this method, excitation can be localized to within a few hundred nanometers of the substrate/solution interface, thus eliminating autofluorescence background from the bulk analyte solution, optics, or substrate. Target recognition is detected by a change in the fluorescent emission of the nucleic acid sensor, whether a change in intensity or polarization. Spatial discrimination in the plane of the interface (i.e., laterally) is achieved by the optical system.

In one embodiment, a large area of the biosensor substrate is uniformly illuminated, either via evanescent wave excitation or epi-illumination from above, and the detected signal is spatially encoded through the use of a pixelated detector, such as CCD camera. An example of this type of uniform illumination/CCD detection system (using epi-illumination) for the case of microarrayed biosensors on solid substrates is the GeneTAC 2000 scanner (GenomicSolutions, Ann Arbor, Mich.). In a different embodiment, a small area (e.g., 10×10 microns to 100×100 microns) of the biosensor substrate is illuminated by a micro-collimated beam or focused spot. In one embodiment, the excitation spot is rastered in a 2-dimensional scan across the static biosensor substrate surface and the signal detected (with an integrating detector, such as a PMT) at each point correlated with the spatial location of that point on the biosensor substrate (e.g., by the mechanical positioning system responsible for scanning the excitation spot). Two examples of this type of moving spot detection system for the case of microarrayed biosensors on solid substrates are: the DNAScope scanner (confocal, epi-illumination, GeneFocus, Waterloo, ON, Canada), and the LS IV scanner (non-confocal, epi-illumination, GenomicSolutions, Ann Arbor, Mich.). In yet another embodiment, a small area (e.g., 10×10 microns to 100×100 microns) of the biosensor substrate is illuminated by a stationary micro-collimated beam or focused spot, and the biosensor substrate is rastered in a 2-dimensional scan beneath the static excitation spot, with the signal detected (with an integrating detector, such as a PMT) at each point correlated with the spatial location of that point on the biosensor substrate (e.g., by the mechanical positioning system responsible for scanning the substrate). An example of this type of moving substrate detection (using confocal epi-illumination) system for the case of microarrayed biosensors on solid substrates is the ScanArray 5000 scanner (Packard Biochip, Billerica, Mass.).

For example, a TIR evanescent wave excitation optical configuration is implemented, with a static substrate and dual-capability detection system. The detection system is built on the frame of a Zeiss universal fluorescence microscope. The system is equipped with 2 PMTs on one optical port, and an intensified CCD camera (Cooke, St. Louis, Mo.) mounted on the other optical port. The optical path utilizes a moveable mirror which can direct the collimated, polarized laser beam through focusing optics to form a spot, or a beam expander to form a large (>1 cm) beam whose central portion is roughly uniform over the field of view of the objective lens. Another movable mirror can direct the light either to the intensified CCD camera when using large area uniform illumination, or to the PMTs in the scanned spot mode. In spot scanning mode, a polarizing beamsplitter separates the parallel and perpendicular components of the emitted fluorescence and directs each to its designated PMT. An emission filter in the optical column rejects scattered excitation light from either type of detector. In CCD imaging mode, manually adjusted polarizers in the optical column of the microscope must be adjusted to obtain parallel and perpendicular images from which the fluorescence polarization or anisotropy can be calculated. A software program interfaces with data acquisition boards in a computer which acquires the digital output data from both PMTs and CCD. This program also controls the PMT power, electromechanical shutters, and galvanometer mirror scanner, calculates and plots fluorescence polarization in real time, and displays FP and intensity images.

In another embodiment, the detection system is a single photon counter system (see, e.g., U.S. Pat. No. 6,016,195 and U.S. Pat. No. 5,866,348) requiring rastering of the sensor substrate to image larger areas and survey the different binding regions on the biosensor.

In another embodiment of the invention, the biosensor is used to detect a target molecule through changes in the electrochemical properties of the nucleic acid sensor molecules in close proximity to it which occur upon recognition of the target by the NASM.

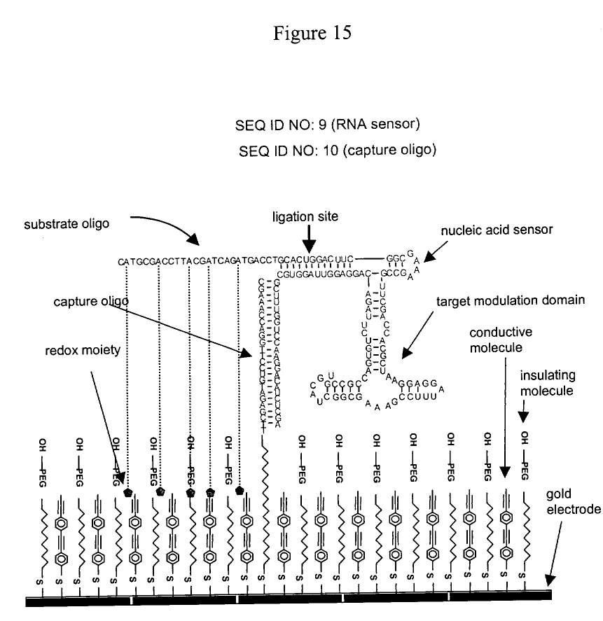

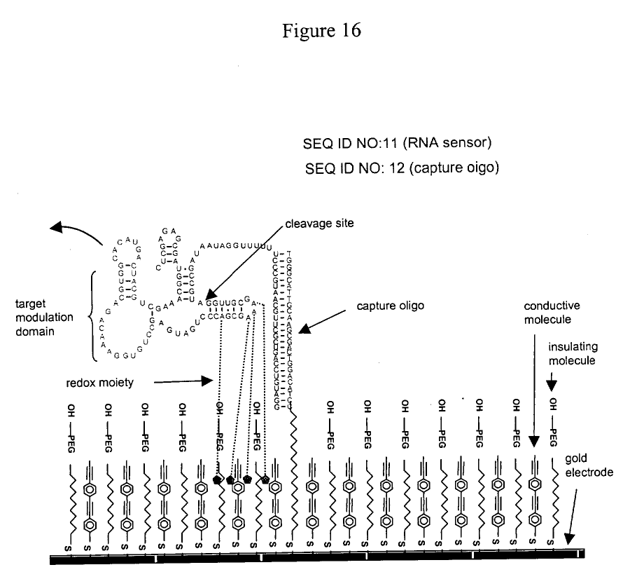

In a one embodiment, the biosensor system consists of three major components: 1) optical nucleic acid sensor molecules immobilized on an array of independently addressable gold electrodes. The nucleic acid sensor molecules immobilized on each electrode may be modulated by the same or different target molecules, including proteins, metabolites and other small molecules, etc.; 2) an oligonucleotide substrate which acts as a signaling probe, hybridizing to the oligonucleotide substrate binding domain of the ligase sensor and forming a covalent phosphodiester bond with the nucleic acid sensor molecule nucleotide adjacent to its 3′ terminus in the presence of the appropriate target. This oligonucleotide substrate is typically a nucleic acid sequence containing one or more modified nucleotides conjugated to redox active metallic complexes, e.g., ferrocene moieties, which can act as electron donors; and 3) an immobilized mixed self-assembled surface monolayer (SAM), comprised of conductive species separated by insulating species, covering the surface of the electrodes, as shown in FIGS. 15 and 16. Examples of conductive species include thiol-terminated linear molecules, such as oligophenylethyl molecules, while examples of nonconductive thiol-terminated linear molecules, include alkane-thiol molecules terminated with polyethylene glycol (PEG). All immobilized species can be covalently attached to the electrode surface by terminal thiol groups. Upon recognition of the target molecule by the target modulation domain and subsequent ligation of the oligonucleotide substrate, the redox active signaling moieties coupled to the substrate oligo will be brought into close proximity to the conductive surface layer, resulting in a detectable increase in electronic surface signal.

In another preferred embodiment, the biosensor system consists of two major components: (1) Optical nucleic acid sensor molecules immobilized on an array of independent addressable gold electrodes. The nucleic acid sensor molecules immobilized on each electrode may be modulated by the same or different target molecules, including proteins, metabolites and other small molecules, etc. The NASM will contain one or more nucleotides conjugated to redox active metallic complexes, e.g., ferrocene moieties, which can act as electron donors; and (2) an immobilized mixed self-assembled surface monolayer (SAM), comprised of conductive species separated by insulating species, covering the surface of the electrodes. Examples of conductive species include thiol-terminated linear molecules, such as oligophenylethyl molecules, while examples of nonconductive thiol-terminated linear molecules include alkane-thiol molecules terminated with polyethylene glycol (PEG). The SAM-coated molecule can be immobilized via a capture oligonucleotide. In this case, the redox active signaling moieties are coupled to the body of the NASM. Upon recognition of the target molecule by the target modulation domain and subsequent cleavage, the bulk of the NASM, including the nucleotides coupled to the redox active signaling moieties, will dissociate from the surface, resulting in a detectable loss of electronic current signal.

In another embodiment, the array would be subjected, e.g., by an integrated microfluidic flowcell, to an analyte solution containing the target(s) of interest at some unknown concentration. The range of possible sample analyte solutions may include standard buffers, biological fluids, and cell or tissue extracts. The sample solution will also contain the signaling probe at a saturating concentration relative to the immobilized nucleic acid sensor molecule. This ensures that at any given time during analysis, there is a high probability that each nucleic acid sensor molecule will have a signaling probe hybridized to it. In the presence of the target molecules in the sample solution, the nucleic acid sensor molecule will form a covalent phosphodiester bond, i.e., ligate, with the signaling probe, thus immobilizing it with its redox active electron donor species in electrical contact with the conductive molecules within the mixed self-assembled surface monolayer. After some integration time, during which signal probe ligation occurs, it may be necessary to denature the hybridized but unligated signaling probes. This denaturation step, which effectively removes ‘background’ signaling probes and their associated redox moieties from the vicinity of the electrodes can be accomplished by a small temperature increase (e.g., from 21° C. to 25° C.), or by a brief negative voltage spike applied to the sensor electrodes followed by the application of a large positive DC voltage to a separate electrode that would collect unligated signaling. For the case of a sufficiently short hybridization region, e.g., 5 base-pairs, on the signaling probe, a separate denaturation step may not be necessary. In either case, following nucleic acid sensor molecule activation by target molecules, a linear electrical potential ramp is applied to the electrodes. The redox species conjugated to the immobilized signaling probe-nucleic acid sensor molecule will be electrochemically oxidized, liberating one or more electrons per moiety. The conductive molecules within the surface monolayer will provide an electrical path for the liberated electrons to the electrode surface.

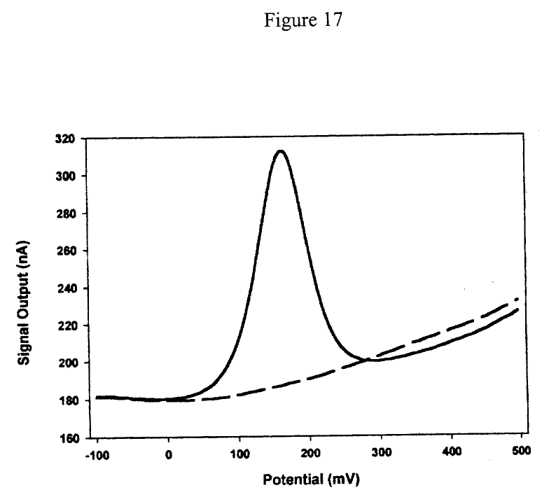

The net electron transfer to or from the electrode will be measured as a peak in the faradaic current, centered at the redox potential of the electron donor species (specified for a given reference electrode) and superposed on top of the capacitive current baseline which is observed in the absence of surface-immobilized signaling probes, as shown in FIG. 17. Quantitative analysis of the sensor signal, and therefore accurate determination of target molecule concentration, is based on the fact that the measured faradaic peak height is directly proportional to number of redox moieties immobilized at the electrode, that is, the number of nucleic acid sensor molecules ligated to signaling probes times the multiplicity of redox moieties per signaling probe molecule. Signal generation by the nucleic acid sensor molecules is thus amplified by virtue of multiple redox species per signaling probe. In addition, if an alternating current (AC) bias voltage is applied (superposed) on top of the DC linear voltage ramp applied to the sensor electrodes, i.e., in the case of AC voltammetry, signal amplification would result from the cyclic repetition of the signal-generating redox reaction.

The system described above for the case of a surface-immobilized nucleic acid sensor molecule which ligates a signaling probe containing one or more modified nucleotides conjugated to redox active species suggests a general method and instrumentation for the detection and quantitation of an arbitrary target molecule in solution in real time. Detection of a particular target would require development of a nucleic acid sensor molecule that recognizes the target molecule. Additionally, nucleic acid sensor molecules have been developed which are activated only in the presence of two different target molecules. Such dual-effector sensors could be used to detect the simultaneous presence of two or more targets, or could be used in conjunction with single-target molecule sensors to form biological logic (i.e., AND, OR, etc.) circuits.

Multiplexed detection of multiple target molecules simultaneously in a complex sample solution could be accomplished by immobilizing nucleic acid sensor molecules against the target molecules of interest on separate electrodes within a two-dimensional array of electrodes. A complex sample solution containing multiple target molecules and a common signaling probe could then be introduced to the array. All nucleic acid sensor molecules would be exposed simultaneously to all targets, with the target-activated nucleic acid sensor molecule response(s) being observed and recorded only at the spatial location(s) known to contain a nucleic acid sensor molecule specific for the target molecules present in the (unknown) sample. The utility of such a nucleic acid sensor molecule array would be greatly enhanced by the integration of a microfluidic sample and reagent delivery system. Such an integrated microfluidic system would allow the application of reagents and samples to the sensor array to be automated, and would allow the reduction of sample volume required for analysis to <1 μL.

The sensor array electrodes may be of any configuration, number, and size. In a preferred embodiment, the sensor and reference electrodes would be circular gold pads on the order of 100-500 μM in diameter, separated by a center-to center distance equal to twice their diameter. Each electrode would be addressed by separate electrical interconnects. The application of electrical signals to the sensor electrodes can be accomplished using standard commercially available AC and DC voltage sources. Detection of faradaic electrical signals from the sensor electrodes can be accomplished easily using standard commercially available data acquisition boards mounted within and controlled by a microcomputer. Specifically, the raw sensor current signals would need to be amplified, and then converted to a voltage and analyzed via a high resolution (i.e., 16 bit) analog to digital converter (ADC). It is possible to reduce the signal background and to increase the signal to noise ratio (SNR) by using the common technique of phase-sensitive detection. In this detection method, an alternating current (AC) bias voltage (at a frequency between, for example, 100 to 1000 Hz) is superposed on top of the DC linear voltage ramp applied to the sensor electrodes. The frequency of the applied bias voltage is called the fundamental frequency. It can be shown that the sensor response signal contains multiple frequency components, including the fundamental frequency and its harmonics (integral multiples of the fundamental frequency). It can further be shown that the nth harmonic signal is proportional to the nth derivative of the signal. Detecting these derivative signals (by means of a lock-in amplifier) minimizes the effects of constant or sloping backgrounds, and can enhance sensitivity by increasing the signal to noise ratio and allowing the separation of closely spaced signal peaks. It should be noted that digital, computer-controlled AC and DC voltage sources (i.e., digital to analog converters, DACs), current preamplifiers, analog to digital converters (ADCs), and lock-in amplifiers are all available as integrated signal generation/acquisition boards that can be mounted within and controlled by a single microcomputer.

In a preferred embodiment, an integrated nucleic acid sensor molecule system with electrochemical detection would include the following elements: one, an independently addressable multielement electrode array with immobilized surface layer composed of conductive species separated by insulating species and sensors; two, optical nucleic acid sensor molecules immobilized on the electrode array; three, an oligonucleotide substrate/signaling probe which ligates with the nucleic acid sensor molecule in the presence of the appropriate target; four, an automated or semi-automated microfluidic reagent and sample delivery system; and five, a reader instrument/data acquisition system consisting of a microcomputer controlling the appropriate voltage sources, current and lock-in amplifiers, data acquisition boards, and software interface for instrument control and data collection.

In another embodiment, the change in activity of the nucleic acid sensor molecule can be detected by watching the change in fluorescence of a nucleic acid sensor molecule when it is immobilized on a chip. A ligase can be attached to a chip and its ligase activity monitored. Ligase nucleic acid sensor molecules, labeled with one fluorophore, e.g., Cy3, are attached via an amino modification to an aldehyde chip. The initial Cy3 fluorescence indicates the efficiency of immobilization of the nucleic acid sensor molecules. Next, the chip is exposed to a substrate labeled with a second fluorophore, e.g., CyS, with or without the target. In the presence of target, the nucleic acid sensor molecule ligates the substrate to itself, and becomes Cy5-labeled. Without target, the ligation does not occur.

The use of a labeled effector oligonucleotide does not change the rate of ligation of the nucleic acid sensor molecule whether target is present or not. When using nucleic acid sensor molecules in the context of a chip based system, in one embodiment, an effector oligonucleotide is used to attach the nucleic acid sensor molecule to the chip.

In another embodiment, a hammerhead nucleic acid sensor molecule could be used to measure the concentration of an analyte through the use of fluorescence.

Any optical method known in the art, in addition to those described above can be used in the detection and/or quantification of all targets of interest in all sensor formats, in both biological and nonbiological media.

Any other detection method can also be used in the detection and/or quantification of targets. For example, radioactive labels could be used, including ³²P, ³³P, ¹⁴C, ³H, or ¹²⁵I. Also enzymatic labels can be used including horseradish peroxidase or alkaline phosphatase. The detection method could also involve the use of a capture tag for the bound nucleic acid sensor molecule.

7) Nucleic Acid Sensor Molecules

FIG. 18 illustrates an RNA ribozyme library derived from a hammerhead sequence pool consisting of up to 10¹⁷ variants of randomized sequences appended to the hammerhead ribozyme motif. The starting pool of nucleic acids comprising a target modulation domain (TMD), linker domain (LD) and catalytic domain (CD) (see also FIG. 2) was prepared on a DNA synthesizer. Random nucleotides are incorporated during the synthesis to generate pools of roughly 10¹⁷ molecules. Linker scanning library is designed to identify cis-hammerhead NASMs that are modulated by target. The linker library was generated by appending a target modulation domain to the randomized linker domain to create a library of potential target-modulated cis-hammerhead NASMs. The linker library of target-modulated cis-hammerhead NASMs consists of up to 65,000 variants. Most molecules in the randomized NASM pools are non-functional NASMs. In some libraries, the catalytic site is a known sequence (a ligase site or a hammerhead catalytic core) and is at least a portion of either the 5′ and/or 3′ fixed region (the other portion being supplied by the random sequence), or is a complete catalytic site. In some cases, the catalytic site may be selected along with the target molecule binding activity of oligonucleotides within the oligonucleotide pool.

Sorting among the sensor candidates to find the desired molecules starts from the complex sequence pool, whereby desired target-modulated sensors are isolated through an iterative in vitro selection process: in addition to the target-activated NASMs that one desires, the starting pool is usually dominated by either constitutively active or completely inactive ribozymes. The selection process removes both types of contaminants. In a following amplification stage, thousands of copies of the surviving sequences are generated to enable the next round of selection. During amplification, random mutations can be introduced into the copied molecules—this ‘genetic noise’ allows functional NASMs to continuously evolve and become even better adapted as target-activated enzymes. The entire experiment reduces the pool complexity from 10¹⁷ molecules down to around 100 sensor candidates that require detailed characterization.

The nucleic acid sensor molecules identified through in vitro selection comprise a catalytic domain (i.e., a signal generating moiety), coupled to a target modulation domain, (i.e., a domain which recognizes target and which transduces that molecular recognition event into the generation of a detectable signal). In general, the target modulation domain is defined by the minimum number of nucleotides sufficient to create a three-dimensional structure which recognizes target. In addition, the nucleic acid sensor molecules of the present invention use the energy of molecular recognition to modulate the catalytic or conformational properties of the nucleic acid sensor molecule. The selection process as described in detail in the present invention identifies novel nucleic acid sensor molecules through target modulation of the catalytic core of a ribozyme.

The NASM selection procedures place selective pressure on catalytic effectiveness of potential NASMS by modulating both target concentration and reaction time-dependence. Either parameter, when optimized throughout the selection, can lead to nucleic acid molecular sensor molecules which have custom-designed catalytic properties, e.g., NASMs that have high switch factors, and or NASMs that have high specificity.

Sensor candidates which are derived from in vitro selection are tested as target modulated biosensors. The pool of sensor candidates is cloned into various plasmids transformed into E. coli. Individual sensor encoded DNA clones are isolated, PCR amplified and the sensor candidate is transcribed in vitro to generate sensor RNA. The sensor RNAs are then tested in target modulation assays which determine the rate or extent of ribozyme modulation. For hammerhead sensor RNAs, the extent of target dependent and independent reaction is determined by quantifying the extent of self cleavage of an oligonucleotide substrate in the absence or presence of target. The extent of reaction can be followed by electrophoretic separation of the reaction products on a denaturing PAGE gel, and subsequently analyzed by standard radiometric methods.

Individual sensor clones which display high target dependent switch factor values, or high k_{act} rate values are subsequently chosen for further modification and evaluation. Hammerhead derived NASM clones are then further modified to render them suitable for the optical detection applications that are described in detail below. In brief, these sensors are used as fluorescent biosensors affixed to solid supports, as fluorescent biosensors in homogeneous FRET-based assays.

8) Uses of NASMS

NASMs have been developed for purposes of detection of components in a test mixture (e.g., a sample that contains a plurality of molecules). This includes, but is not limited to, samples from process solutions used in the production of various food stuffs and beverages, bodily fluids, cell cultures, and any sample for environmental and toxicology testing such as contaminated water and industrial effluent. As used herein, “bodily fluid” refers to a mixture of molecules obtained from an organism. This includes, but is not limited to, whole blood, blood plasma, urine, semen, saliva, lymph fluid, meningal fluid, amniotic fluid, glandular fluid, sputum, and cerebrospinal fluid. This also includes experimentally separated fractions of all of the preceding. Bodily fluid also includes solutions or mixtures containing homogenized solid material, such as feces, tissues, and biopsy samples. Examples of beverages include coffee and soft drinks.

NASMs have been described that directly recognize caffeine and aspartame. NASMs, based upon the hammerhead, self-cleaving ribozyme, have been developed which emit a fluorescent signal in the presence of caffeine or aspartame but not structurally related analogs. These sensors can be used to measure the presence of concentration of these targets in platform for high-throughput screening, for example in the format of a biosensor.

Because signal generation in the NASM biosensor is reversible, washing of the biosensor(s) in a suitable buffer will allow the biosensor(s) to be used multiple times, enhancing the reproducibility of the any diagnostic assay since the same reagents can be used repeatedly. Suitable wash buffers include, e.g., binding buffer without target or, for faster washing, a high salt buffer or other denaturing conditions, followed by re-equilibration with binding buffer.

Re-use of the biosensor is enhanced by selecting optimal fluorophores. For example, Alexa Fluor 488, produced by Molecular Probes, has similar optical characteristics compared to fluorescein, but has a much longer lifetime. Another way to re-use biosensors involves engineering a site recognized by a nuclease proximal to the signal generating site, and sequences comprising signaling moieties are removed from the biosensor and replaced by new sequences, as needed.

The invention is further illustrated in the following non-limiting examples.

### EXAMPLES

### Example 1

### Selection Strategies: General

Sensors for both caffeine and aspartame were generated using a panel of in vitro selection strategies. ADP and cGMP selections were also performed as controls.

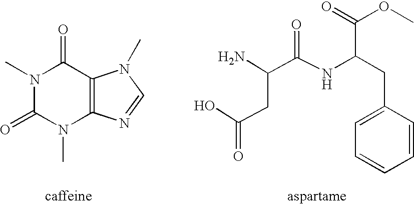

Three separate strategies were employed to generate NASM detection systems for caffeine and aspartame. In brief, strategy one involved first identifying aptamers (based on target affinity) to the target molecules using a standard pool (N₄₀APT), followed by using those aptamer sequences to design NASM molecules. In strategy two, pool molecules comprised of a hammerhead ribozyme core appended with a randomized target binding domain (pool designations HH₃₃WT and HH₃₃AG) were subjected to selection on the basis of target-dependent cleavage activity. In strategy 3, hammerhead based pools were first enriched for binding to caffeine or aspartame, then subjected to selection on the basis of caffeine or aspartame-dependent activity. In addition to de novo selection for caffeine- and aspartame-dependent sensors, known putative caffeine binding sequences were used. Thus, the random regions from several clones isolated from a caffeine binding selection are described in U.S. Pat. No. 5,580,737, which is specifically incorporated herein by reference.

The desired selection schemes required synthesis of three different RNA pools, N₄₀APT, HH₃₃WT, and HH₃₃AG (Eckstein et al., RNA Structure and Function (1998) Cold Spring Harbor Laboratory Press, pg. 341), as described below. The N₄₀APT pool contained sequences with a 5′ oligonucleotide linked to a randomized region of 40 nucleotides which is linked to a 3′ oligonucleotide. The HH₃₃WT, and HH₃₃AG pools contained sequences with a 5′ oligonucleotide linked, to a randomized region of 33 nucleotides which is linked to a 3′ oligonucleotide, and are shown schematically in FIG. 18. Transcription conditions for each pool were optimized and sufficient quantities of RNA to carry all of the selections were prepared. Each selection was initiated with approximately 4×10¹⁵ RNA molecules (6.6 nmoles).

Binding selections are performed by immobilizing the target molecule on an appropriate resin, applying pool RNA to the resin, and collecting only those RNA molecules that are specifically eluted when the resin is washed with buffer containing target free in solution. The RNA pool used for this approach is N₄₀APT, which has been used successfully to identify RNA ligands for small molecules. The aspartame and caffeine resins were prepared as follows:

As shown in Scheme 1, aspartame (Asp-Phe-OMe) was linked via the C-terminal end, reasoning that the methyl ester would not be a key moiety involved in RNA binding interactions. To simplify coupling to solid support, the tripeptide Asp-Phe-Cys (DFC) was used. DFC was efficiently linked to activated thiol Sepharose via a disulfide linkage.

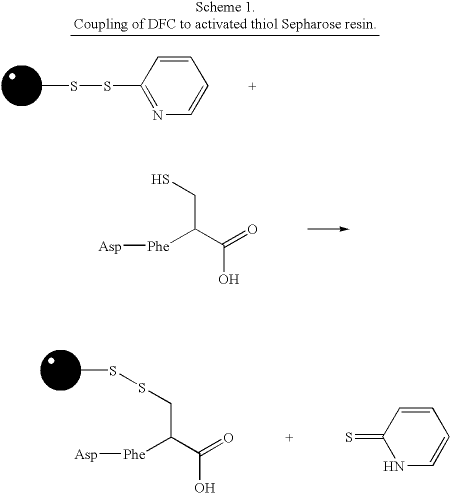

The aspartame (Asp-Phe-OMe) derivative Asp-Phe-Cys was used to facilitate coupling to solid support. The tripeptide was coupled to thiopropyl Sepharose resin (Pharmacia) via a disulfide linkage. 2.6 grams thiopropyl Sepharose powder was suspended in 50 mL water. The resin was washed five times with 40 mL water then three times with 40 mL PBS (pH 7.5) plus 1 mM EDTA. 38 mg Asp-Phe-Cys (99 μmole) was dissolved in 17 mL PBS plus 0.9 mM EDTA, pH 8. The Asp-Phe-Cys solution was added to the resin and the slurry was incubated at room temperature. The progress of the reaction was monitored by removing aliquots and measuring the concentration of the byproduct thiopyridone at 343 nm. After 100 minutes the resin was washed twice with PBS, 1 mM EDTA. The resin was stored at 4° C. as a 60% slurry in PBS with 20% ethanol. The concentration of Asp-Phe-Cys on the resin was 5.5 mM (5.5 μmoles/ₘl resin).

As shown in Scheme 2, caffeine was linked to epoxyaminohexyl (EAH) Sepharose resin using the derivative theophylline-7-acetic acid.

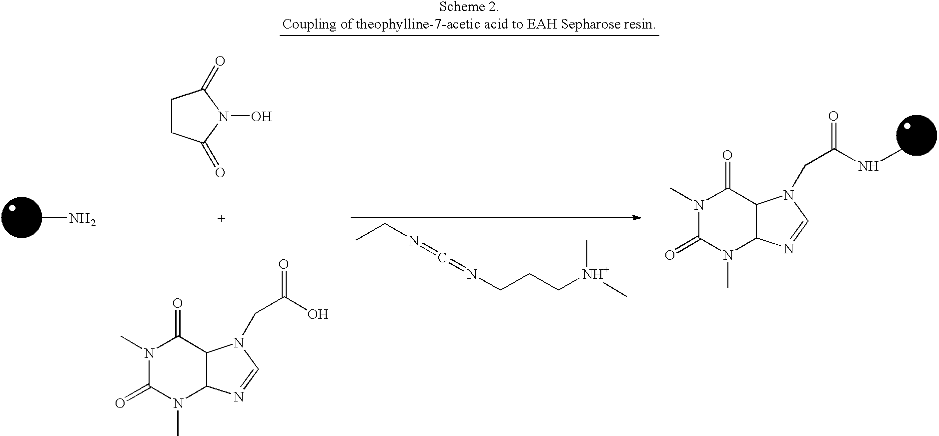

The caffeine derivative theophylline-7-acetic acid was immobilized on epoxyaminohexyl (EAH) Sepharose (Pharmacia). 6.5 mL of EAH sepharose was washed twice with 5 mL PBS pH 7.2. 23 mg (97 nmole) theophylline 7-acetic acid, 66 mg (571 nmole) N-hydroxysuccinimide (NHS), 200 mg 1-ethyl-3-(3-dimethylaminopropyl)carbodiimide hydrochloride (EDC) (104 nmole) were added to 5 mL of a 1:1 dioxane:PBS (pH 7.2) solution. This solution was added to the washed EAH Sepharose resin and the resulting slurry was incubated overnight at room temperature. After removal of the supernatant the resin was washed with 5 mL 1:1 dioxane:PBS (pH 7.2) four times, followed by 5 mL 0.1M sodium acetate pH 5.3:0.1 M sodium chloride and 5 mL 0.1 M sodium hydroxide. The resin was then washed with water until the pH of the supernatant was less than 8. The resin was stored at 4° C. as a 50% slurry in 0.1% SDS. The extent of coupling was determined by treating the derivatized resin with excess 2,4,6-trinitrobenzene sulfonic acid (TNBSA) and measuring the amount of unreacted TNBSA by reaction with glycine and monitoring the reaction product at 335 nm. Antoni et al., Analytical Biochemistry 129:60-63 (1983). The concentration of caffeine on the resin was approximately 8.3 mM (8.3 μmole/mL resin). The reaction scheme for the TNBSA assay used to quantitate loading of theophylline 7-acetic acid on the EAH sepharose resin is shown in Scheme 3.

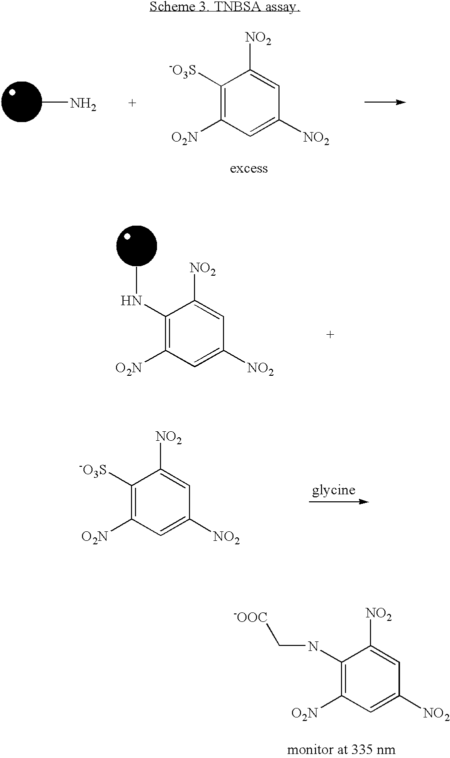

### Example 2

### Selection Strategies: Strategy 2

The second strategy utilized selection on the basis of activity. A pool of molecules which included a hammerhead ribozyme core appended to a randomized target modulation domain was first incubated in reaction buffer in the absence of the desired target. Ribozymes that cleave under these conditions are isolated and discarded. Ribozymes that do not cleave are isolated and then incubated in the presence of the desired target. Those molecules that cleave in the presence of the target are isolated and amplified for use in the next round of selection. The pool used for this selection strategy was HH₃₃WT. During selection, cleaved and uncleaved ribozyme were separated using denaturing gel electrophoresis and the percent cleavage was quantified. When an activity-based selection is nearing completion, substantially more cleavage is observed in the presence of target than in the absence of target.

The DNA templates JD.05.100.A: 5′-AAAGGGCAACCTACGGCTTTCACCGTTTCG-3′ (SEQ ID NO: 16)—N₃₃-5′-CTCATCAGGGTCGCCCTATAGTGAGTCGTATTA-3′ (SEQ ID NO:17), encoding the HH₃₃WT pool, and STC.12.142.A: 5′-AAAGGGCAACCCACGGCTTTCACCGTTTCG-3′ (SEQ ID NO: 18)—N₃₃-5′-CTCATCAGGGTCGCCCTATAGTGAGTCGTATTA-3′ (SEQ ID NO:17) encoding the HH₃₃AG pool, were synthesized on an Applied Biosystems Expedite on a 1 μmole scale using standard phosphoramidite chemistry. The DNA oligonucleotides were purified on Poly-Pak II cartridges (Glen Research) using the protocol recommended by the supplier.

RNA pool molecules for the first round of selection were prepared via run off transcription using T7 RNA polymerase. 1 mL of a mixture containing 10 nmole of STC.12.142A or JD.05.100.A, 15 nmole STC.12.143.A 5′-TAATACGACTCACTATAGGGCGACCCTGATGAG-3′. (SEQ ID NO: 19), 10 mM Tris pH 8, 1 mM EDTA and 100 mM NaCl was heated to 95° C. for 3 minutes followed by rapid cooling on ice. The hybridized templates were then added to transcription mixtures containing 40 mM Tris pH 7.8, 25 mM MgCl₂, 1 mM spermidine, 0.1% triton X-100, 5 mM each NTP, 40 mM DTT, and 50,000 units T7 RNA polymerase. The reactions were incubated overnight at 37° C., quenched with 50 mM EDTA, ethanol precipitated then purified on 3 mm denaturing polyacrylamide gels (8 M urea, 10% acrylamide; 19:1 acrylamide:bisacrylamide). Each 20 mL transcription yielded approximately 150 nmoles of pool RNA. Prior to use, the pool RNA was treated with RQ 1 DNA polymerase (Promega) under the conditions prescribed by the manufacturer to remove template DNA.

Molecules were selected on the basis of their ability to undergo self-cleavage in the presence of caffeine or aspartame. Each round of the selection procedure involved two steps: negative selection in the absence of target and positive selection in the presence of target. A negative selection step was not included in the first round of selection.

A. Caffeine Selections:

The selection was initiated by incubation of 4×10¹⁵ molecules of pool RNA (approximately 3 μM) in selection buffer (150 mM NaCl, 10 mM MgCl₂, 1 mM EDTA, 10 mM sodium phosphate, 90 mM Hepes, pH 7.1) in the presence of 5 mM caffeine at room temperature for 135 minutes. The reaction was quenched by the addition of 25 mM EDTA and 140 mM NaCl, followed by addition of 1 volume isopropanol. After centrifugation, the RNA pellet was resuspended in 40% formamide, 2.5 mM EDTA, heated at 90° C. for 3 minutes then loaded onto a 10% denaturing acrylamide gel (1.5 mm). The band representing cleavage product was removed from the gel by electroelution in an Elutrapg apparatus (Schleicher and Schuell) at 225V for 1 hour in 1×TBE (90 mM Tris, 90 mM boric acid, 0.2 mM EDTA). The eluted material was precipitated by the addition of 300 mM sodium acetate and 1 volume isopropanol.

Reverse transcription was carried out essentially as prescribed by the Invitrogen ThermoScript RT-PCR™ system instructions. The RNA pellet was resuspended in 45 μL water and combined with 1.2 nmole primer STC.12.143.B: 5′-AAAGGGCAACCTACGGCTTTCACCGTTTC-3′ (SEQ ID NO:20), 1.6 mM each dNTP in a total volume of 150 μL aliquoted into 5 separate 0.2 mL PCR tubes. This mixture was heated at 65° C. for 5 minutes followed by rapid cooling to 4° C. Reverse transcriptase and buffer components were added and the mixture was incubated for 30 minutes at 50° C. (50 mM Tris acetate pH 8.4, 75 mM KOAc, 8 mM Mg(OAc)₂, ˜1 mM dNTP, 5 mM DTT, 2 units/μL RNaseOUT™, 0.75 units/μL ThermoScript RT, unidentified stabilizer). The RT mixture was added directly to the PCR reaction mix (20 mM Tris pH 8.4, 50 mM KCl, 2 mM MgCl₂, 0.5 μM STC.12.143.A, 0.5 μM STC.12.143.B, 0.5 mM each dNTP, 0.05 units/μL taq polymerase, 2 mL final volume) and amplified by 8 cycles of PCR (94° C. 1 min, 55° C. 1 min, 72° C. 1 min). The mixture was then phenol:chloroform extracted and ethanol precipitated. Half of the product DNA (1.5 nmoles) was used as a template for the production of round 2 RNA.

Transcription of round 2 pool RNA was carried out overnight at 37° C. (0.75 μM template, 40 mM Tris pH 7.8, 25 mM MgCl₂, 1 mM spermidine, 0.1% triton X-100, 5 mM each NTP, 40 mM DTT, 2,000 units T7 RNA polymerase). The reaction was quenched with 50 mM EDTA, ethanol precipitated then purified on a 1.5 mm denaturing polyacrylamide gels (8M urea, 10% acrylamide; 19:1 acrylamide:bisacrylamide).

Subsequent rounds were carried using a similar procedure, but including a negative selection step. Pool RNA (˜3 μM) was incubated in selection buffer at room temperature for a fixed period. The reaction was quenched by the addition of 25 mM EDTA, 150 mM NaCl, and 1 volume isopropanol. After precipitation, the RNA pellet was resuspended in 40% formamide, 2.5 mM EDTA, heated at 90° C. for 3 minutes then loaded onto a 10% denaturing acrylamide gel (1.5 mm). The band representing full length pool RNA was removed from the gel by electroelution in an Elutrap® apparatus at 225V for 1 hour in 1×TBE. The eluted material was precipitated by the addition of 300 mM sodium acetate and 1 volume isopropanol. For the positive selection step, the pool RNA carried from the negative step was incubated in selection buffer plus 5 mM caffeine for a fixed period. The reaction was quenched by the addition of 25 mM EDTA, 150 mM NaCl, and 1 volume isopropanol. After precipitation, the RNA pellet was resuspended in 40% formamide, 2.5 mM EDTA, heated at 90° C. for 3 minutes then loaded onto a 10% denaturing acrylamide gel (1.5 mm). The band representing cleaved pool RNA was removed from the gel by electroelution in an Elutrap® apparatus at 225V for 1 hour in 1×TBE. The eluted material was precipitated by the addition of 300 mM sodium acetate and 1 volume isopropanol.

Reverse transcription was carried out essentially as prescribed by the Invitrogen ThermoScript RT-PCR™ system instructions. The RNA incubated with an excess of primer STC.12.143.B (SEQ ID NO:20) and 1.6 mM each dNTP at 65° C. for 5 minutes followed by rapid cooling to 4° C. Reverse transcriptase and buffer components were added and the mixture was incubated for 30 minutes at 50° C. (50 mM Tris acetate pH 8.4, 75 mM KOAc, 8 mM Mg(OAc)₂, ˜1 mM dNTP, 5 mM DTT, 2 units/μL RNaseOUT™, 0.75 units/μL ThermoScript RT, unidentified stabilizer). The RT mixture was added directly to a PCR reaction mix (20 mM Tris pH 8.4, 50 mM KCl, 2 mM MgCl₂, 0.5 μM STC.12.143.A, 0.5 μM STC.12.143.B, 0.5 mM each dNTP, 0.05 units/μL taq polymerase) and amplified by PCR (94° C. 1 min, 55° C. 1 min, 72° C. 1 min; in later rounds, the incubation times were reduced to 30 seconds). The PCR product was purified using a QIAquick PCR purification kit (Qiagen), following the manufacturer's instructions. Approximately half of the DNA template was used for transcription of pool RNA for the subsequent round. Transcription was carried out overnight at 37° C. (0.75 μM template, 40 mM Tris pH 7.8, 25 mM MgCl₂, 1 mM spermidine, 0.1% triton X-100, 5 mM each NTP, 40 mM DTT, ˜1 units/μL T7 RNA polymerase, 15 μCi α-³²P UTP). The transcriptions were quenched with 50 mM EDTA, ethanol precipitated then purified on 1.5 mm denaturing polyacrylamide gels (8M urea, 10% acrylamide; 19:1 acrylamide:bisacrylamide). Full length product RNA was removed from the gel by electroelution in an Elutrap® apparatus at 225V for 1 hour in 1×TBE. The eluted material was precipitated by the addition of 300 mM sodium acetate and 1 volume isopropanol.

Beginning in round four, the protocol was modified such that the pool RNA would undergo three cycles of denaturation and refolding during each negative selection step. Briefly, pool RNA (˜3 μM) was incubated in selection buffer at room temperature for a fixed period. The reaction was quenched by the addition of 25 mM EDTA, 300 mM NaOAc, and 1 volume isopropanol. After precipitation, the pellet was resuspended either in 90 μL H₂O followed by the addition of 10 μL 100 mM NaOH or directly in 10 PL 100 mM NaOH to denature the RNA. The reaction was neutralized by the addition of 12 μl 3M NaOAc, and the RNA was precipitated by the addition of one volume isopropanol. The pellet was resuspended in selection buffer and the above protocol was repeated such that in total the pool RNA was subjected to three incubations in selection buffer and two denaturation steps during each negative selection step. The positive selection steps were conducted as described above with one modification; the positive selection reactions were quenched with 25 mM EDTA and 300 mM NaOAc.

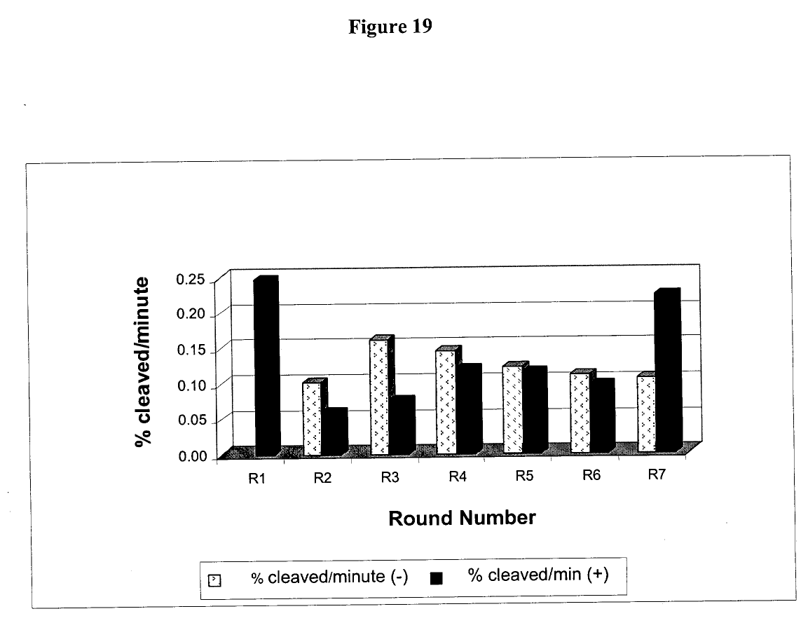

The % cleavage in both the negative (− target) and positive (+ target) steps of each early round of selection for caffeine are plotted in FIG. 19.

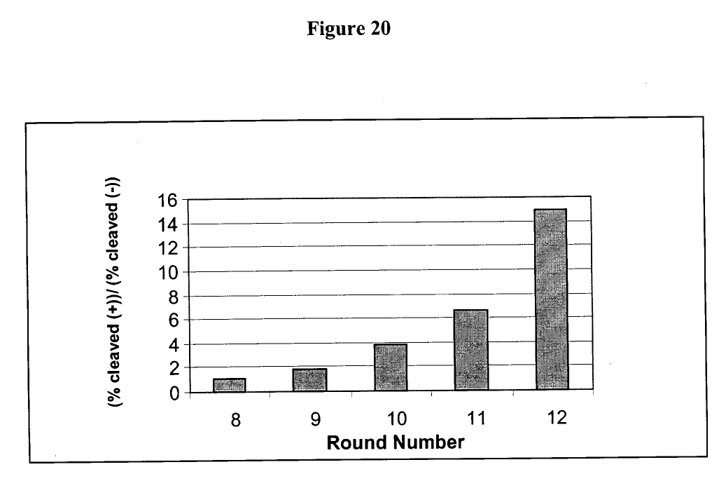

Beginning in round 8, the activity of the post-negative step pool RNA in the presence and absence of 5 mM caffeine was measured during each round. The RNA pellet resulting from the negative selection step was resuspended in 145 μL selection buffer. 125 μL of the solution was carried forward into the positive selection. The remaining 20 μL was divided quickly into two 10 μL aliquots. To one aliquot, 10 μL selection buffer was added and to the other was added 10 μL selection buffer plus 5 mM caffeine. These reaction mixtures were incubated for a fixed period, then quenched with 25 mM EDTA and 300 mM NaOAc and precipitated with one volume of isopropanol. After precipitation, the RNA pellet was resuspended in 40% formamide, 2.5 mM EDTA, heated at 90° C. for 3 minutes then loaded onto a 10% denaturing acrylamide gel (1.5 mm). The extent of cleavage in the two reactions was quantitated using a Storm Phosphoimager (Molecular Dynamics). The progress of the selection was monitored by calculating the ratio of the extent of cleavage in the presence of 5 mM caffeine vs. the extent of cleavage in the absence of caffeine. When the pool exhibits caffeine-dependent activity, this ratio exceeds one. The plot in FIG. 20 shows that between selection rounds 8 and 12 significant caffeine-dependent activity emerged. The percent cleaved per minute is a ratio of the negative (− target) and positive (+ target) cleavage reactions. A ratio greater than 1 indicates increased activity in the presence of target.

Following round 12, the pool template was cloned using the TOPO TA cloning kit (Invitrogen) following the manufacturer's instructions. 18 colonies were isolated and the inserts were amplified by PCR (20 mM Tris pH 8.4, 50 mM KCl, 2 mM MgCl₂, 0.5 μM STC.12.143.A, 0.5 μM STC.12.143.B, 0.5 mM each dNTP, 0.05 units/μL taq polymerase). The resulting template DNAs were purified using a QIAquick PCR purification kit. Template DNA was used to program run-off transcription (˜0.75 μM template, 40 mM Tris pH 7.8, 25 mM MgCl₂, 1 mM spermidine, 0.1% triton x-100, 5 mM each NTP, 40 mM DTT, ˜1 units/μL T7 RNA polymerase, 5 μCi α-³²P UTP) mixtures, incubated overnight at 37° C. The transcriptions were quenched with 50 mM EDTA, ethanol precipitated then purified on 1.5 mm denaturing polyacrylamide gels (8M urea, 10% acrylamide; 19:1 acrylamide:bisacrylamide). Full length product RNA was removed from the gel by electroelution in an Elutrap® apparatus at 225V for 1 hour in 1×TBE. The eluted material was precipitated by the addition of 300 mM sodium acetate and 1 volume isopropanol.

After pool templates produced from rounds 11 and 12 of selection were cloned, individual clones were sequenced at LARK Technologies, Inc. The sequences obtained are listed in Table 1.

TABLE 1

Caffeine NASMS

| | | |
|---|---|---|
| | | SEQ |
| | | ID |
| Identifier | Sequence | NO |

| | | |
|---|---|---|
| Round 11 | | |
| ARX9P1.A05 | GGGCGACCCTGATGAGCATCCCAGCCTCTGTCGCTGAGGCCGTGAAGATCGAAACGGTGA | 21 |
| | AAGCCGTAGGTTGCCCA | |
| ARX9P1.A06 | GGGCGACCCTGATGAGGACGAACGCCAATGGCCAGTTCGAGTAAGACCGCGAAACGGTGA | 22 |
| | AAGCCGTAGGTTGCCCA | |
| ARX9P1.B02 | GGGCGACCCTGATGAGCAGCCGGATCTGATCTACTCTAGACTCTGAGCTCGAAACGGTGA | 23 |
| | AAGCCGTAGGTTGCCCTTT | |
| ARX9P1.B04 | GGGCGACCCTGATGAGCGGGCATGTGGTAAGAGCGTTCCTGCATGACCCCGAAACGGTGA | 24 |
| | AAGCCGTAGTTGCCC | |
| ARX9P1.B06 | GGGCGACCCTGATGAGGTATGCGTGCTTGCATGATTTCGGGCACCTTCGCGAAACGGTGA | 25 |
| | AAGCCGTAGGTTGCCCTTT | |
| ARX9P1.C01 | GGGCGACCCTGATGAGCAGCCGAGATCTGACGAACACGAGCTCAGGTTCGAAACGGTGAA | 26 |
| | AGCCGTAGGTTGCCCTTT | |
| ARX9P1.C04 | GGGCGACCCTGATGAGGATGCGACCACTCTGCGGTCGTGAGTAAGCACGCGAAACGGTGA | 27 |
| | AAGCCGTAGGTTGCCCTTT | |
| ARX9P1.C05 | GGGCGACCCTGATGAGGGTACCACGACCATCCTCTGCGTGGTGCCGTGGCGAAACGGTGA | 28 |
| | AAGCCGTAGGTTGCCCTTT | |
| ARX9P1.D02 | GGGCGACCCTGATGAGTCGGATTACTGTGCCTCCCCCGATACCCTGCGCCGAAACGGTGA | 29 |
| | AAGCCGTAGGTTGCCCTTT | |
| ARX9P1.E01 | GGGCGGACCCTGATGAGCAGCCTCGACCCGACCGCGATGGGTAGTGAGTTCGAAACGGTG | 30 |
| | AAAGCCGTAGGTTGCCCTTT | |
| ARX9P1.E03 | GGGCGACCCTGATGAGGTGTTTGTGAAGCCGTGAGCTCCTACCGTGTTCCGAAACGGTGA | 31 |
| | AAGCCGTAGGTTGCCCTTT | |
| ARX9P1.E04 | GGGCGACCCTGATGAGAGACCCAGTATGGGATGAGCCAACTCTGTTGGTCGAAACGGTGA | 32 |
| | AAGCCGTAGGTTGCCCTTT | |
| ARX9P1.E05 | GGGCGACCCTGATGAGTCCGCCAGAGGTGCTCGTCCCCTTCGGCAAGGTCGAAACGGTGA | 33 |
| | AAGCCGTAGGTTGCCCTTT | |
| ARX9P1.F04 | GGGCGACCCTGATGAGCACGCACGCCGGATTCACTCTCCGACATGACGTCGAAACGGTGA | 34 |
| | AAGCCGTAGGTTGCCCT | |
| ARX9P1.G01 | GGGCAGACCCTGATGAGGACCACGCCTTGACGTGCGATGGAGTAAGTACGCGAAACGGTG | 35 |
| | AAAGCCGTAGGTTGCCCTTT | |
| ARX9P1.G02 | GGGCGACCCTGATGAGCACCTCCAGAATCGAACAGCTCGGCTAAAGGTCGAAACGGTGAA | 36 |
| | AGCCGTAGGTTGCCCTTT | |
| ARX9P1.G03 | GGGCGACCCTGATGAGCACCGCGGCTAGGCCTACGTCCCTCCCGCAGGTCGAAACGGTGA | 37 |
| | AAGCCGTAGGTTGCCCTTT | |
| ARX9P1.G04 | GGGCGACCCTGATGAGGACCAAGCCCCGGTGAGCTTTGGAGTAAGATCGCGAAACGGTGA | 38 |
| | AAGCCGTAGGTTGCCAAGGGT | |
| ARX9P1.G05 | GGGCGACCCTGATGAGCACCCGGACCCCAACCGGTGGTCTAGCTTAGGTCGAAACGGTGA | 39 |
| | AAGCCGTAGGTTGCCCTTT | |
| ARX9P1.G06 | GGGCGACCCTGATGAGGACAGGGAGTGTGATGTCCCTGAGTAAGACCGCGAAACGGTGAA | 40 |
| | AGCCGTAGGTTGCCCTTT | |
| ARX9P1.H01 | GGGCGACCCTGATGAGATGGACCGACGAGCAATGTCGCGCACCGAGTCCCGAAACGGTGA | 41 |
| | AAGCCGTAGGTTGCCCTTT | |
| ARX9P1.H02 | GGGCGACCCTGATGAGCGGGACTGGCCGAGCATCTCTGCCTACGCGACCCGAAACGGTGA | 42 |
| | AAGCCGTAGGTTGCCCTTT | |
| ARX9P1.H03 | GGGCGACCCTGATGAGAACGCCTCCATGCCGCTCGGAGGAGGAGTGACGAAACGGTGAAA | 43 |
| | GCCGTAGGTTGCCCTTT | |
| ARX9P1.H04 | GGGCGACCCTGATGAGGACGAGCTAGTCCATCCCAAACGGTGAATGCGACGAAACGGTGA | 44 |
| | AAGCCGTAGGTTGCCCTTT | |
| ARX9P1.H05 | GGGCGACCCTGATGAGGATCCCACAACGCGTGAGTGGGAAGTAAGACCGCGAAACGGTGA | 45 |
| | AAGCCGTAGGTTGCCCTTT | |
| ARX9P1.A01 | GGGCGACCCTGATGAGATAGATCCGTTTACCTACCGATGGTCGAGGTCTCGAAACGGTGA | 46 |
| | AGCCGTAGGTTGCCCTTT | |
| ARX9P1.F06 | GGGCGACCCTGATGAGGCGTGACCGATAACATCTCCGTCACAAGAAGCGCGAAACGGTGA | 47 |
| | AAGCCGTAGGTTGCCCTTT | |
| ARX9P1.D04 | GGGCGACCCTGATGAGGCTCTAGGCTCATGGCCAGTAGGTAAGAACGCGAAACGGTGAAA | 48 |
| | GCCTAGGTTGCCCAAGGGCT | |
| Round 12 | | |
| ARX9P1.A08 | GGGCGACCCTGATGAGCAGAACGTGCGACATGGAATGGCATATGAATCTCGAAACGGTGA | 49 |
| | AAGCCGTAGGTTGCCCTTT | |
| ARX9P1.A09 | GGGCGACCCTGATGAGCGGAGCAATGCCATGATGTGCTGCGGCAACTCCCGAAACGGTGA | 50 |
| | AAGCCGTAGGTTGCCCTTT | |
| ARX9P1.A11 | GGGCGACCCTGATGAGCACCCCGGTCAGCGAGCCTAGATCGAGTTTGGTCGAAACGGTGA | 51 |
| | AAGCCGTAGGTTGCCCTTT | |
| ARX9P1.B10 | GGGCGACCCTGATGAGGACCCACACCCAACAGGAGTGGGAGTAAGACCGCGAAACGGTGA | 52 |
| | AAGCCGTAGGTTGCCCTTT | |
| ARX9P1.B11 | GGGCGACCCTGATGAGCAACCGACAGCGGCGTTACCCGATGTCCCGTGTCGAAACGGTGA | 53 |
| | AAGCCGTAGGTTGCCCTTT | |
| ARX9P1.C07 | GGGCGACCCTGATGAGCAGCCCACGCAGGTCAGCCAACCGAGTGGAGTTCGAAACGGTGA | 54 |
| | AAGCCGTAGGTTGCCCTTT | |
| ARX9P1.C10 | GGGCGACCCTGATGAGCGTGAAGCAGAGCCCTCCCGTCTCTGTGGACGTCGAAACGGTGA | 55 |
| | AAGCCGTAGGTTGCCCTT | |
| ARX9P1.C11 | GGGCGACCCTGATGAGATGCAAGCTAAGCACCGTACCACATAGCATTGCCGAAACGGTGA | 56 |
| | AAGCCGTAGGTTGCCC | |
| ARX9P1.C12 | GGGCGACCCTGATGAGCAGACACGCACTCACTACTCTCTGCGGAGATCTCGAAACGGTGA | 57 |
| | AAGCCGTAGGTTGCCC | |
| ARX9P1.D07 | GGGCGACCCTGATGAGGGTTGCGGGTAGCCGCTATTACCGCGACCGTGGCGAAACGGTGA | 58 |
| | AAGCCGTAGGTTGCCCTTT | |
| ARX9P1.D11 | GGGCGACCCTGATGAGGACATCGCCGTTGCTGCTTTGCGACGGGATTCGCGAAACGGTGA | 59 |
| | AAGCCGTAGGTTGCCC | |
| ARX9P1.D12 | GGGCGACCCTGATGAGCAGACCACGGGTCCTTTGTCTCGGAGACAGTCTCGAAACGGTGA | 60 |
| | AAGCCGTAGGTTGCCC | |
| ARX9P1.E08 | GGGCGACCCTGATGAGGAGCAGCTGTCTACTTGTGGGACGAAGATAAGCTCGAAACGGTG | 61 |
| | AAAGCCGTAGGTTGCCCTTT | |
| ARX9P1.E10 | GGGCGACCCTGATGAGCACGCGCATGCTGCATGGACAAGCCATCGACGTCGAAACGGTGA | 62 |
| | AAGCCGTAGGTTGCCCTT | |
| ARX9P1.E11 | GGGCGACCCTGATGAGCACCCACCGCCCACCTCAAGGCGACAGATTGGTCGAAACGGTGA | 63 |
| | AAGCCGTAGGTTGCCCTTT | |
| ARX9P1.E12 | GGGCGACCCTGATGAGTCCCAGCCNNTCGCGCAAGGNATGAGCCACGGTCGAAACGGTGA | 64 |
| | AACCGTANGTTGCCCATTT | |
| ARX9P1.F07 | GGGCGACCCTGATGAGGACCCTGCCCGCGATGCGGAGGGAGTAAGATCGCGAAACGGTGA | 65 |
| | AAGCCGTAGGTTGCCCTTT | |
| ARX9P1.F10 | GGGCGACCCTGATGAGTGCCCCCGCACATCATAGCGTGGAATAGGCTACGAAACGGTGAA | 66 |
| | AGCCGTAGGTTGCCCTTT | |
| ARX9P1.F12 | GGGCGACCCTGATGAGGACTTCCCCCTACGCTTGGAAGAGTAAGATCGCGAAACGGTGAA | 67 |
| | AGCCGTAGGTTGCCCTTT | |
| ARX9P1.G07 | GGGCGACCCTGATGAGCCTCCATCCGAGGGGGATGTCCCACGATAGGACCGAAACGGTGA | 68 |
| | AAGCCGTAGGTTGCCCTTT | |
| ARX9P1.G12 | GGGCGACCCTGATGAGCGGGCGGGATACGTGTGTTCTATCCATGAACCCCGAAACGGTGA | 69 |
| | AAGCCGTAGGTTGCCCTTT | |
| ARX9P1.H11 | GGGCGACCCTGATGAGGACTCAAGCCGCAGTGCCTTGAGAGTAAGACCGCGAAACGGTGA | 70 |
| | AAGCCGTAGGTTGCCCTTT | |
| ARX9P1.A07 | GGGCGACCCTGATGAGTAGCGCCATTGCCCTACGTCGCTGTAGACTGGACGAAACGGTGA | 71 |
| | AAGCCGTAGGTTGCCCTTT | |

The activity of the individual clones was measured by incubating the RNA (˜1 μM) in selection buffer in the presence or absence of 5 mM caffeine at room temperature for 20 minutes. The reactions were quenched by the addition of 25 mM EDTA, 300 mM NaOAc, and 1 volume isopropanol. After precipitation, the RNA pellet was resuspended in 40% formamide, 2.5 mM EDTA, heated at 90° C. for 3. minutes then loaded onto a 10% denaturing acrylamide gel (1.5 mm). The extent of cleavage in the two reactions was quantitated using a Storm Phosphoimager (Molecular Dynamics). Twelve additional clones that emerged from Round 12 of allosteric selection were tested for caffeine-dependent activity. Clones highly activated by caffeine-dependent were sequenced, as shown in Table 2.

TABLE 2

Additional Caffeine clones.

| | | |
|---|---|---|
| | | SEQ |
| | | ID |
| Identifier | Sequence | NO |

| | | |
|---|---|---|
| ARX12P2.D1 | GGGCGACCCTGATGAGGATCATCGGACTTTGTCCTGTGGAGTAAGATCGCGAAACGGTGAAA | 72 |
| 1 | GCCGTAGGTTGCCCTTT | |
| ARX12P2.B1 | GGGCGACCCTGATGAGGACGTCATGGAGTCGACATGACGAGTAAGACCGCGAAACGGTGAAA | 73 |
| 2 | GCCGTAGGTTGCCCTTT | |
| ARX12P2.A1 | GGGCGACCCTGATGAGGACCGGATGGCTGGCCCATTCGGCGTAAGACCGCGAAACGGTGAAA | 74 |
| 1 | GCCGTAGGTTGCCCTTT | |
| ARX12P2.F1 | GGGCGACCCTGATGAGGACACTAGGTCACTTCCCTAGTGAGTAAGACCGCGAAACGGTGAAA | 75 |
| 2 | GCCGTAGGTTGCCCTTT | |

The results of the activity of various clones are summarized in Table 3. The switch factor is defined as the percent cleavage observed in the presence of 5 mM caffeine divided by the percent cleavage observed in the absence of caffeine. The caffeine pool was cloned, and individual sequences were tested for caffeine-dependent activity. In a 20 minute assay, 11 out of 18 clones were activated more than 10-fold by 5 mM caffeine.

TABLE 3
Switch factors for selected caffeine sensors.

| | Clone identifier | |
|---|---|---|
| Clone identifier (LARK) | (internal) | Switch factor^{a} |
| ARX12P2.A11 | STC.43.29.D7 | 149.8 |
| | STC.43.29.D8 | n.d. |
| | STC.43.29.D9 | 11.3 |
| | STC.43.29.D10 | 13.3 |
| ARX12P2.D11 | STC.43.29.D11 | 40.1 |
| | STC.43.29.D12 | 5.7 |
| ARX12P2.B12 | STC.43.29.E7 | 81.2 |
| ARX12P2.F12 | STC.43.29.E8 | 33.7 |
| | STC.43.29.E9 | 26.3 |
| | STC.43.29.E10.E11 | 2.0 |
| | STC.43.29.E12 | 10.5 |
| ARX9P1.B04 | STC.43.46.C1 | 3.0 |
| ARX9P1.B10 | STC.43.46.C2 | 54.3 |
| ARX9P1.H11 | STC.43.46.C3 | 35.5 |
| ARX9P1.C4 | STC.43.46.C4 | 3.2 |
| ARX9P1.E1 | STC.43.46.C5 | 8.7 |
| ARX9P1.F10 | STC.43.46.C9 | 1.5 |
| ARX9P1.A11 | STC.43.46.C11 | 10.7 |

^{a}Measured in a single time point 20 minute assay using a gel-based readout; 1 × selection buffer, 5 mM caffeine. n.d. = the background was to low to permit accurate calculation of a switch factor.

The sequences for the caffeine sensors displaying switch factors greater than 10 are listed in Table 4. Each of these sequences contains a conserved motif which is highlighted.

[image]

The most active clones were then evaluated in the sequence context required for the solution-based FRET assay (see below).

B. Aspartame Selections:

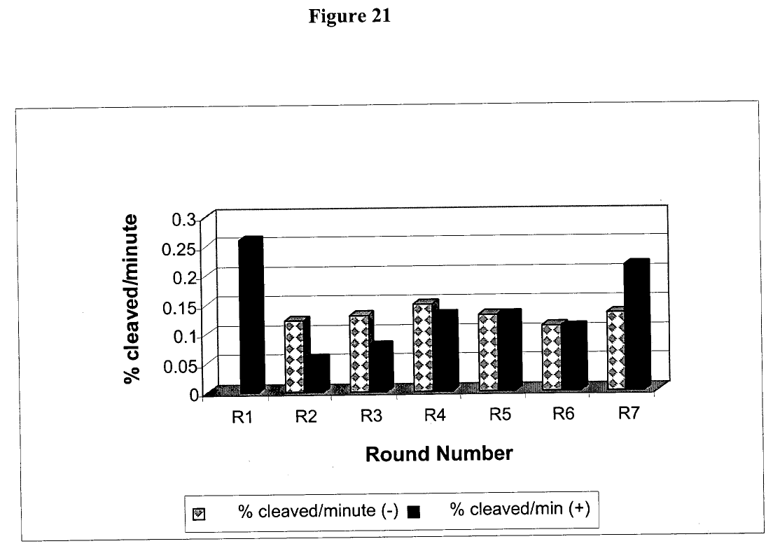

The selections for nucleic acid sensor molecules which were selective for aspartame were performed as described under section A for caffeine NASMs. The % cleavage in both the negative (− target) and positive (+ target) steps of each early round of selection are plotted in FIG. 21.

### Example 3

### Selection Strategies: Strategy 3

The third strategy combined both selection for target binding and selection for target-dependent activity. This strategy involves a two phase selection: a first selection for RNA molecules with the hammerhead motif that bind to caffeine or aspartame, followed by selection for activation of cleavage activity by those molecules. In this approach, pools that contain random sequences in a hammerhead context, HH₃₃WT and HH₃₃AG, were first subjected to several rounds of binding-based selection using the protocol described for strategy one. The pools that emerge were predicted to contain RNA molecules that bind the target molecule and possess hammerhead ribozyme activity.

I. Binding Selections

A. Caffeine Binding Selections

This selection involved enrichment of the pool for molecules with the hammerhead motif that bind to caffeine, followed by selection for activation of cleavage activity by caffeine. Two pools, HH₃₃WT and HH₃₃AG, were used.

The binding-based portion of the selection carried out using the following procedure. Caffeine resin slurry was added to a disposable 1 cm column and allowed to settle to a bed volume of approximately 300 μL. The resin was washed 3 times with 500 μl of selection buffer (500 mM NaCl, 10 mM MgCl₂, 1 mM EDTA, 10 mM sodium phosphate, 90 mM Hepes, pH 7.5) then washed 3 times with 5 ml of selection buffer plus 10 μg/ml tRNA. Pool RNA (20 μM in round 1, ˜2 μM in subsequent rounds) in 300 μL selection buffer was added to the resin and incubated for 5 minutes. The resin was then washed approximately 12 times with 300 μL of selection buffer. Each wash fraction was incubated on the resin for 3 minutes. The resin was next treated with 6-7300 μL washes with selection buffer containing 5 mM caffeine to elute any RNA molecules bound to the immobilized caffeine. All fractions were quantitated using the Bioscan QC 2000 counter (BioScan, Inc., Washington, D.C.). In rounds subsequent to round 1, the resin and sample volumes were reduced to 200 μL and the pool RNA was first passed through a pre-column composed of acetylated EAH resin to remove matrix binding RNAs. The elution fractions were combined and 25 mM EDTA, 40 μg glycogen and 1 volume of isopropanol were added.

Reverse transcription was carried out essentially as prescribed by the Invitrogen ThermoScript RT-PCR™ system instructions. The RNA incubated with an excess of primer STC.12.143.B 5′-AAAGGGCAACCTACGGCTTTCACCGTTTC-3′ (SEQ ID NO:20) for the HH₃₃WT pool or STC.12.143.C₅′-AAAGGGCAACCCACGGCTTTCACCGTTTC-3′ (SEQ ID NO:79) for the HH₃₃AG pool and 1.6 mM each dNTP at 65° C. for 5 minutes followed by rapid cooling to 4° C. Reverse transcriptase and buffer components were added and the mixture was incubated for 30 minutes at 50° C. (50 mM Tris acetate pH 8.4, 75 mM KOAc, 8 mM Mg(OAc)₂, ˜1 mM dNTP, 5 mM DTT, 2 units/μL RNaseOUT™, 0.75 units/μL ThermoScript RT, unidentified stabilizer). The RT mixture was added directly to a PCR reaction mix (20 mM Tris pH 8.4, 50 mM KCl, 2 mM MgCl₂, 0.5 mM STC.12.143.A, 0.5 μM STC.12.143.B (or STC.12.143.C), 0.5 mM each dNTP, 0.05 units/IL taq polymerase) and amplified by PCR (95° C. 1 min, 55° C. 1 min, 72° C. 1 min; in later rounds, the incubation times were reduced to 30 seconds).

The PCR product was purified using a QIAquick PCR purification kit (Qiagen), following the manufacturer's instructions. Approximately one quarter of the DNA template was used for transcription of pool RNA for the subsequent round. Transcription pool RNA was carried out overnight at 37° C. (0.75 μM template, 40 mM Tris pH 7.8, 25 mM MgCl₂, 1 mM spermidine, 0.1% triton x-100, 5 mM each NTP, 40 mM DTT, ˜1 units/μL T7 RNA polymerase, 10 μCi α-³²P UTP). The transcriptions were quenched with 50 mM EDTA, ethanol precipitated then purified on 1.5 mm denaturing polyacrylamide gels (8M urea, 10% acrylamide; 19:1 acrylamide:bisacrylamide). Full length product RNA was removed from the gel by electroelution in an Elutrap® apparatus at 225V for 1 hour in 1×TBE. The eluted material was precipitated by the addition of 300 mM sodium acetate and 1 volume isopropanol.

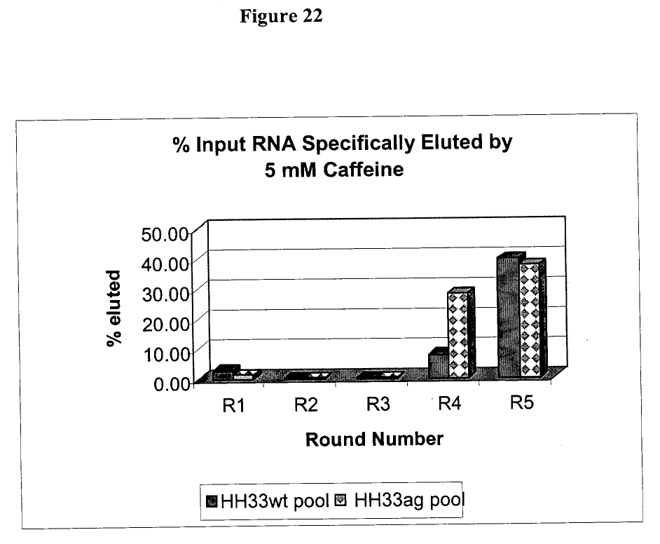

As shown in FIG. 22, both pools were enriched for binding to caffeine after several rounds of selection. The percent of pool RNA specifically eluted from caffeine-derivatized resin is plotted vs. round of selection. For each round of selection, the % of the total input RNA that was specifically eluted from the resin by 5 mM target free in solution is plotted. After 4 rounds of selection, both pools showed enrichment for binding to caffeine. After 5 rounds of selection approximately 40% of each pool bound the caffeine resin and was specifically eluted by 5 mM caffeine in selection buffer. After 5 rounds of binding selection, a significant portion of the pool bound to and was specifically eluted from the caffeine resin.

The pool template resulting from round 4 of the selection using the HH33AG pool was amplified by PCR using the primers STC.12.143.A and STC.12.143.B to replace the active site G68 with the wild type A68 residue, restoring the potential for catalytic activity in those pool molecules. This template and the template emerging from round 4 of the HH33WT selection were used to generate pool RNAs for activity-based selections. The transcription conditions were exactly as described for the binding-based portion of the selection.

In total, two caffeine binding pools moved forward into activity-based selection.

B. Aspartame Binding Selections

This selection involved enrichment of the pool for molecules with the hammerhead motif that bind to aspartame, followed by selection for activation of cleavage activity by aspartame. Two pools, HH₃₃WT and HH₃₃AG, were used.

The binding-based portion of the selection carried out using the following procedure. Aspartame resin slurry was added to a disposable 1 cm column and allowed to settle to a bed volume of approximately 300 μL. The resin was washed 3 times with 500 μl of selection buffer (150 mM NaCl, 10 mM MgCl₂, 1 mM EDTA, 10 mM sodium phosphate, 90 mM Hepes, pH 7.1) then washed 3 times with 5 ml of selection buffer plus 10 μg/ml tRNA. Pool RNA (20 μM in round 1, ˜2 μM in subsequent rounds) in 300 μL selection buffer was added to the resin and incubated for 5 minutes. The resin was then washed approximately 12 times with 300 μL of selection buffer. Each wash fraction was incubated on the resin for 3 minutes. The resin was next treated with 6-7 300 μL washes with selection buffer containing 5 mM aspartame to elute any RNA molecules bound to the immobilized aspartame. All fractions were quantitated using the Bioscan QC 2000 counter. In rounds subsequent to round 1, the resin and sample volumes were reduced to 200 μL and the pool RNA was first passed through a pre-column composed of cysteine coupled to thiopropyl Sepharose resin to remove matrix binding RNAs. The elution fractions were combined and 300 mM sodium acetate, 50 mM EDTA, 40 μg glycogen and 1 volume of isopropanol were added.

Reverse transcription was carried out essentially as prescribed by the Invitrogen ThermoScript RT-PCR™ system instructions. The RNA incubated with an excess of primer STC.12.143.B 5′-AAAGGGCAACCTACGGCTTTCACCGTTTC-3′ (SEQ ID NO:20) for the HH₃₃WT pool or STC.12.143.C₅′-AAAGGGCAACCCACGGCTTTCACCGTTTC-3′ (SEQ ID NO:79) for the HH₃₃AG pool and 1.6 mM each dNTP at 65° C. for 5 minutes followed by rapid cooling to 4° C. Reverse transcriptase and buffer components were added and the mixture was incubated for 30 minutes at 50° C. (50 mM Tris acetate pH 8.4, 75 mM KOAc, 8 mM Mg(OAc)₂, ˜1 mM dNTP, 5 mM DTT, 2 units/μL RNaseOUT™, 0.75 units/mL ThermoScript RT, unidentified stabilizer). The RT mixture was added directly to a PCR reaction mix (20 mM Tris pH 8.4, 50 mM KCl, 2 mM MgCl₂, 0.5 μM STC.12.143.A, 0.5 μM STC.12.143.B (or STC.12.143.C), 0.5 mM each dNTP, 0.05 units/μL taq polymerase) and amplified by PCR (94° C. 1 min, 55. C 11 min, 72° C. 1 min; in later rounds, the incubation times were reduced to 30 seconds). The PCR product was purified using a QIAquick PCR purification kit (Qiagen), following the manufacturer's instructions. Approximately half of the DNA template was used for transcription of pool RNA for the subsequent round. Transcription pool RNA was carried out overnight at 37° C. (0.75 μM template, 40 mM Tris pH 7.8, 25 mM MgCl₂, 1 mM spermidine, 0.1% triton x-100, 5 mM each NTP, 40 mM DTT, ˜1 units/mL T7 RNA polymerase, 10 μCi α-³²P UTP). The transcriptions were quenched with 50 mM EDTA, ethanol precipitated then purified on 1.5 mm denaturing polyacrylamide gels (8M urea, 10% acrylamide; 19:1 acrylamide:bisacrylamide). Full length product RNA was removed from the gel by electroelution in an Elutrap® apparatus at 225V for 1 hour in 1×TBE. The eluted material was precipitated by the addition of 300 mM sodium acetate and 1 volume isopropanol.

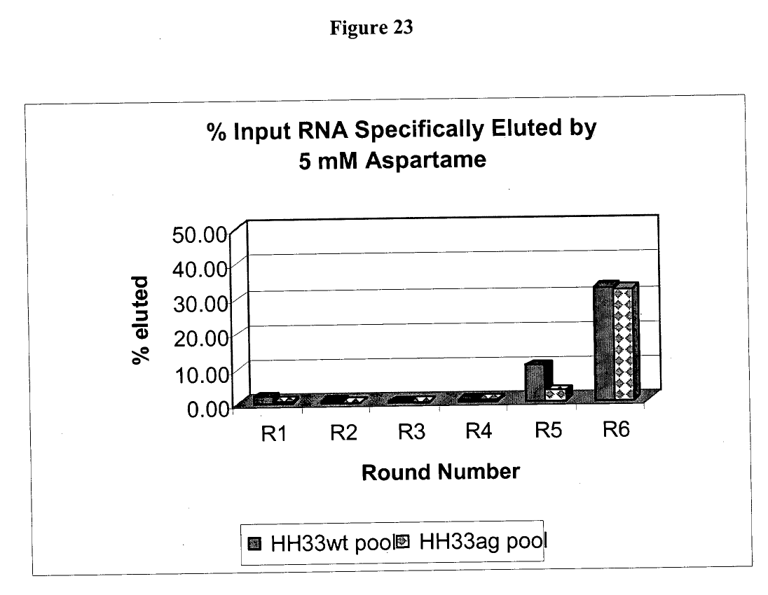

As shown in FIG. 23, the pool was enriched for binding to the target after several rounds of selection. The percent of pool RNA specifically eluted from aspartame derivatized resin is plotted vs. round of selection. For each round of selection, the % of the total input RNA that was specifically eluted from the resin by 5 mM target free in solution is plotted. After 5 rounds of selection, both pools showed enrichment for binding to aspartame. After 6 rounds of selection approximately 30% of each pool bound the aspartame resin and was specifically eluted by 5 mM aspartame in selection buffer.

The pool template resulting from round 5 of the selection using the HH₃₃AG pool was amplified by PCR using the primers STC.12.143.A and STC.12.143.B to replace the active site G68 with the wild type A68 residue, restoring the potential for catalytic activity in those pool molecules. This template and the template emerging from round 5 of the HH₃₃WT selection were used to generate pool RNAs for activity-based selections. The transcription conditions were exactly as described for the binding-based portion of the selection.

To increase the diversity of the aspartame binding pools, mutagenic PCR was performed on the templates emerging from round 5. PCR reactions were set up using pool template (˜200 ng per 100 μl) in a PCR reaction mix consisting of the following: 10 mM Tris pH 8.3, 50 mM KCl, 7 mM MgCl₂, 2 μM STC.12.143.A, 2 μM STC.12.143.B, 1 mM dCTP, 1 mM dTTP, 0.2 mM dATP, 0.2 mM dGTP, 0.5 mM MnCl₂, 0.05 units/μL taq polymerase. A series of amplifications and dilutions was performed, such that a minimum of 1.7 doublings per PCR cycle was achieved (94° C. 30 sec, 55° C. 30 sec, 72° C. 1 min). After four PCR cycles, 10% of the reaction mix was transferred to a fresh PCR reaction mix. Additional MnCl₂ and taq polymerase were added to the new reaction, and continued for four more cycles. After 324-cycle amplifications and 10% dilutions, the pool templates were purified using a QIAquick PCR purification kit (Qiagen), following the manufacturer's instructions. These mutagenized templates were used to generate pool RNAs for activity-based selections. The transcription conditions were exactly as described for the binding-based portion of the selection.

In total, two aspartame binding pools moved forward into activity-based selection.

II. Activity Selections

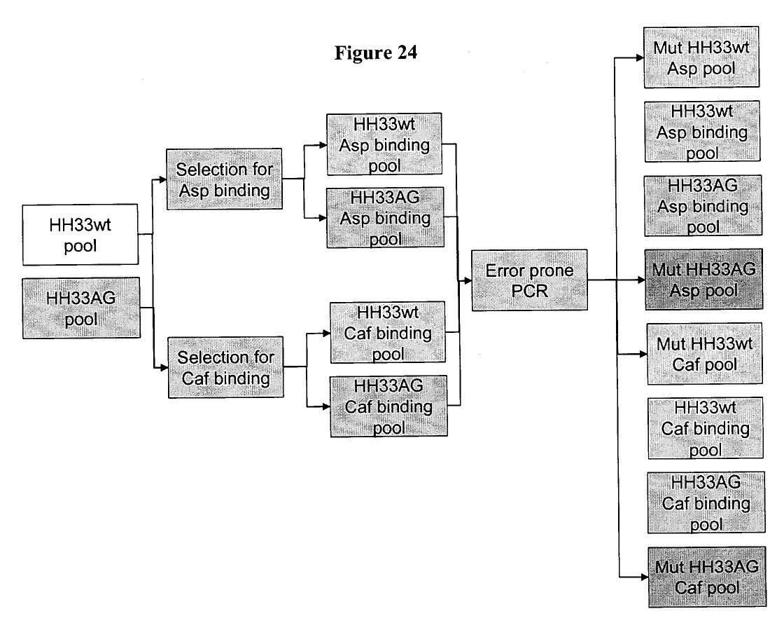

Two experimental tracks were pursued for the caffeine- and aspartame-binding pools which entered the activity-based portion of the selection protocol. In the first, the pools were being subjected to activity-based selection directly, i.e. without any modification. In the second track, the pools were mutagenized approximately 10% using error prone PCR prior to initiating activity-based selection. This reintroduced a limited amount of diversity to the binding pool to increase the chance that a sequence with the potential for target-dependent activation is present in the pool. The pools carried forward into activity-based selection are shown in FIG. 24.

A. Caffeine Activity Selections

The activity-based selections were carried out essentially as described for strategy 2. Pool RNA (˜10 μM) was incubated in selection buffer (500 mM NaCl, 10 mM MgCl₂, 1 mM EDTA, 10 mM sodium phosphate, 90 mM Hepes, pH 7.5) at room temperature for a fixed period. The reaction was quenched by the addition of 25 mM EDTA, 40 μg glycogen and 1 volume isopropanol. After precipitation, the RNA pellet was resuspended in 40% formamide, 2.5. mM EDTA, heated at 90° C. for 3 minutes then loaded onto a 10% denaturing acrylamide gel (1.5 mm). The band representing full length pool RNA was removed from the gel by electroelution in an Elutrap®. apparatus at 225V for 1 hour in 1×TBE. The eluted material was precipitated by the addition of 300 mM sodium acetate and 1 volume isopropanol. For the positive selection step, the pool RNA carried from the negative step was incubated in selection buffer plus 5 mM caffeine for a fixed period. The reaction was quenched by the addition of 25 mM EDTA and 1 volume isopropanol. After precipitation, the RNA pellet was resuspended in 40% formamide, 2.5 mM EDTA, heated at 90° C. for 3 minutes then loaded onto a 10% denaturing acrylamide gel (1.5 mm). The band representing cleaved pool RNA was removed from the gel by electroelution in an Elutrap® apparatus at 225V for 1 hour in 1×TBE. The eluted material was precipitated by the addition of 300 mM sodium acetate and 1 volume isopropanol.

Reverse transcription was carried out essentially as prescribed by the Invitrogen ThermoScript RT-PCR™ system instructions. The RNA incubated with an excess of primer STC.12.143.B 5′-AAAGGGCAACCTACGGCTTTCACCGTTTC-3′ (SEQ ID NO: 20) and 1.6 mM each dNTP at 65° C. for 5 minutes followed by rapid cooling to 4 C. Reverse transcriptase and buffer components were added and the mixture was incubated for 30 minutes at 50° C. (50 mM Tris acetate pH 8.4, 75 mM KOAc, 8 mM Mg(OAc)₂, ˜1 mM dNTP, 5 mM DTT, 2 units/mL RNaseOUT™, 0.75 units/μL ThermoScript RT, unidentified stabilizer). The RT mixture was added directly to a PCR reaction mix (20 mM Tris pH 8.4, 50 mM KCl, 2 mM MgCl₂, 0.5 μM STC.12.143.A, 0.5 μM STC.12.143.B, 0.5 mM each dNTP, 0.05 units/μL taq polymerase) and amplified by PCR (94° C. 1 min, 55° C. 1 min, 72° C. 1 min; in later rounds, the incubation times were reduced to 30 seconds). The PCR product was purified using a QIAquick PCR purification kit (Qiagen), following the manufacturer's instructions. Approximately half of the DNA template was used for transcription of pool RNA for the subsequent round. Transcription was carried out overnight at 37° C. (0.75 μM template, 40 mM Tris pH 7.8, 25 mM MgCl₂, 1 mM spermidine, 0.1% triton x-100, 5 mM each NTP, 40 mM DTT, ˜1 units/μL T7 RNA polymerase, 10 μCi α-³²P UTP). The transcriptions were quenched with 50 mM EDTA, ethanol precipitated then purified on 1.5 mm denaturing polyacrylamide gels (8M urea, 10% acrylamide; 19:1 acrylamide:bisacrylamide). Full length product RNA was removed from the gel by electroelution in an Elutrap® apparatus at 225V for 1 hour in 1×TBE. The eluted material was precipitated by the addition of 300 mM sodium acetate and 1 volume isopropanol.

Beginning in round four, the protocol was modified such that the pool RNA would undergo three cycles of denaturation and refolding during each negative selection step. Briefly, pool RNA (10 μM) was incubated in selection buffer at room temperature for a fixed period. The reaction was quenched by the addition of 25 mM EDTA, the mixture was heated at 90° C. for 1 minute then cooled at room temperature for 1 minute followed by the addition of one volume of isopropanol. The pellet was resuspended in selection buffer and the above protocol was repeated such that in total the pool RNA was subjected to three incubations in selection buffer and two or three denaturation steps during each negative selection step. The positive selection steps were conducted as described above.

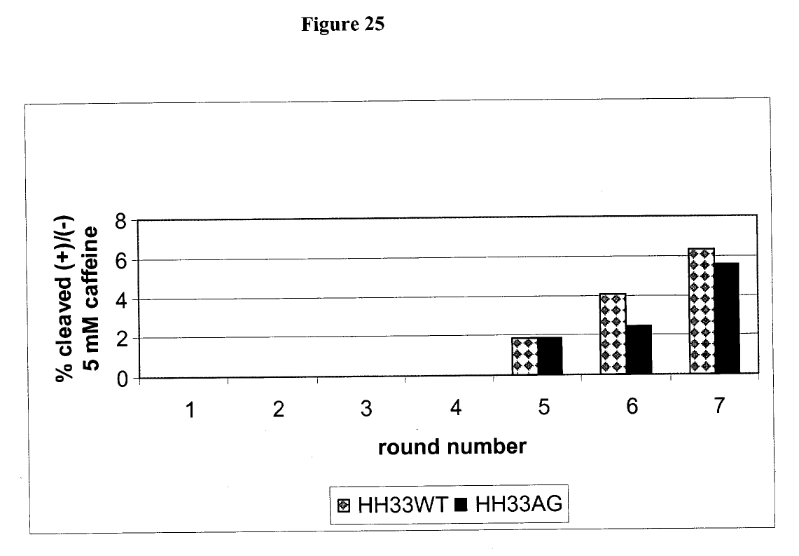

Beginning in round 5, the activity of the post-negative step pool RNA in the presence and absence of 5 mM caffeine was measured during each round. The RNA pellet resulting from the negative selection step was resuspended in 145 μL selection buffer. 125 μL of the solution was carried forward into the positive selection. The remaining 20 μL was divided quickly into two 10 μL aliquots. To one aliquot, 10 μL selection buffer was added and to the other was added 10 μL selection buffer plus 5 mM caffeine. These reaction mixtures were incubated for a fixed period, then quenched with 25 mM EDTA and precipitated with one volume of isopropanol. After precipitation, the RNA pellet was resuspended in 40% formamide, 2.5 mM EDTA, heated at 90° C. for 3 minutes then loaded onto a 10% denaturing acrylamide gel (1.5 mm). The extent of cleavage in the two reactions was quantitated using a Storm Phosphoimager (Molecular Dynamics). The progress of the selection was monitored by calculating the ratio of the extent of cleavage in the presence of 5 mM caffeine vs. the extent of cleavage in the absence of caffeine. When the pool exhibited caffeine-dependent activity, this ratio exceeds one. The plot in FIG. 25 indicates that between selection rounds 5 and 7 significant caffeine-dependent activity emerged in both pools.

Following round 7, the pool templates were cloned using the TOPO TA cloning kit (Invitrogen) following the manufacturer's instructions. 24 colonies were isolated and the inserts were amplified by PCR (20 mM Tris pH 8.4, 50 mM KCl, 2 mM MgCl₂, 0.5 μM STC.12.143.A, 0.5 μM STC.12.143.B, 0.5 mM each dNTP, 0.05 units/μL taq polymerase). The resulting template DNA's were purified using a QIAquick PCR purification kit. Template DNA was used to program run-off transcription (˜0.75 μM template, 40 mM Tris pH 7.8, 25 mM MgCl₂, 1 mM spermidine, 0.1% triton x-100, 5 mM each NTP, 40 mM DTT, ˜1 units/μL T7 RNA polymerase, 5 μCi α-³²P UTP) mixtures, incubated overnight at 37° C. The transcriptions were quenched with 50 mM EDTA, ethanol precipitated then purified on 1.5 mm denaturing polyacrylamide gels (8M urea, 10% acrylamide; 19:1 acrylamide:bisacrylamide). Full length product RNA was removed from the gel by electroelution in an Elutrap® apparatus at 225V for 1 hour in 1×TBE. The eluted material was precipitated by the addition of 300 mM sodium acetate and 1 volume isopropanol. The sequences of Caffeine sensors are presented in Table 5.

TABLE 5

Sequences for caffeine sensors.

| | | |
|---|---|---|
| | | SEQ |
| | | ID |
| Identifier | Sequence | NO |

HHwt

| | | |
|---|---|---|
| ARX12P1.A03 | GGGCGACCCTGATGAGGATGGTGCAGCNTACTCCGCCAGGCGATCGACCCGAAACG | 80 |
| | GTGAAAGCCGTAGGTTGCCCTTT | |
| ARX12P1.C01 | GGGCGACCCTGATGAGGNNCNTGTGTACATNTNCTACCCNNACCGATTACGAAACG | 81 |
| | GTGAAAGCCGTAGGTTGCCCTTT | |
| ARX12P1.G02 | GGGCGACCCTGATGAGGTCCCGCCATCACACCTATTGCTGCTGACATTGCGAAACG | 82 |
| | GTGAAAGCCGTAGGTTGCCCTTT | |
| ARX12P1.G04 | GGGCGACCCTGATGAGACGGTAATGTTCGGCTAGTTCTCAAACACTCCTCGAAACG | 83 |
| | GTGAAAGCCGTAGGTTGCCCTTT | |
| ARX12P1.A01 | GGGCGACCCTGATGAGGATAGATCGTACTGCCAGATGTGATTGCCTGGCCGAAACG | 84 |
| | GTGAAAGCCGTAGGTTGCCCTTT | |
| ARX12P1.D06 | GGGCGACCCTGATGAGCGCAAACTTAAGTCAGAAGACAGTCATCCTGCCGAAACGG | 85 |
| | TGAAAGCCGTGGTTGCCCTTT | |
| ARX12P1.G09 | GGGCGACCCTGATGAGGACCGGAGTGATGCCCGGATACCAACGCATCCCCGAAACG | 86 |
| | GTGAAAGCCGTAGGTT | |

Hhga

| | | |
|---|---|---|
| ARX12P1.G06 | GGGCGACCCTGATGAGATAGCCTTCGTAGACATCAGAACCCGTTGGTAGCGAAACG | 87 |
| | GTGAAAGCCGTAGGTTGCCCTTT | |
| ARX12P1.D07 | GGGCGACCCTGATGAGTCNTATGTAGCCTCTGTATGGCGCGTTATNCANCGAAACG | 88 |
| | GTGAAAGCCGTAGGTTGCCCTTT | |
| ARX12P1.C08 | GGGCGACCCTGATGAGCGCAGAGGAAGCGAGCTCTTACTGAGTCACTGTCGAAACG | 89 |
| | GTGAAAGCCGTAGGTTGCCCTTT | |
| ARX12P1.H08 | GGGCGACCCTGATGAGGGTGCACGACTCTACATCTGGAACGATCTCAAGCGAAACG | 90 |
| | GTGAAAGCCGTAGGTTGCCCTTT | |
| ARX12P1.G05 | GGGCGACCCTGATGAGGGACAGGAAGTCGGCGCTCCCCAGCGTGTGTCACGAAACG | 91 |
| | GTGAAAGCCGTAGGTTGCCCTTT | |
| ARX12P1.H06 | GGGCGACCCTGATGAGACGATGGAAGTCGGCATTACACAATGTGATCGGCGAAACG | 92 |
| | GTGAAAGCCGTAGGTTGCCCTTT | |
| ARX12P1.E08 | GGGCGACCCTGATGAGCGCAGAGGAAGCGAGCTCTTACTGAGTCACTGTCGAAACG | 93 |
| | GTGAAAGCCGTAGGTTGCCCTTT | |

The activity of the individual clones was measured by incubating the RNA (1 μM) in selection buffer in the presence or absence of 5 mM caffeine at room temperature for 20 minutes. The reactions were quenched by the addition of 25 mM EDTA and 1 volume isopropanol. After precipitation, the RNA pellet was resuspended in 40% formamide, 2.5 mM EDTA, heated at 90° C. for 3 minutes then loaded onto a 10% denaturing acrylamide gel (1.5 mm). The extent of cleavage in the two reactions was quantitated using a Storm Phosphoimager (Molecular Dynamics). The results are summarized in Table 6. The switch factor is defined as the percent cleavage observed in the presence of 5 mM caffeine divided by the percent cleavage observed in the absence of caffeine.

TABLE 6
Caffeine sensor activity summary.

| Clone identifier | Clone identifier | |
|---|---|---|
| (LARK) | (internal) | Switch Factor^{a} |
| | AF.35.111.A1 | 6.1 |
| ARX12P2.D06 | AF.35.111.A2 | 33.8 |
| | AF.35.111.A3 | 7.3 |
| | AF.35.111.A4 | 6.2 |
| | AF.35.111.A5 | 1 |
| | AF.35.111.A6 | 37.8 |
| | AF.35.111.A7 | 8.8 |
| ARX12P2.E08 | AF.35.111.A8 | 5.5 |
| | AF.35.111.A9 | 5.0 |
| | AF.35.111.A10 | 3.5 |
| | AF.35.111.A11 | 1.4 |
| | AF.35.111.A12 | 6.1 |
| | AF.35.111.B1 | .9 |
| ARX12P2.G09 | AF.35.111.B2 | 7.6 |
| | AF.35.111.B3 | 7.7 |
| | AF.35.111.B4 | 7.9 |
| | AF.35.111.B5 | 13.6 |
| | AF.34.111.B6 | 11.3 |
| | AF.34.111.B7 | 6.0 |
| | AF.34.111.B8 | 6.3 |
| | AF.34.111.B9 | 1.1 |
| | AF.34.111.B10 | 7.6 |
| | AF.34.111.B11 | 5.3 |
| | AF.34.111.B12 | 1.0 |

^{a}Single time point 20 min assay using a gel-based readout; 1 × selection buffer, 5 mM caffeine

B. Aspartame Activity Selections

The activity-based selection was carried out using the following procedure. Pool RNA (˜10 μM) was incubated in selection buffer (500 mM NaCl, 10 mM MgCl₂, 1 mM EDTA, 10 mM sodium phosphate, 90 mM Hepes, pH 7.5) at room temperature for a fixed period. The reaction was quenched by the addition of 25 mM EDTA, 300 mM NaOAc, 40 μg glycogen and 1 volume isopropanol. After precipitation, the RNA pellet was resuspended in 40% formamide, 2.5 mM EDTA, heated at 90° C. for 3 minutes then loaded onto a 10% denaturing acrylamide gel (1.5 mm). The band representing full length pool RNA was removed from the gel by electroelution in an Elutrap® apparatus at 225V for 1 hour in 1×TBE. The eluted material was precipitated by the addition of 300 mM sodium acetate and 1 volume isopropanol. For the positive selection step, the pool RNA carried from the negative step was incubated in selection buffer plus 5 mM aspartame for a fixed period. The reaction was quenched by the addition of 25 mM EDTA, 300 mM NaOAc and 1 volume isopropanol. After precipitation, the RNA pellet was resuspended in 40% formamide, 2.5 mM EDTA, heated at 90° C. for 3 minutes then loaded onto a 10% denaturing acrylamide gel (1.5 mm). The band representing cleaved pool RNA was removed from the gel by electroelution in an Elutrap® apparatus at 225V for 1 hour in 1×TBE. The eluted material was precipitated by the addition of 300 mM sodium acetate and 1 volume isopropanol.

Reverse transcription was carried out essentially as prescribed by the Invitrogen ThermoScript RT-PCR™ system instructions. The RNA incubated with an excess of primer STC.12.143.B 5′-AAAGGGCAACCTACGGCTTTCACCGTTTC-3′ SEQ ID NO:20) and 1.6 mM each dNTP at 65° C. for 5 minutes followed by rapid cooling to 4° C. Reverse transcriptase and buffer components were added and the mixture was incubated for 30 minutes at 50° C. (50 mM Tris acetate pH 8.4, 75 mM KOAc, 8 mM Mg(OAc)₂, ˜1 mM dNTP, 5 mM DTT, 2 units/mL RNaseOUT™, 0.75 units/μL ThermoScript RT, unidentified stabilizer). The RT mixture was added directly to a PCR reaction mix (20 mM Tris pH 8.4, 50 mM KCl, 2 mM MgCl₂, 0.5 mM STC.12.143.A, 0.5 mM STC.12.143.B, 0.5 mM each dNTP, 0.05 units/mL taq polymerase) and amplified by PCR (95. ° C. 1 min, 55° C. 1 min, 72° C. 1 min; in later rounds, the incubation times were reduced to 30 seconds). The PCR product was purified using a QIAquick PCR purification kit (Qiagen), following the manufacturer's instructions. Approximately one quarter of the DNA template was used for transcription of pool RNA for the subsequent round. Transcription was carried out overnight at 37° C. (0.75 μM template, 40 mM Tris pH 7.8, 25 mM MgCl₂, 1 mM spermidine, 0.1% triton x-100, 5 mM each NTP, 40 mM DTT, ˜1 units/μL T7 RNA polymerase, 10 μCi α-³²P UTP). The transcriptions were quenched with 50 mM EDTA, ethanol precipitated then purified on 1.5 mm denaturing polyacrylamide gels (8M urea, 10% acrylamide; 19:1 acrylamide:bisacrylamide). Full length product RNA was removed from the gel by electroelution in an Elutrap®. apparatus at 225V for 1 hour in 1×TBE. The eluted material was precipitated by the addition of 300 mM sodium acetate and 1 volume isopropanol.

Beginning in round four, the protocol was modified such that the pool RNA would undergo three cycles of denaturation and refolding during each negative selection step. Briefly, pool RNA (˜10 μM) was incubated in selection buffer at room temperature for a fixed period. The reaction was quenched by the addition of 25 mM EDTA and 300 mM NaOAc. The mixture was heated at 90° C. for 1 minute then cooled at room temperature for 1 minute followed by the addition of one volume of isopropanol. The pellet was resuspended in selection buffer and the above protocol was repeated such that in total the pool RNA was subjected to three incubations in selection buffer and two or three denaturation steps during each negative selection step. The positive selection steps were conducted as described above.

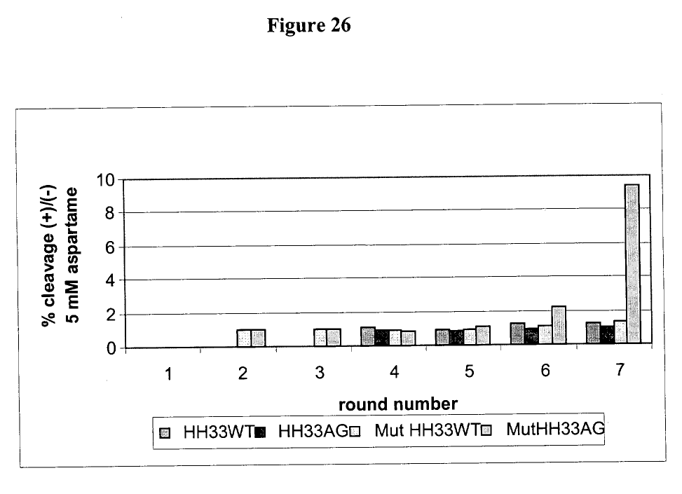

Beginning in round 2, the activity of the post-negative step pool RNA in the presence and absence of 5 mM aspartame was measured during each round. The RNA pellet resulting from the negative selection step was resuspended in 145 μL selection buffer. 125 μL of the solution was carried forward into the positive selection. The remaining 20 μL was divided quickly into two 10 μL aliquots. To one aliquot, 10 μL selection buffer was added and to the other was added 10 μL selection buffer plus 5 mM aspartame. These reaction mixtures were incubated for a fixed period, then quenched with 25 mM EDTA, 300 mM NaOAc and precipitated with one volume of isopropanol. After precipitation, the RNA pellet was resuspended in 40% formamide, 2.5 mM EDTA, heated at 90° C. for 3 minutes then loaded onto a 10% denaturing acrylamide gel (1.5 mm). The extent of cleavage in the two reactions was quantitated using a Storm Phosphoimager (Molecular Dynamics). The progress of the selection was monitored by calculating the ratio of the extent of cleavage in the presence of 5 mM aspartame vs. the extent of cleavage in the absence of aspartame. When the pool exhibits aspartame-dependent activity, this ratio exceeds one. The plot in FIG. 26 shows that between selection rounds 5 and 7 significant aspartame-dependent activity emerged in the mutHH₃₃AG pool.

Following round 7, the pool templates were cloned using the TOPO TA cloning kit (Invitrogen) following the manufacturer's instructions. 24 colonies were isolated and the inserts were amplified by PCR (20 mM Tris pH 8.4, 50 mM KCl, 2 mM MgCl₂, 0.5 μM STC.12.143.A, 0.5 μM STC.12.143.B, 0.5 mM each dNTP, 0.05 units/μL taq polymerase). The resulting template DNA's were purified using a QIAquick PCR purification kit. Template DNA was used to program run-off transcription (˜0.75 μM template, 40 mM Tris pH 7.8, 25 mM MgCl₂, 1 mM spermidine, 0.1% triton x-100, 5 mM each NTP, 40 mM DTT, ˜1 units/μL T7 RNA polymerase, 5 α-³²P UTP) mixtures, incubated overnight at 37° C. The transcriptions were quenched with 50 mM EDTA, ethanol precipitated then purified on 1.5 mm denaturing polyacrylamide gels (8M urea, 10% acrylamide; 19:1 acrylamide:bisacrylamide). Full length product RNA was removed from the gel by electroelution in an Elutrap® apparatus at 225V. for 1 hour in 1×TBE. The eluted material was precipitated by the addition of 300 mM sodium acetate and 1 volume isopropanol. R8 HHga(mut) aspartame sensor clone sequences are shown in Table 7, 6 of the clones had the same sequence.

TABLE 7

Aspartame clone sequences.

| | | |
|---|---|---|
| | | SEQ |
| | | ID |
| Identifier | Sequence | NO |

| | | |
|---|---|---|
| ARX15P1.A02 | GGGCGACCCTGATGAGCAGGCAAACGTGCGCCTAGAATGCAGACACCAACGAAACGGTG | 94 |
| | AAAGCCGTAGGTTGCCCTTT | |
| ARX15P1.A03 | GGGCGACCCTGATGAGCAGGCAAACGTGCGCCTAGAATGCAGACACCAACGAAACGGTG | 95 |
| | AAAGCCGTAGGTTGCCCTTT | |
| ARX15P1.B04 | GGGCGACCCTGATGAGCAGGCAAACGTGCGCCTAGAATGCAGACACCAACGAAACGGTG | 96 |
| | AAAGCCGTAGGTTGCCCTTT | |
| ARX15P1.B06 | GGGCGACCCTGATGAGCAGGCAAACGTGCGCCTAGAATGCAGACACCAACGAAACGGTG | 97 |
| | AAAGCCGTAGGTTGCCCTTT | |
| ARX15P1.H04 | GGGCGACCCTGATGAGCAGGCAAACGTGCGCCTAGAATGCAGACACCAACGAAACGGTG | 98 |
| | AAAGCCGTAGGTTGCCCTTT | |
| ARX15P1.E06 | GGGCGACCCTGATGAGCAGGCAAACGTGCGCCTAGAATGCAGACACCAACGAAACGGTG | 99 |
| | AAAGCCGTAGGTTGCCCTTT | |
| ARX15P1.G03 | GGGCGACCCTGATGAGCAGGCAAACGTGCGCCTAGAATGCAGACACCAACGAAACGGTG | 100 |
| | AAAGCCGTAGGTTGCCCTTT | |
| ARX15P1.E05 | GGGCGACCCTGATGAGCAGGCAAACGTGCGCCTAGAATGCAGACACCAACGAAACGGTG | 101 |
| | AAAGCCGTAGGTTGCCCTTT | |
| ARX15P1.H02 | GGGCGACCCTGATGAGCAGGCAAACGTGCGCCTAGAATGCAGACACCAACGAAACGGTG | 102 |
| | AAAGCCGTAGGTTGCCCTTT | |
| ARX15P1.C03 | GGGCGACCCTGATGAGCAGGCAAACGTGCGCCTAGAATGCAGGCACCAACGAAACGGTG | 103 |
| | AAAGCCGTAGGTTGCCCTTT | |
| ARX15P1.H06 | GGGCGACCCTGATGAGTGGTGCAAGTTAGTCGCGTCCTTAGTCGCTACACGAAACGGTG | 104 |
| | AAAGCCA | |
| ARX15P1.A06 | GGGCGACCCTGATGAGTGGTGCAAGTTAGTCGCGNNCTTAGNNGCTACACGAAACGGTG | 105 |
| | AAAGCCGTAGGTTGCCCTTT | |
| ARX15P1.A05 | GGGCGACCCTGATGAGCAGCTACTCGTGCACGAGAGTTTCGTTGAAGTGCGAAACGGTG | 106 |
| | AAAGCCGTAGGTTGCCCTTT | |
| ARX15P1.F06 | GGGCGACCCTGATGAGCATCTACTCGCGCGCGAGAGTTTCGTTGAAGTGCGAAACGGTG | 107 |
| | AAAGCCGTAGGTTCCCTTT | |
| ARX15P1.D04 | GGTCGACCCTGATGAGGGAGCGAGTTAGTTTGCCATCGGTCGTGCNGCCNCCGAAACGG | 108 |
| | TGAAANCCGTAGGTTGCCCTTT | |
| ARX15P1.E04 | GGGCGACCCTGATGAGCGGTGCTAGTTAGTTGCAGTTTCGGTTGTTACGCGAAACGGTG | 109 |
| | AAAGCCGTAGGTTGCCCTTT | |
| ARX15P1.C02 | GGGCGACCCTGATGAGCGGTNCTAGTTAGTTGCAGTTTCGTCTGTTACGCGAAACGGTG | 110 |
| | AAAGCCGTAGGTTGCCCTTT | |
| ARX15P1.C01 | GGGCGACCCTGATGAGCGGTGCTAGTTAGTTGCGGTTTAGGCTGTTACGCGAAACGGTG | 111 |
| | AAAGCCGTAGGTTGCCCTTT | |
| ARX15P1.F04 | GGGCGACCCTGATGAGGGAGCGAGTTAGTTGCCATCGGCGTGTGGCTACCGAAACGGTG | 112 |
| | AAAGCCGTAGGTTGCCCTTT | |
| ARX15P1.B01 | GGGCGACCCTGATGAGCCCACGCTAGTCAGTTACATTGCCCCTACGACGAAACGGTGAAA | 113 |
| | AGCCGTAGGTTGCCCTTT | |
| ARX15P1.B05 | GGGCGACCCTGATGAGTCGGGCAATTCGAATGACATGCGTGTTGAGACACGAAACGGTG | 114 |
| | AAAGCCGTAGGTTGCCCTTT | |
| ARX15P1.D02 | GGGCGACCCTGATGAGTCCGTTAGAGCCGGAAGACGTAAAACTCGCCGAAACGGTGAAA | 115 |
| | GCCGTAGGTTGCCCTTT | |
| ARX15P1.G06 | GGGCGACCCTGATGAGCGGTGCTAGTTAGTAGCAGATTTGGCTGCTACGCGAAACGGTG | 116 |
| | AAAGCCGTAGGTTGCCCTTT | |
| ARX15P1.G02 | GGGCGACCCTGATGAGTGCGTNCTCGCTACNAGCACCTTANAAGGTTCACGAAACGGTG | 117 |
| | AAAGCCGTAGGTTGCCCTTT | |
| ARX15P1.A01 | GGGCGACCCTGATGAGTTGGGCAATTAGAATGACATGCGTGCTGAGACCCGAAACGGTG | 118 |
| | AAAGCCGTAGGTTGCCCTTT | |
| ARX15P1.G04 | GGGCGACCCTGATGAGAACAAGCAGGAGTCTTTCCGGGCGCTCCGAGGACGAAACGGTG | 119 |
| | AAAGCCGTAGGTTGCCCTTT | |
| ARX15P1.D03 | GGGCGACCCTGATGAGGAACTAGCGCGTCCTACTGTCGAACATGTGCCCCGAAACGGTG | 120 |
| | AAAGCCGTAGGTTGCCCTTT | |
| ARX15P1.B03 | GGGCGACCCTGATGAGCATCTCTTAGAAGAGAGCAGGGATACTTCTCGCGAAACGGTGA | 121 |
| | AAGCCGTAGGTTGCCCTTT | |
| ARX15P1.G05 | GGGCGACCCTGATGAGCAGGAAAAGGAAAGCGTTCATCGCTCACACCAACGAAACGGTG | 122 |
| | AAAGCCGTAGGTTGCCCTTT | |
| ARX15P1.E02 | GGGCGACCCTGATGAGGGAGCCGCAATTCACGGTATAAGAATCTGCCCACGAAACGGTG | 123 |
| | AAAGCCGTAGGTTGCCCTTT | |
| ARX15P1.C05 | GGGCGACCCTGATGAGGACGTTAGAGCCGTCGTCAAAAACTTACCCGCCGAAACGGTGA | 124 |
| | AAGCCGTAGGTTGCCCTTT | |
| ARX15P1.H03 | GGGCGACCCTGATGAGCCGGCTTAGAAGCCCTAAGGGATACTTCCTACGCGAAACGGTG | 125 |
| | AAAGCCGTAGGTTGCCCTTT | |
| ARX15P1.F01 | GGCNGCCCTTAATNAGANNGTTACANNCGTCNTCAANNANTNCCCCTCCGAAACGGTGA | 126 |
| | AAGCNNTAGGTTGCCCTTT | |
| ARX15P1.H01 | GGGCGACCCTGATGAGTCGGGCAATTAGAATGACATGCGTGTCNAGNNNCNNAACGGTG | 127 |
| | AAANNNNTAGGTTGCCCTTT | |

The activity of the individual clones was measured by incubating the RNA (˜1 μM) in selection buffer in the presence or absence of 5 mM aspartame at room temperature for 20 minutes. The reactions were quenched by the addition of 25 mM EDTA and 1 volume isopropanol. After precipitation, the RNA pellet was resuspended in 40% formamide, 2.5 mM EDTA, heated at 90° C. for 3 minutes then loaded onto a 10% denaturing acrylamide gel (1.5 mm). The extent of cleavage in the two reactions was quantitated using a Storm Phosphoimager (Molecular Dynamics). The results are summarized in Table 8. The switch factor is defined as the percent cleavage observed in the presence of 5 mM aspartame divided by the percent cleavage observed in the absence of aspartame.

TABLE 8
Switch factors for selected aspartame sensors.

| Clone identifier (LARK) | Clone identifier (internal) | Switch Factor^{a} |
|---|---|---|
| | AF.35.147.A1 | 1.2 |
| ARX15P1.A02 | AF.35.147.A2^{b} | 55.9 |
| | AF.35.147.A3^{b} | 52.8 |
| ARX15P1.A06 | AF.35.147.A6 | 1.9 |
| ARX15P1.B01 | AF.35.147.B1 | 2.2 |
| | AF.35.147.B2 | 10.3 |
| | AF.35.147.B4^{b} | 42.1 |
| | AF.35.147.B5 | 2.8 |
| | AF.35.147.B6^{b} | 60.4 |
| ARX15P1.C03 | AF.35.147.C3 | 1.3 |
| ARX15P1.C05 | AF.35.147.C5 | 17.3 |
| ARX15P1.D02 | AF.35.147.D2 | 14.4 |
| ARX15P1.D03 | AF.35.147.D3 | 0.95 |
| | AF.35.147.E2 | 1.1 |
| ARX15P1.E04 | AF.35.147.E4 | 4.3 |
| | AF.35.147.E6^{b} | 48.6 |
| ARX15P1.F01 | AF.35.147.F1 | 25.2 |
| | AF.35.147.F5 | 49.1 |
| ARX15P1.G02 | AF.35.147.G2 | 9.6 |
| ARX15P1.G04 | AF.35.147.G4 | 0.95 |
| ARX15P1.G06 | AF.35.147.G6 | 1.3 |
| ARX15P1.H03 | AF.35.147.H3 | 2.8 |
| | AF.35.147.H4^{b} | 46.4 |
| ARX15P1.H01 | AF.35.147.H1 | 1 |

^{a}Measured in a single time point 20 minute assay using a gel-based readout; 1 × selection buffer, 5 mM aspartame.
^{b}These clones have the same sequence.

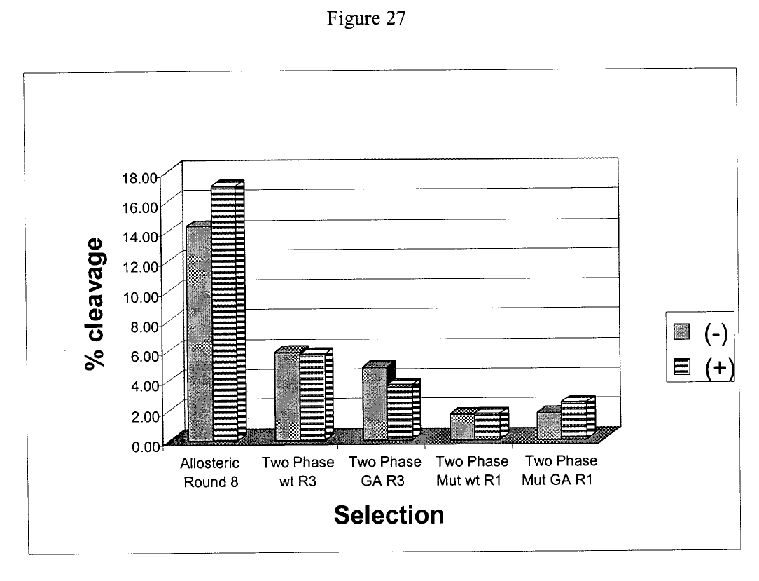

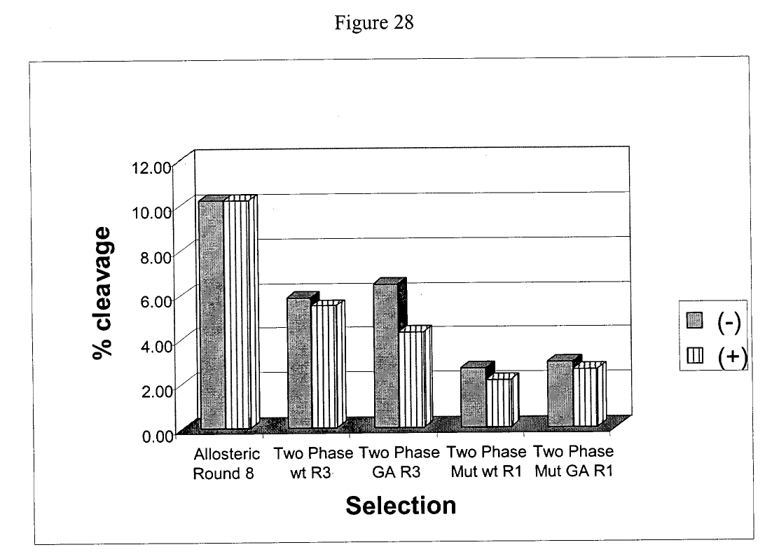

In sum, the caffeine pools went through 4 rounds of binding selection, then 7 rounds of allosteric selection. The aspartame pool went through 5 rounds of binding selection, followed by mutagenesis by PCR, then 7 rounds of allosteric selection. The final pool sequences were designated as R8. A summary of cleavage activity for the caffeine sensors is shown in FIG. 27. A summary of cleavage activity for the aspartame sensors is shown in FIG. 28. Cleavage was assayed in the presence of 5 mM target in a 45 minute reaction.

Three of the selections yielded a positive, target-dependent signal after several rounds of selection: AspMutHH33AG, CafHH33WT, and CafHH33AG. The two best clones, S3.caf.A2 and S3.caf.A6 were activated approximately 35-fold by caffeine. Sequence data for 48 clones emerging from the AspMutHH33GA pool revealed that a dominant sequence had emerged. The sequence, referred to as S3.asp.A2 is activated approximately 50-fold by 5 mM aspartame in a 20 minute assay, as analyzed by denaturing gel electrophoresis.

### Example 4

### General FRET Assays

Previously-described theophylline-, cGMP- and cAMP-dependent hammerhead constructs were used as models for the identification of a suitable hammerhead NASM FRET model system. Initial gel-based studies identified the cGMP construct as the sensor with the most favorable performance characteristics (rate and extent of cleavage, reliability etc.), hence this construct was chosen for the basic development of fluorescence resonance energy transfer (FRET) assays.

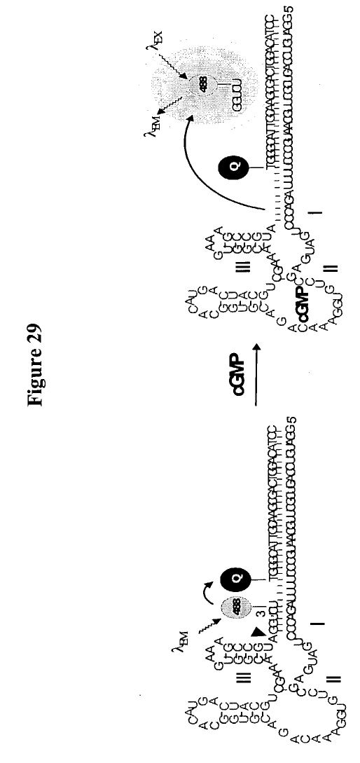

Various sequence derivatives of the original hammerhead construct were synthesized and, following periodate oxidation, 3′-labelled with fluorescein thiosemicarbazide (FAM). Hybridization with dabcyl, tamra or QSY-7 modified quencher oligonucleotides constituted a set of FRET NASM constructs with different spatial arrangements of dye/quencher combinations (See FIGS. 6-10). Analysis of the fluorescence signals in response to the addition of cGMP revealed the most suitable structure to be that shown in FIG. 29.

A typical protocol for periodate oxidation and fluorescein thiosemicarbazide labeling was as follows: For a 600 μL reaction, 300 μL sensor RNA (up to 30 nmoles in reaction), 150 μL 1.2 M NaOAc pH 5.4, 150 μL 40 mM stock NaIO₄ (sodium metaperiodate, 8.72 mg/ml) fresh stock made for every reaction were mixed and placed on ice, in the dark, for 1 hour. The reaction was then precipitated with 600 μL isopropyl alcohol and resuspended in 450 μL H₂O. The oxidized sensor RNA (450 μL) was then mixed with 150 μL 1.2 M NaOAc pH 5.4, 100 μL 40 mM stock fluorescein thiosemicarbazide (16.8 mg/ml) fresh stock in DMSO made for every reaction and the reaction was incubated at room temperature for two hours (or for a shorter time if the sensor underwent significant target-independent cleavage). The reaction was then precipitated with 700 μL isopropyl alcohol. The pellet was resuspended and gel purified on a gel containing 10% urea. The RNA was electroeluted, precipitated, resuspended and quantitated by measuring the absorbance at 260 mm⁻¹ (A₂₆₀). A 12 μM stock solution was prepared.

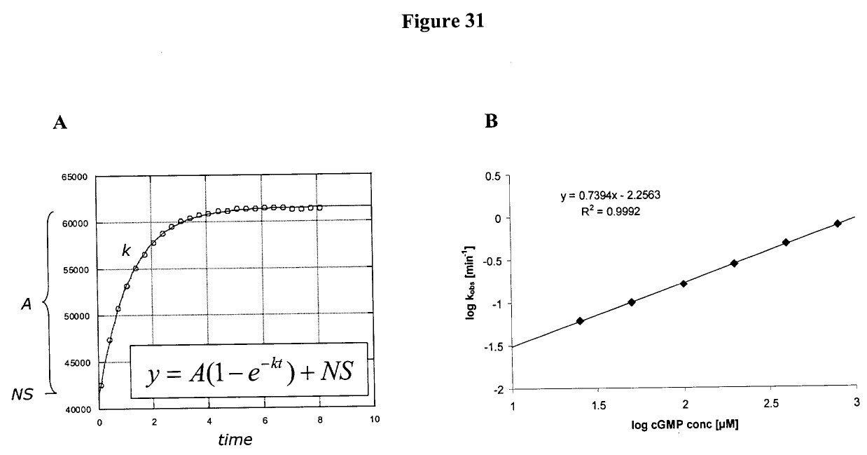

FRET experiments were monitored in real-time and sample volume, sensor concentrations, and assay setup were optimized. The data was analyzed through fitting the obtained time-course to a first order kinetic model. Target-dependence could be quantitated through correlating catalytic rate to cGMP concentration. Data showing the calculation of a cleavage rate constant for the cGMP activated NASM is shown in FIG. 31. See also FIG. 11 and discussion relating thereto.

1.2 μL of fluorescent sensor RNA (10 μM), 1.2 μM final concentration; 3 μL of 1 dabcyl quencher oligo (20 μM), 6 μM final concentration; 2.8 μL H₂O; and 2 μL 5×annealing buffer (150 mM Tris 7.4, 250 mM NaCl) were mixed and heated to 80° C. for 2 minutes, then cooled to room temperature. Then 1 μL of 10× hammerhead buffer (500 mM Tris.HCl pH 7.4, 200 mM MgCl₂) were added and the solution was equilibrated for 2 min. This reagent was stored at −20° C. or immediately used for assay. 1.2×cGMP target stocks were serially diluted in 1×Buffer (50 mM Tris.HCl pH 7.4, 20 mM MgCl₂).

The cleavage assays were done in Greiner black clear-bottom 96-well plates by adding 10 μL of above 6×quenched RNA to each well and monitoring sensor RNA stability in the fluorescein channel (Ex 495 Em 530) for 5 minutes. To start the reaction, 50 μL 1.2×target was added and mixed thoroughly. The Instrument settings (Packard Fusion) were as follows: Fluorescence top read:FAM EX 485 nm (20 nm bandpass) and FAM EM 535 (25 nM bandpass); Intensity: 10; Sample activity expected: Custom; Voltage: 900; Read time: 1s; Read each well: 1; Read each plate: 10-25; Delay between each plate: 0.

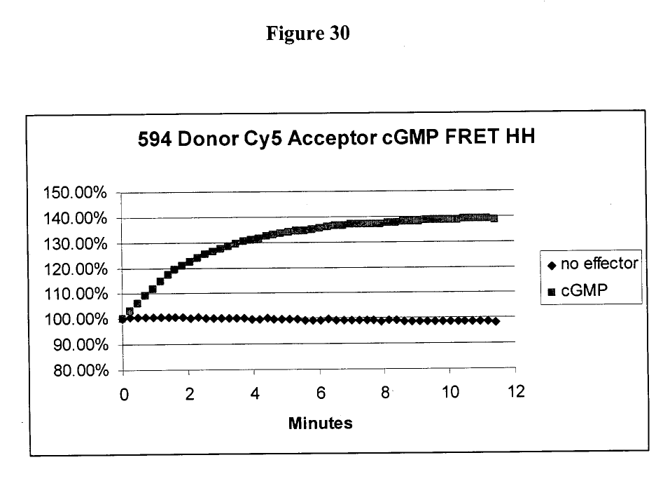

To investigate the use of other types of labels, various near-IR dyes were attached to the 3′-end of the cGMP model sensor. Alexa 594, Alexa 633 and Alexa 647 were purchased in the required hydrazide chemistry from Molecular Probes. The corresponding 5′-modified DNA quencher/acceptor oligos were chemically synthesized using Cy 3.5, Cy 5 and Cy 5.5 phosphoramidites from Glen Research. For the FRET assays, the sensor RNAs were annealed to the appropriate DNA oligos to yield the dye combinations outlined in Table 9. Cleavage reactions were initiated through addition of 800 μM cGMP under the previously established standard buffer conditions. The fluorescence signals were monitored on both the donor and acceptor dye emission wavelengths. Of all the combinations tested, combination A gave the best FRET signal (FIG. 30).

TABLE 9
Dye combinations for FRET assays in the near IR range.

| | Hammerhead NASM RNA | | | | DNA oligo (automated | | | |
|---|---|---|---|---|---|---|---|---|
| | (in vitro transcription, oxidation, hydrazide) | | | | synthesis, phosphoramidite) | | | |
| | Donor | EX | EM | FRET | Acceptor | EX | EM | FRET |
| A. | Alexa 594 (=Cy3.5) | 588 | 613 | ↑ good | Cy5 | 646 | 662 | ↓ medium |
| B. | Alexa 594 (=Cy3.5) | 588 | 613 | ↑ low | Cy5.5 | 683 | 707 | — |
| C. | Alexa 633 | 624 | 643 | — | Cy5.5 | 683 | 707 | — |
| | Acceptor | | | | Donor | | | |
| D. | Alexa 647 (=Cy5) | 649 | 666 | — | Cy3.5 | 588 | 604 | — |

Due to the availability and cost of reagents, as well as convenience of synthesis, FAM and dabcyl were chosen for most FRET experiments. However, other dyes and quenchers are expected to work sufficiently well.

The specific criteria for success included a demonstration that NASM assay CVs could approach 5% in the best of conditions, such as in solution in an aqueous buffer, buffered cola, and buffered coffee matrices. Experiments performed for five days demonstrated that a consistent concentration/response relationship could be achieved in aqueous buffer. Across five days, the slope of the concentration response relationship was 0.00110+/−0.00003, with a CV of 3%. cGMP values could be predicted from an average of these standard curves with a CV of 6% and an accuracy of >96%. In cola matrix that was buffered using HEPES and Tris to pH 7.5 and contained 35 mM Mg⁺⁺, slope was 0.00114+/−0.00002, with a CV of 2% and the predicted values had a CV of about 10% with an accuracy of >96%. The slope of the concentration/response relationship was not significantly altered by the cola matrix.

The intensity of the FRET signal generated by a sensor depends on a variety of factors. Apart from the purity of the sensor and quencher preparations, the pH of the assay buffer has a substantial effect. With increasing pH the basal fluorescence of the quenched sensor increases, while maximal FRET signal remains largely unchanged. Accordingly, the relative signal strength decreases at higher pH values. Since the first order rate analysis is independent of absolute signal values, this does not necessarily affect the measurements. A similar progressive reduction in signal was observed upon addition of magnesium chloride.

### Example 5

### Characteristics of Individual Sensors in FRET Format

The caffeine clone STC.43.29.Dl 1 (S2.caf.D11) and the aspartame clones AF.35.147.E4 (S3.asp.E4) and AF.35.147.A2 (S3.asp.A2), were adapted to a solution-based FRET format. The characteristics of these three NASM systems are described below.

I. Synthesis and Use of FRET Hammerhead Caffeine NASMs

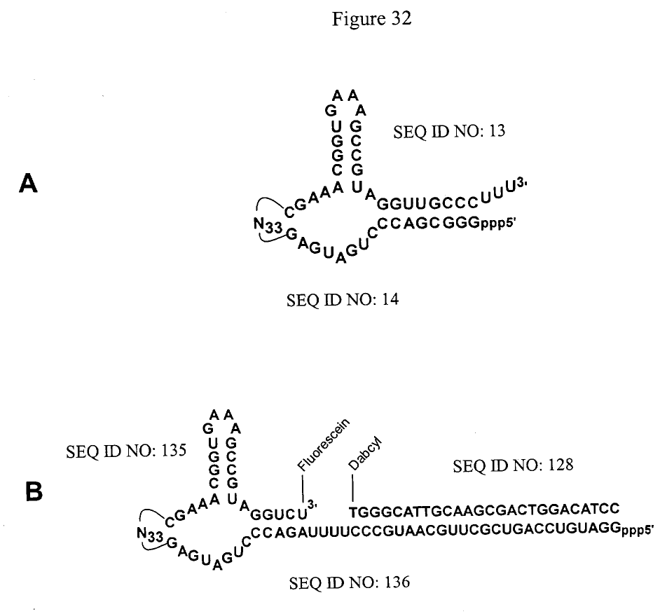

To synthesize the caffeine-dependent hammerhead RNA for FRET analysis, the structure of stem I was altered, and a fluorescent label was attached as shown in FIG. 32.

The DNA template for clone STC.43.29.D11 was amplified in a PCR reaction under standard conditions (20 mM Tris pH 8.4, 50 mM KCl, 2 mM MgCl₂, 0.5 mM each dNTP, 0.05 units/mL taq polymerase; cycle: 94° C. 1 min, 55° C. 1 min, 72° C. 1 min) that contained 5′-TAATACGACTCACTATAGGATGTCCAGTCGCTTGCAATGCCCTT TTAGACCCTGATGAG-3′ (SEQ ID NO: 131) and 5′-AGACCTACGGCTTTCACC GTTTCG-3′ (SEQ ID NO: 132) as primers. Subsequently, the amplification product was used as a template for in vitro transcription using the Ampliscribe T7 polymerase kit (Epicentre, Madison, Wis.) to yield RNA with the sequence 5′-GGAUGUCCAGUCGCUUGCAAUGCCCUUUUAGACCCUGAUGAGGAUCAUC GGACUUUGUCCUGUGGAGUAAGAUCGCGAAACGGUGAAAGCCGUAGGUCU-3′ (SEQ ID NO:133). The RNA (30 nmole in 300 μL H₂O) was then mixed with 150 μL 1.2 M sodium acetate pH 5.4 plus 150 μL of fresh 40 mM sodium metaperiodate in H₂O and incubated for 1 hour on ice in the dark. Following precipitation with 600 μL isopropanol, the 3′-oxidized RNA was resuspended in 450 μL H₂O and 150 μL 1.2M sodium acetate buffer pH 5.4 and 100 μL freshly made 40 mM fluorescein thiosemicarbazide in DMSO was added. The mixture was reacted for 2 hours at room temperature, after which the labeled nucleic acid was precipitated, purified by gel-electrophoresis on a 6% denaturing polyacrylamide gel, and resuspended in H₂O to a final concentration of 12 μM.

To perform fluorescence resonance energy transfer (FRET) measurements, fluorescein-labeled RNA (0.5 μL/assay point) and quencher oligo 5′-Dabcyl-TGGGCATTGCAAGCGACTGGACATCC-3′ (SEQ ID NO: 134) (30 pmole in 0.5 μL/assay point) in a total of 10 μL of 10 mM Tris pH 7.4, 50 mM NaCl were heated to 80° C. for 2 min. The mixture was then allowed to cool to room temperature, 20 μL H₂O and 20 μL 2×assay buffer (180 mM Hepes pH 7.0-8.0, 300 mM NaCl, 20 or 50 mM MgCl₂, 2 mM EDTA, 20 mM sodium phosphate) was added and the mixture was equilibrated for another 15 min. Cleavage reactions were performed in black 96-well microplates, and were started by mixing 35 μL of the above nucleic acid sensor solution with 35 μL of caffeine (20 μM-10 mM final concentration) in assay buffer (90 mM Hepes pH 7.0-8.0, 150 mM NaCl, 10 or 25 mM MgCl₂, 1 mM EDTA, 10 mM sodium phosphate). The fluorescence signals were monitored in a Fusion™ α-FP plate reader and the obtained rfu values were plotted against time. The apparent reaction rates were calculated assuming the 1^{st} order kinetic model equation: y=A(1−e^{kt})+NS (A: signal amplitude; k: observed catalytic rate; NS: nonspecific background signal) using a curve fit algorithm (KaleidaGraph, Synergy Software, Reading, Pa.). Dose-response curves were generated by plotting the calculated rates vs. the corresponding caffeine concentrations.

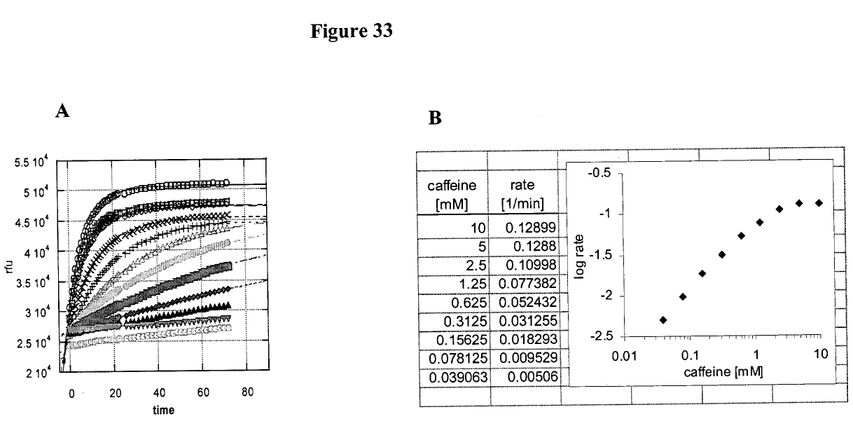

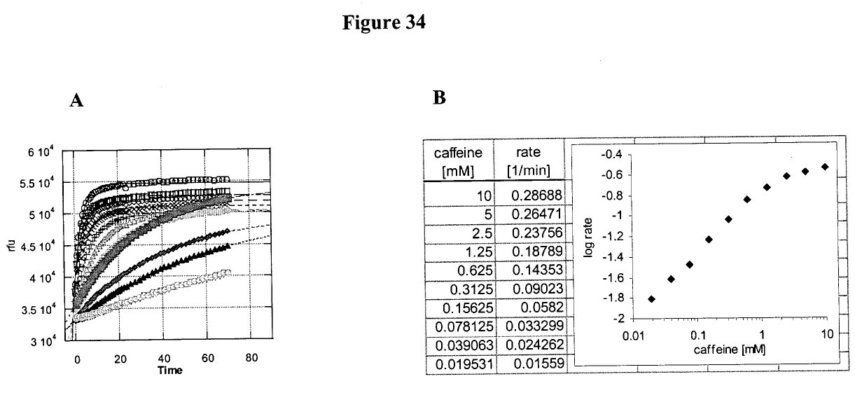

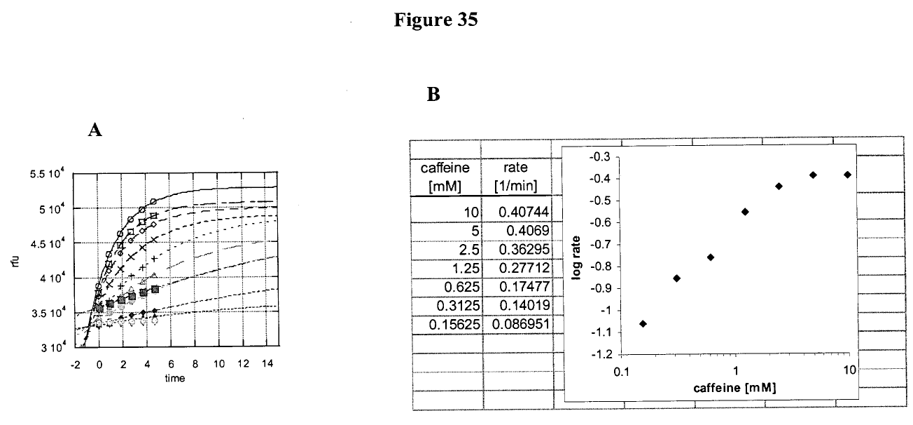

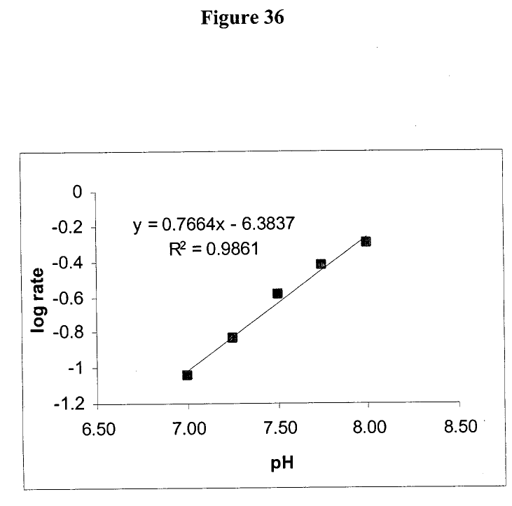

Measurements for clone STC.43.29.D11 were initially done at pH 7.0 with 10 mM (FIG. 33) or 25 mM MgCl₂ (FIG. 34), with caffeine concentrations ranging from 20 μM to 10 mM. The FRET signals developed rapidly, and showed sufficient discrimination within the first 5 minutes (FIG. 35). The rates were clearly dose-dependent between 1 and 5 mM caffeine, with a linear range between 20 μM and 1 mM, the apparent K_{d} of the sensor-caffeine interaction. In addition to the strong dependence on Mg²⁺, the pH had a significant effect on the reaction rates (FIG. 36).

II. Synthesis and Use of FRET Hammerhead Aspartame NASMs

The synthesis of aspartame-dependent FRET hammerhead sensor molecules was done similar as described for the caffeine NASM. The DNA templates for clones AF.35.147.A2 and AF35.147.E4 were amplified in a PCR reaction under standard conditions (20 mM Tris pH 8.4, 50 mM KCl, 2 mM MgCl₂, ˜0.5 mM each dNTP, 0.05 units/μL taq polymerase; cycle: 94° C. 1 min, 55° C. 1 min, 72° C. 1 min) that contained 5′-TAATACGACTCACTATAGGATGTCCAGTCGCTTGCAATGCCCTTTTAGACCC TGATGAG-3′ (SEQ ID NO: 131) and 5′-AGACCTACGGCTTTCACCGTTTCG-3′ (SEQ ID NO: 132) as primers. Subsequently, the amplification products were used as templates for in vitro transcriptions using Ampliscribe T7 polymerase kits (Epicentre, Madison, Wis.) to yield RNAs with the sequences

| | |
|---|---|
| 5′-GGAUGUCCAGUCGCUUGCAAUGCCCUUUUAGACCCUGAUGAGCAGGCAA | (A2, SEQ ID NO:137) |
| ACGUGCGCCUAGAAUGCAGACACCAACGAAACGGUGAAAGCCGUAGGUCU-3′ and | |
| 5′-GGAUGUCCAGUCGCUUGCAAUGCCCUUUUAGACCCUGAUGAGCGGUGCU | (E4, SEQ ID NO:138) |
| AGUUAGUUGCAGUUUCGGUUGUUACGCGAAACGGUGAAAGCCGUAGGUCU-3′. | |

The RNAs (30 nmole in 300 μL H₂O) were then mixed with 150 μL 1.2 M sodium acetate buffer pH 5.4 plus 150 μL of fresh 40 mM sodium metaperiodate in H₂O and incubated for 1 hour on ice in the dark. Following precipitation with 600 μL isopropanol, the 3′-oxidized RNAs were resuspended in 450 μL H₂O and 150 μL 1.2 M sodium acetate buffer pH 5.4 and 100 μL freshly made 40 mM fluorescein thiosemicarbazide in DMSO was added. The mixtures were reacted for 2 hours at room temperature, after which the labeled nucleic acids were precipitated, purified by gel-electrophoresis on a 6% denaturing polyacrylamide gel, and resuspended in H₂O to final concentrations of 12 μM.

To perform fluorescence resonance energy transfer measurements, fluorescein-labeled RNA (0.5 μL/assay point) and quencher oligo 5′-Dabcyl-TGGGCATTGCAAGCGACTGGACATCC-3′ (SEQ ID NO: 124) (30 μmole in 0.5 μL) in a total of 10 μL of 10 mM Tris pH 7.4, 50 mM NaCl were heated to 80° C. for 2 min. The mixture. was then allowed to cool to room temperature, 20 μL H₂O and 20 μL 2× assay buffer without Mg²⁺ (180 mM Hepes pH 7.0-8.0, 300 mM NaCl, 2 mM EDTA, 20 mM Sodium phosphate) were added and the mixture was equilibrated for another 15 min. Cleavage reactions were performed in black 96-well microplates, and were started by mixing 35 μL of the nucleic acid sensor solution with 35 μL of aspartame (20 μM-10 mM final concentration) in assay buffer containing 2×Mg²+(90 mM Hepes pH 7.0-8.0, 150 mM NaCl, 20-80 mM MgCl₂, 1 mM EDTA, 10 mM Sodium phosphate). The fluorescence signals were monitored in a Fusion™ α-FP plate reader and the obtained rfu values were plotted against time. The apparent reaction rates were calculated assuming the 1^{st} order kinetic model equation y=A(1−e^{-kt})+NS (A: signal amplitude; k: observed catalytic rate; NS: nonspecific background signal) using a curve fit algorithm (KaleidaGraph, Synergy Software, Reading, Pa.). Dose-response curves were generated by plotting the calculated rates vs. the corresponding aspartame concentrations.

A. Aspartame-S3.asp. E4

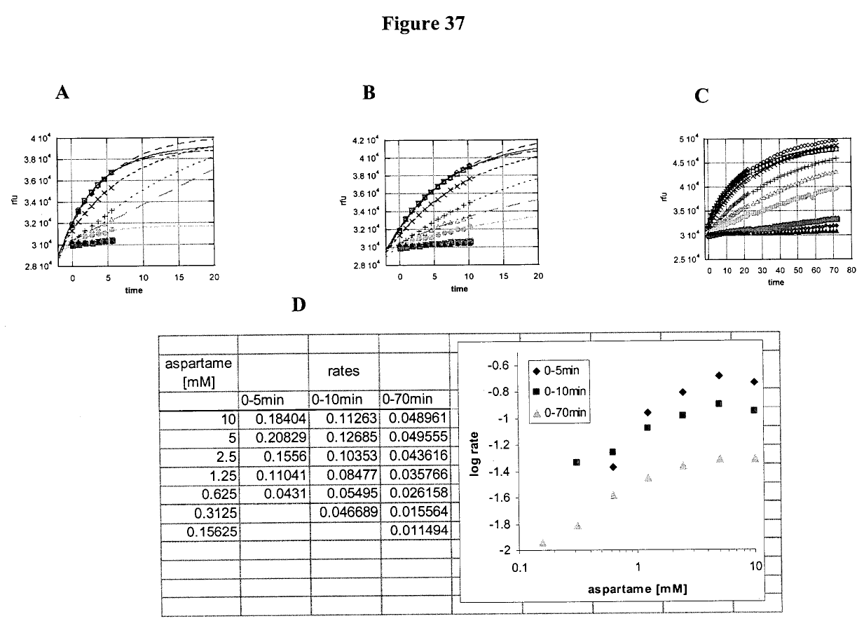

Measurements for clone AF.35.147.A2 were done at pH 7.0 with 10 mM MgCl₂ (FIG. 37), and aspartame concentrations ranging from 20 μM to 10 mM, with read times of 5, 10, and 70 minutes. For measurements that are recorded over a 70 min time, the rate-analysis showed a clear dose-dependence between 150 μM and 5 mM target. A shortening of the measurement time lead to loss in sensitivity and a reduction in detection range (FIG. 37). Additional experiments at pH 7.0-pH 8.0 show no significant pH-dependence of the reaction rate. Presumably, clone AF.35.147.A2 specifically recognizes the protonated α-amino group on aspartame. Shifting the pH to higher values may accelerate the ribozyme cleavage according to a general base mechanism, but may also reduce the concentration of the active form of target. These two effects might then cancel each other out.

In the hammerhead ribozyme sequence context used for selection, S3.asp.A2 is activated approximately 50-fold by 5 mM aspartame in a 20 minute assay, as analyzed by denaturing gel electrophoresis. In the solution-based FRET assay at pH 7.0 and 10 mM MgCl₂ cleavage is strongly target-dependent. Depending on the read-times used for analysis, significantly different catalytic rates (FIG. 37) are obtained. The reasons for this are unclear; one assumption is that this particular NASM system does not follow first order cleavage kinetics which renders the curve-fitting equation inappropriate.

The addition of 25 mM Mg²⁺ did not lead to a significant rate increase, but rather to a reduction of FRET signal. Under these conditions, rates were difficult to analyze and did not show any suitable dose-dependence

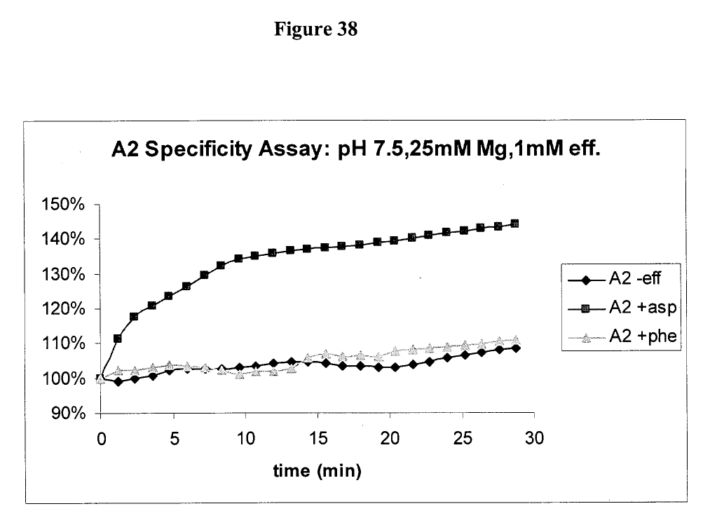

Phenylalanine activation of S3.asp.A2 was analyzed, as phenylalanine is listed as a component of cola. As shown in FIG. 38, S3.asp.A2 is not activated by 1 mM phenylalanine at pH 7.5, 25 mM MgCl₂.

B. Aspartame —S3.asp. E4

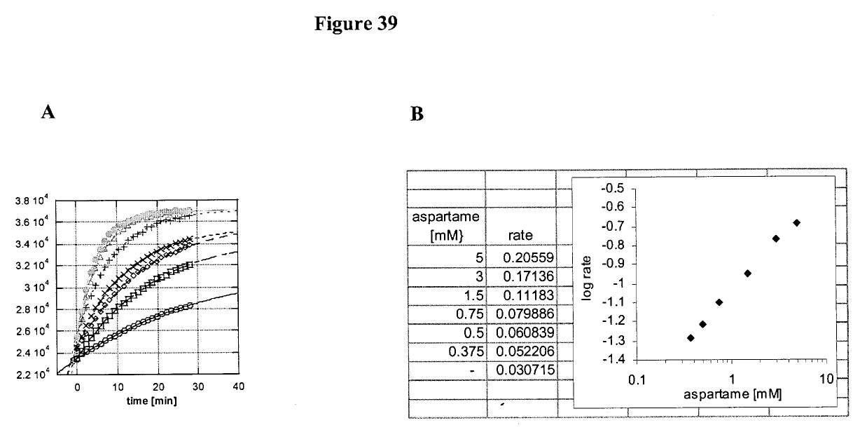

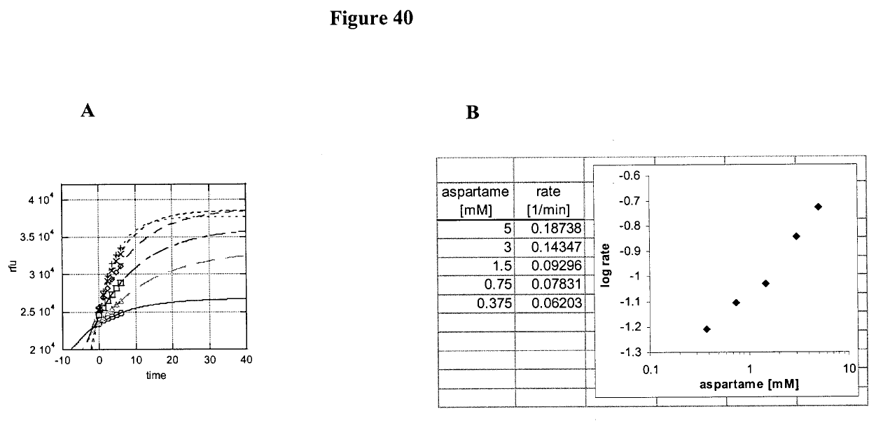

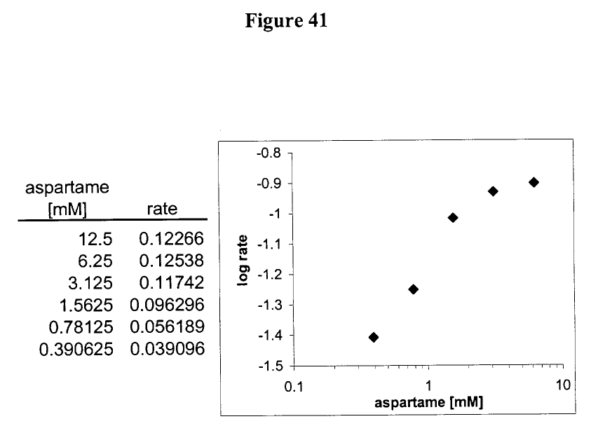

S3.asp.E4 yielded a clear dose-response curve at pH 7.5 and 40 mM MgCl₂, as shown in FIGS. 39 and 40, when data from either 30 (FIG. 39) or 5 minutes, 45 seconds (FIG. 40) were used to calculate rates (use of a five minute, 45 seconds range allowed inclusion of an additional data point). The catalytic rate for the background was ˜0.03 min⁻¹, which is distinctively below the rates for the aspartame-dependent reaction for the target concentrations measured. Additional measurements were done at 25 mM MgCl₂/pH 7.5 and also show a clear dose-response in the range of ˜400 μM-6.25 mM aspartame (FIG. 41). The cleavage rate showed a strong dependence on the Mg²⁺-concentration (Table 10, compare FIGS. 39 and 41). Similar to S2.caf.D11, this may ultimately be used to adjust the reaction rates to accommodate for shorter detection times and even lower target concentrations.

TABLE 10
pH - and Mg2⁺-dependence of catalytic rates for
clone E4 +/− 3 mM aspartame target.

| Buffer | rate (+target) | rate (−target) |
|---|---|---|
| pH 7.0, 25 mM MgCl2 | 0.1257 min⁻¹ | — |
| pH 7.5, 10 mM MgCl2 | 0.0086 min⁻¹ | — |
| pH 7.5, 25 mM MgCl2 | 0.1663 min⁻¹ | 0.0140 min⁻¹ |
| pH 7.5, 40 mM MgCl2 | 0.1714 min⁻¹ | 0.0307 min⁻¹ |
| pH 8.0, 25 mM MgCl2 | 0.2191 min⁻¹ | n/d (but visible) |

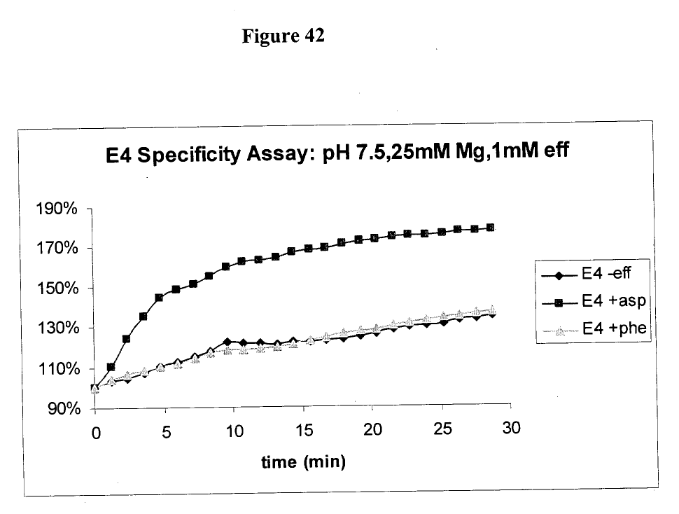

In addition, S3.asp.E4 also showed no cross reactivity with 1 mM phenylalanine as shown in FIG. 42, depicting a FRET assay using S3.asp.E4 in assay buffer pH 7.5, 25 mM MgCl₂ containing either 1 mM aspartame, phenylalanine, or no target. Since the reaction rate in the presence of 1 mM phenylalanine is essentially equivalent to the background rate, activation by phenylalanine under the 40 mM MgCl₂ assay conditions was not expected.

The pH and MgCl₂ dependence of this NASM system may allow it to function suitably under a variety of conditions.

### Example 6

### Solid Phase Assays

A. SPReeta

One method for attaching NASMs to a surface involves passive adsorption of neutravidin to the gold Surface Plasmon Resonance (SPR) sensor surface (via cysteine residues), prior to subsequent attachment of a biotinylated hammerhead NASM. Purified neutravidin (Pierce) was flowed over the SPReeta sensor surface at a concentration of 50-100 μg/mL. The successful adsorption of the neutravidin was monitored in real time using the protocol outlined below.

In the first step, the surface of a chip is cleaned by flowing a phosphate buffer solution (PBS) over the chip surface for two minutes at a flow rate of 0.3 mL/min, and subsequently flowing a solution having 120 mM NaOH and 1% Triton X100 over the chip surface for another two minutes at a flow rate of 0.3 mL/min. In the second step, neutravidin was bound to the surface of the chip by flowing a phosphate buffer solution (PBS) over the chip surface for two minutes at a flow rate of 0.3 mL/min, and subsequently flowing a solution containing 40 μg/mL in PBS (Pierce Catalog No. 31000) for ten minutes at a flow rate of 0.3 mL/min. Unbound neutravidin was washed off the surface of the chip by flowing a phosphate buffer solution (PBS) over the chip surface for two minutes at a flow rate of 0.3 mL/min, flowing a solution having 10 mM NaOH for another two minutes at a flow rate of 0.3 mL/min, and finally flowing a phosphate buffer solution (PBS) over the chip surface for two minutes at a flow rate of 0.3 mL/min. Typically, neutravidin binding to the chip surface results in approximately 2000 Refractive Index Units (RIUs).

One or more biotin-labeled macromolecules were attached to the surface of the chip by flowing a solution having the biotinylated molecule in PBS over the surface of the chip for ten minutes at a flow rate of 0.3 mL/min. Unbound biotinylated molecules were washed off the chip surface by flowing a phosphate buffer solution (PBS) over the chip surface for two minutes at a flow rate of 0.3 mL/min, flowing a solution having 10 mM NaOH for another two minutes at a flow rate of 0.3 mL/min, and finally flowing a phosphate buffer solution (PBS) over the chip surface for two minutes at a flow rate of 0.3 mL/min. Typically, the change in RIUs after a biotin-labeled macromolecule has been attached to the surface of the chip is around 400 RIUs for a 40 kD molecule.

To this end, a biotinylated cGMP hammerhead construct was prepared. This construct allowed for a maximal cleavage fragment (˜65 nt=21.5 kD), and thus maximal dynamic range in the observed SPR signal, with only 5 nt retained on the surface.

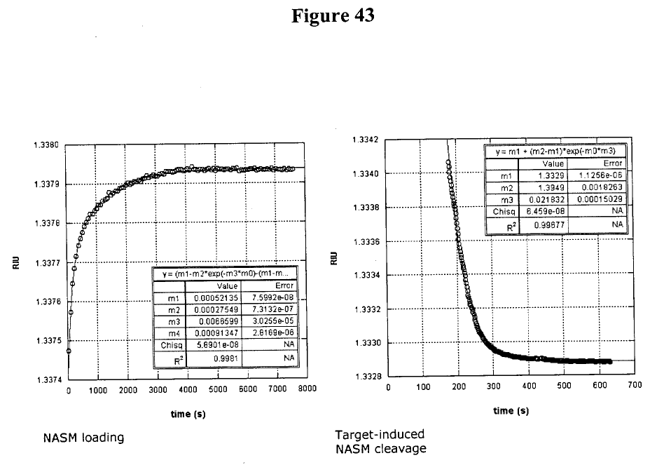

After characterizing the SPR signature and kinetic parameters for neutravidin adsorption, the surface immobilization of 3′-biotinylated cGMP NASM molecules was demonstrated. Initial experiments showed a significant reduction in Refractive Index Units (RIUs) upon introduction of excess (1 mM) target solution into the flow cell. For a 65 nt mass change, the maximum expected signal change (i.e., for complete cleavage of all surface-immobilized sensors) was ˜2145 RIU; the observed signal change was ˜1487 RIU, with a signal to noise ration (SNR)>100. Subsequent negative control experiments (using cAMP) confirmed the target-dependent origin of the observed signal. The cleavage/dissociation time course was fitted to a pseudo-first order rate function (correlation coefficient=0.9977), with an observed rate constant of ˜1 min⁻¹. The results of the SPReeta assay are shown in FIG. 43.

Initial cGMP dose-response data was acquired at concentrations of cGMP from 0 to 1 mM in hammerhead buffer with 20 mM MgCl₂. This data was generated using a single surface-adsorption step with neutravidin, followed by sequential addition of increasing concentrations of cGMP, with buffer flushes in between each cGMP addition. The SNR appeared to degrade at higher (>500 μM) cGMP concentrations (and thus extended times, for serial titrations), indicating that subsequent concentration-dependence measurements should be conducted as single-shot acquisitions consisting of a repeated cycle consisting of the following steps: surface stripping; cleaning in buffer; neutravidination; immobilization of cGMP; addition of cGMP.

B. Solid Phase FRET

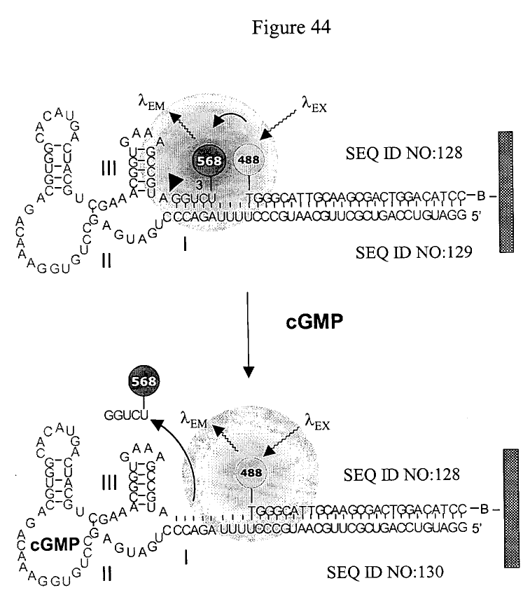

The performance of a modified form of the solution-phase FRET NASM system in a surface-immobilized configuration was investigated. The modified cGMP NASM system (with the quencher oligo replaced by a FAM (donor)-labeled and biotin-terminated oligo, and the FAM donor fluorophore on the ribozyme body replaced with an Alexafluor 568 acceptor fluorophore) was shown to recapitulate the catalytic rate and dynamic range measured previously with the FAM-quencher FRET system. The pre-hybridized ribozyme-oligo complex was immobilized in the neutravidin-coated wells of a 96 well plate (Pierce), as shown in FIG. 44.

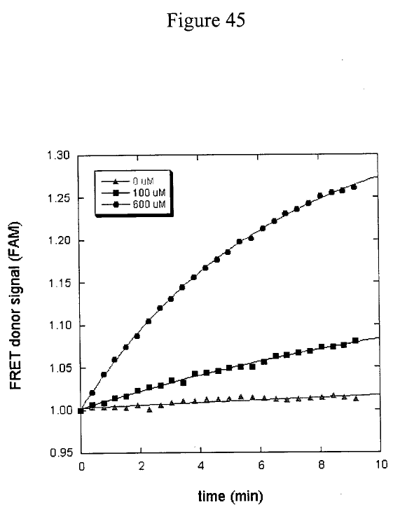

Data indicating FRET donor signal of the immobilized, modified cGMP NASM reaction with cGMP is shown in FIG. 45.

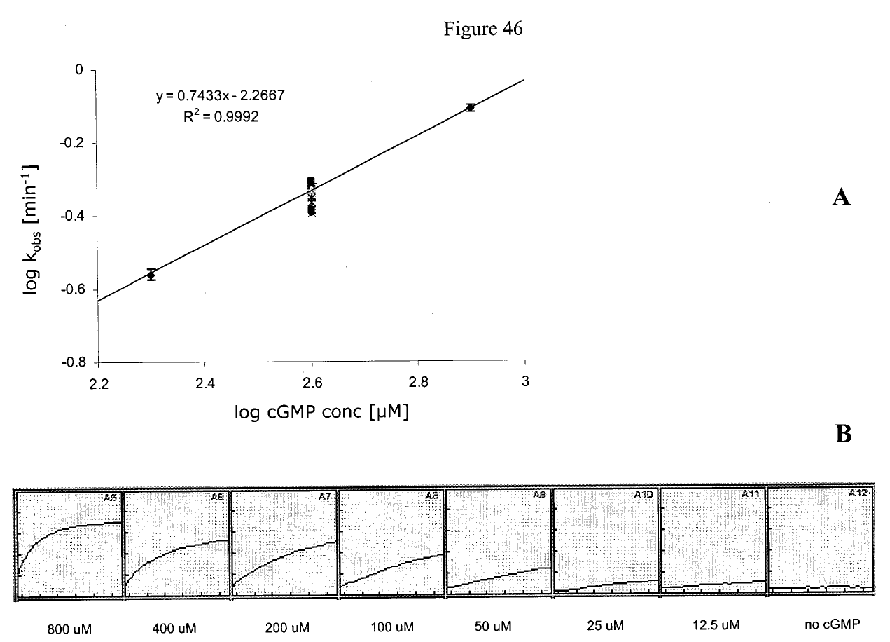

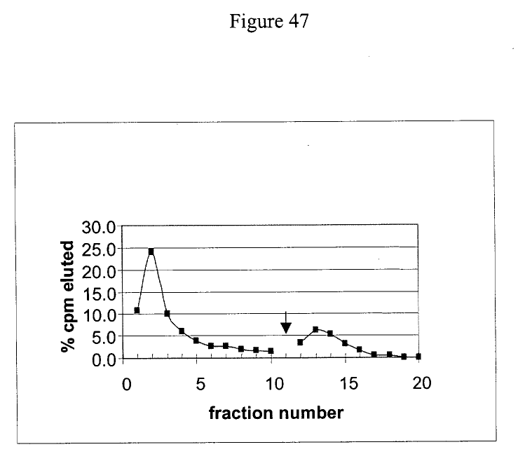

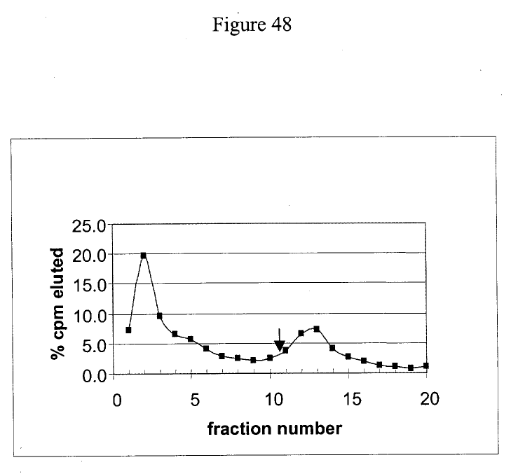

The concentration-dependence of the solid-phase NASM system to cGMP was measured, as shown in FIG. 46.

The fitted slopes of the catalytic rate vs. target concentration plots (i.e., dk_{obs}/d[cGMP], in min⁻¹ μM⁻¹) were compared for the solution and solid-phase systems. From this analysis, the catalytic rate for the solid-phase system was observed to have a roughly 9-fold lower dependence on target concentration than the solution-phase system. This result may be understood in terms of the effect of surface immobilization in reducing the effective availability of NASM molecules for interactions with target molecules, relative to the free solution-phase case.

The same cGMP sensors were also immobilized in an array spotted onto a streptavidin-impregnated membrane. After exposing the array to varying concentrations of target, the expected gain/loss in the donor/acceptor FRET signals were observed.

### Example 7

### Functional Assays

The HH33AG aspartame binding pool #1 was bound to the aspartame resin and eluted with buffered diet Pepsi in the elution washes, as shown in FIG. 47.

The HH33AG caffeine binding pool #1 was bound to the caffeine resin and eluted with buffered Pepsi in the elution washes, as shown in FIG. 48.

Variations, modifications, and other implementations of what is described herein will occur to those of ordinary skill in the art without departing from the spirit and scope as claimed. Accordingly, the invention is to be defined not by the preceding illustrative description but instead by the spirit and scope of the following claims.

We claim:

1. A nucleic acid sensor molecule comprising:
(a) a target modulation domain, wherein said target modulation domain recognizes caffeine;
(b) a linker domain; and
(c) a catalytic domain.

2. A nucleic acid sensor molecule comprising:
(a) a target modulation domain, wherein said target modulation domain recognizes aspartame;
(b) a linker domain; and
(c) a catalytic domain.

3. The nucleic acid sensor molecule of claim 1 or 2, wherein the catalytic domain comprises an optical signal generating unit.

4. The nucleic acid sensor molecule of claim 3, wherein said optical signal generating unit comprises at least one optical signaling moiety.

5. The nucleic acid sensor molecule of claim 3, wherein said optical signal generating unit comprises at least a first optical signaling moiety and a second optical signaling moiety.

6. The nucleic acid sensor molecule of claim 5, wherein said first and second signaling moieties change proximity to each other upon recognition of a target by the target modulation domain.

7. The nucleic acid sensor molecule of claim 5, wherein said first and second signaling moieties comprise a fluorescent donor and a fluorescent quencher, and recognition of a target by the target modulation domain results in an increase in detectable fluorescence of said fluorescent donor.

8. The nucleic acid sensor molecule of claim 6, wherein said first signaling moiety and said second signaling moiety comprise fluorescent energy transfer (FRET) donor and acceptor groups, and recognition of a target by the target modulation domain results in a change in distance between said donor and acceptor groups, thereby changing optical properties of said molecule.

9. The nucleic acid sensor molecule of claim 4, wherein said optical signaling moiety changes conformation upon recognition of a target by the target modulation domain, thereby resulting in a detectable optical signal.

10. The nucleic acid sensor molecule of claim 1 or 2, further comprising a detectable label.

11. The nucleic acid sensor molecule of claim 10 wherein the detectable label comprises at least one radioactive moiety.

12. The nucleic acid sensor of claim 10, wherein the detectable label comprises a fluorescent label.

13. The nucleic acid sensor of claim 12, wherein said fluorescent label is fluorescein, DABCYL, or a green fluorescent protein (GFP) moiety.

14. The nucleic acid sensor of claim 1 or 2, wherein said nucleic acid sensor further comprises an affinity capture tag label.

15. The nucleic acid sensor molecule of claim 1 or 2, wherein said nucleic acid sensor molecule includes at least one modified nucleotide.

16. The nucleic acid sensor molecule of claim 1 or 2, wherein said catalytic domain comprises an endonucleolytic ribozyme.

17. The nucleic acid sensor molecule of claim 16, wherein said endonucleolytic ribozyme is a hammerhead ribozyme.

18. The nucleic acid sensor molecule of claim 1 or 2, wherein said nucleic acid sensor molecule comprises RNA, DNA, or both RNA and DNA.

19. The nucleic acid sensor molecule of claim 1, wherein the nucleic acid sensor molecule is as shown in any one of SEQ ID NOs:21-78 or SEQ ID NOs:80-93.

20. The nucleic acid sensor molecule of claim 2, wherein the nucleic acid sensor molecule is as shown in any one of SEQ ID NOs:94-127.

21. A composition comprising the nucleic acid sensor molecule of any one of claims 1-20 and a buffer.

22. The composition of claim 21, further comprising an RNase inhibitor.

23. The composition of claim 22, wherein said RNase inhibitor is selected from the group consisting of Va-riboside, vanadyl, tRNA, polyu, RNaseln and RNaseOut.

24. The composition of claim 22 or 23, wherein said composition is substantially RNase-free.

25. A composition comprising at least one nucleic acid sensor molecule according to any one of claims 1-20, affixed to a substrate.

26. The composition of claim 25, wherein said substrate is glass, gold or other metal, silicon or other semiconductor material, nitrocellulose, nylon, or plastic.

27. The composition of claim 25, wherein the nucleic acid sensor molecule is covalently attached to said substrate.

28. The composition of claim 25, wherein the nucleic acid sensor molecule is non-covalently attached to said substrate.

29. The composition of claim 25, wherein the nucleic acid sensor molecule is immobilized to the substrate via hybridization of a terminal portion of the nucleic acid sensor molecule to an oligonucleotide that is bound to the surface of the substrate.

30. The composition of claim 25, wherein said composition comprises a plurality of nucleic acid sensor molecules immobilized to the substrate via hybridization of a terminal portion of the nucleic acid sensor molecule to an array of oligonucleotides bound to the substrate at spatially discrete regions.

31. The substrate of claim 25, wherein said substrate comprises at least 50 nucleic acid sensor molecules.

32. The substrate of claim 25, wherein said substrate comprises at least 250 nucleic acid sensor molecules.

33. A system for detecting caffeine, comprising a composition comprising a target modulation domain which recognizes caffeine, according to any one of claims 25-32 and a detector in communication with said composition, wherein said detector is capable of detecting a signal generated upon recognition of a target molecule by a nucleic acid sensor molecule.

34. A system for detecting aspartame, comprising a composition comprising a target modulation domain which recognizes aspartame, according to any one of claims 25-32 and a detector in communication with said composition, wherein said detector is capable of detecting a signal generated upon recognition of a target molecule by a nucleic acid sensor molecule.

35. The system of claim 33 or 34, further comprising a light source in optical communication with said composition.

36. The system of claim 33 or 34, further comprising a processor for processing optical signals detected by the detector.

37. A method of identifying or detecting caffeine in a sample, the method comprising:
contacting a sample suspected of containing caffeine with a nucleic acid molecule which recognizes caffeine, according to any one of claims 3-19 or 21-32, wherein a change in the signal generated by the optical signal generating unit or detectable label indicates the presence of caffeine in said sample.

38. The method of claim 37 further comprising quantifying the change in signal generated by the optical signal generating unit or detectable label to quantify the amount of caffeine in the sample.

39. The method of claim 37 or 38 wherein the sample is a process solution.

40. The method of claim 37 or 38 wherein the sample is coffee or a soft drink.

41. A method of identifying or detecting aspartame in a sample, the method comprising:
contacting a sample suspected of containing aspartame with a nucleic acid molecule which recognizes aspartame, according to any one of claims 3-18 or 20-32, wherein a change in the signal generated by the optical signal generating unit or detectable label indicates the presence of aspartame in said sample.

42. The method of claim 41 further comprising quantifying the change in signal generated by the optical signal generating unit or detectable label to quantify the amount of aspartame in the sample.

43. The method of claim 41 or 42 wherein the sample is a process solution.

44. The method of claim 41 or 42 wherein the sample is coffee or a soft drink.

45. A diagnostic system for identifying or detecting caffeine, the diagnostic system comprising a nucleic acid sensor molecule according to any one of claims 3-19 or 21-32 and
a detector in communication with said nucleic acid sensor molecule, wherein said detector detects changes in the signal generated by the optical signal generating unit or detectable label of said nucleic acid sensor.

46. The diagnostic system of claim 45, further comprising a processor for processing signals detected by the detector.

47. A diagnostic system for identifying or detecting aspartame, the diagnostic system comprising
a nucleic acid sensor molecule according to any one of claims 3-18 or 20-32 and a detector in communication with said nucleic acid sensor molecule, wherein said detector detects changes in the signal generated by the optical signal generating unit or detectable label of said nucleic acid sensor.

48. The diagnostic system of claim 47, further comprising a processor for processing signals detected by the detector.
